(19) United States
(12) Patent Application Publication
(10) Pub. No.: US 2024/0199651 A1
(43) Pub. Date: Jun. 20, 2024

(54) ANTIBACTERIAL COMPOUNDS

(71) Applicant: JANSSEN SCIENCES IRELAND UNLIMITED COMPANY, Co. Cork (IE)

(72) Inventors: Jérôme Émile Georges GUILLEMONT, Andé (FR); Magali Madeleine Simone MOTTE, Louviers (FR); Dirk Antonie LAMPRECHT, Turnhout (BE); José Manuel BARTOLOMÉ-NEBREDA, Toledo (ES)

(21) Appl. No.: 18/550,342

(22) PCT Filed: Mar. 16, 2022
(86) PCT No.: PCT/EP2022/056772

§ 371 Date: Sep. 13, 2023

(30) Foreign Application Priority Data
Mar. 17, 2021 (EP) 21163040.5

(51) Int. Cl. C07D 519/00; A61K 45/06; A61P 31/06

(52) U.S. Cl. CPC C07D 519/00; A61K 45/06; A61P 31/06

(57) ABSTRACT

The present invention relates to the compounds (I) wherein the integers are as defined in the description, and where the compounds may be useful as medicaments, for instance for use in the treatment of tuberculosis.

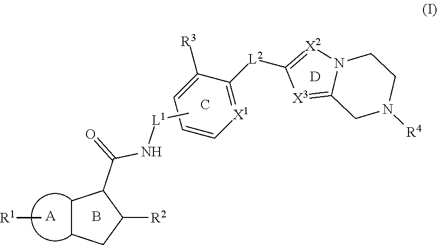

The present invention relates to novel compounds. The invention also relates to such compounds for use as a pharmaceutical and further for the use in the treatment of bacterial diseases, including diseases caused by pathogenic mycobacteria such as Mycobacterium tuberculosis. Such compounds may work by interfering with ATP synthase in M. tuberculosis, with the inhibition of cytochrome bc₁ activity as the primary mode of action. Hence, primarily, such compounds are antitubercular agents.

### BACKGROUND OF THE INVENTION

Mycobacterium tuberculosis is the causative agent of tuberculosis (TB), a serious and potentially fatal infection with a world-wide distribution. Estimates from the World Health Organization indicate that more than 8 million people contract TB each year, and 2 million people die from tuberculosis yearly. In the last decade, TB cases have grown 20% worldwide with the highest burden in the most impoverished communities. If these trends continue, TB incidence will increase by 41% in the next twenty years. Fifty years since the introduction of an effective chemotherapy, TB remains after AIDS, the leading infectious cause of adult mortality in the world. Complicating the TB epidemic is the rising tide of multi-drug-resistant strains, and the deadly symbiosis with HIV. People who are HIV-positive and infected with TB are 30 times more likely to develop active TB than people who are HIV-negative and TB is responsible for the death of one out of every three people with HIV/AIDS worldwide.

Existing approaches to treatment of tuberculosis all involve the combination of multiple agents. For example, the regimen recommended by the U.S. Public Health Service is a combination of isoniazid, rifampicin and pyrazinamide for two months, followed by isoniazid and rifampicin alone for a further four months. These drugs are continued for a further seven months in patients infected with HIV. For patients infected with multi-drug resistant strains of M. tuberculosis, agents such as ethambutol, streptomycin, kanamycin, amikacin, capreomycin, ethionamide, cycloserine, ciprofoxacin and ofloxacin are added to the combination therapies. There exists no single agent that is effective in the clinical treatment of tuberculosis, nor any combination of agents that offers the possibility of therapy of less than six months' duration.

There is a high medical need for new drugs that improve current treatment by enabling regimens that facilitate patient and provider compliance. Shorter regimens and those that require less supervision are the best way to achieve this. Most of the benefit from treatment comes in the first 2 months, during the intensive, or bactericidal, phase when four drugs are given together; the bacterial burden is greatly reduced, and patients become noninfectious. The 4- to 6-month continuation, or sterilizing, phase is required to eliminate persisting bacilli and to minimize the risk of relapse. A potent sterilizing drug that shortens treatment to 2 months or less would be extremely beneficial. Drugs that facilitate compliance by requiring less intensive supervision also are needed. Obviously, a compound that reduces both the total length of treatment and the frequency of drug administration would provide the greatest benefit.

Complicating the TB epidemic is the increasing incidence of multi-drug-resistant strains or MDR-TB. Up to four percent of all cases worldwide are considered MDR-TB—those resistant to the most effective drugs of the four-drug standard, isoniazid and rifampin. MDR-TB is lethal when untreated and cannot be adequately treated through the standard therapy, so treatment requires up to 2 years of “second-line” drugs. These drugs are often toxic, expensive and marginally effective. In the absence of an effective therapy, infectious MDR-TB patients continue to spread the disease, producing new infections with MDR-TB strains. There is a high medical need for a new drug with a new mechanism of action, which is likely to demonstrate activity against drug resistant, in particular MDR strains.

The term “drug resistant” as used hereinbefore or hereinafter is a term well understood by the person skilled in microbiology. A drug resistant Mycobacterium is a Mycobacterium which is no longer susceptible to at least one previously effective drug; which has developed the ability to withstand antibiotic attack by at least one previously effective drug. A drug resistant strain may relay that ability to withstand to its progeny. Said resistance may be due to random genetic mutations in the bacterial cell that alters its sensitivity to a single drug or to different drugs.

MDR tuberculosis is a specific form of drug resistant tuberculosis due to a bacterium resistant to at least isoniazid and rifampicin (with or without resistance to other drugs), which are at present the two most powerful anti-TB drugs. Thus, whenever used hereinbefore or hereinafter “drug resistant” includes multi drug resistant.

Another factor in the control of the TB epidemic is the problem of latent TB. In spite of decades of tuberculosis (TB) control programs, about 2 billion people are infected by M. tuberculosis, though asymptomatically. About 10% of these individuals are at risk of developing active TB during their lifespan. The global epidemic of TB is fueled by infection of HIV patients with TB and rise of multi-drug resistant TB strains (MDR-TB). The reactivation of latent TB is a high risk factor for disease development and accounts for 32% deaths in HIV infected individuals. To control TB epidemic, the need is to discover new drugs that can kill dormant or latent bacilli. The dormant TB can get reactivated to cause disease by several factors like suppression of host immunity by use of immunosuppressive agents like antibodies against tumor necrosis factor α or interferon-γ. In case of HIV positive patients the only prophylactic treatment available for latent TB is two-three months regimens of rifampicin, pyrazinamide. The efficacy of the treatment regime is still not clear and furthermore the length of the treatments is an important constrain in resource-limited environments. Hence there is a drastic need to identify new drugs, which can act as chemoprophylatic agents for individuals harboring latent TB bacilli.

The tubercle bacilli enter healthy individuals by inhalation; they are phagocytosed by the alveolar macrophages of the lungs. This leads to potent immune response and formation of granulomas, which consist of macrophages infected with M. tuberculosis surrounded by T cells. After a period of 6-8 weeks the host immune response cause death of infected cells by necrosis and accumulation of caseous material with certain extracellular bacilli, surrounded by macrophages, epitheloid cells and layers of lymphoid tissue at the periphery. In case of healthy individuals, most of the mycobacteria are killed in these environments but a small proportion of bacilli still survive and are thought to exist in a non-replicating, hypometabolic state and are tolerant to killing by anti-TB drugs like isoniazid. These bacilli can remain in the altered physiological environments even for individual's lifetime without showing any clinical symptoms of disease. However, in 10% of the cases these latent bacilli may reactivate to cause disease. One of the hypothesis about development of these persistent bacteria is patho-physiological environment in human lesions namely, reduced oxygen tension, nutrient limitation, and acidic pH. These factors have been postulated to render these bacteria phenotypically tolerant to major anti-mycobacterial drugs.

In addition to the management of the TB epidemic, there is the emerging problem of resistance to first-line antibiotic agents. Some important examples include penicillin-resistant Streptococcus pneumoniae, vancomycin-resistant enterococci, methicillin-resistant Staphylococcus aureus, multi-resistant salmonellae.

The consequences of resistance to antibiotic agents are severe. Infections caused by resistant microbes fail to respond to treatment, resulting in prolonged illness and greater risk of death. Treatment failures also lead to longer periods of infectivity, which increase the numbers of infected people moving in the community and thus exposing the general population to the risk of contracting a resistant strain infection.

Hospitals are a critical component of the antimicrobial resistance problem worldwide. The combination of highly susceptible patients, intensive and prolonged antimicrobial use, and cross-infection has resulted in infections with highly resistant bacterial pathogens.

Self-medication with antimicrobials is another major factor contributing to resistance. Self-medicated antimicrobials may be unnecessary, are often inadequately dosed, or may not contain adequate amounts of active drug.

Patient compliance with recommended treatment is another major problem. Patients forget to take medication, interrupt their treatment when they begin to feel better, or may be unable to afford a full course, thereby creating an ideal environment for microbes to adapt rather than be killed.

Because of the emerging resistance to multiple antibiotics, physicians are confronted with infections for which there is no effective therapy. The morbidity, mortality, and financial costs of such infections impose an increasing burden for health care systems worldwide.

Therefore, there is a high need for new compounds to treat bacterial infections, especially mycobacterial infections including drug resistant and latent mycobacterial infections, and also other bacterial infections especially those caused by resistant bacterial strains.

Anti-infective compounds for treating tuberculosis have been disclosed in e.g. international patent application WO 2011/113606. Such a document is concerned with compounds that would prevent M. tuberculosis multiplication inside the host macrophage and relates to compounds with a bicyclic core, imidazopyridines, which are linked (e.g. via an amido moiety) to e.g. an optionally substituted benzyl group.

International patent application WO 2014/015167 also discloses compounds that are disclosed as being of potential use in the treatment of tuberculosis. Such compounds disclosed herein have a bicycle (a 5,5-fused bicycle) as an essential element, which is substituted by a linker group (e.g. an amido group), which itself may be attached to another bicycle or aromatic group. Such compounds in this document do not contain a series of more than three rings.

Journal article Nature Medicine, 19, 1157-1160 (2013) by Pethe et al “Discovery of Q203, a potent clinical candidate for the treatment of tuberculosis” identifies a specific compound that was tested against M. tuberculosis. This compound Q203 is depicted below.

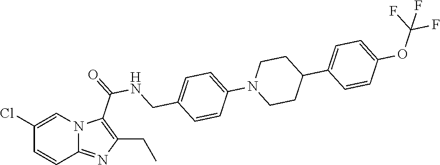 [SMILES: CCc1nc2ccc(Cl)cn2c1C(=O)NCc1ccc(N2CCC(c3ccc(OC(F)(F)F)cc3)CC2)cc1]

This clinical candidates is also discussed in journal article, J. Medicinal Chemistry, 2014, 57 (12), pp 5293-5305. It is stated to have activity against MDR tuberculosis, and have activity against the strain M. tuberculosis H37Rv at a MIC₅₀ of 0.28 nM inside macrophages. Positive control data (using known anti-TB compounds bedaquiline, isoniazid and moxifloxacin) are also reported. This document also suggests a mode of action, based on studies with mutants. It postulates that it acts by interfering with ATP synthase in M. tuberculosis, and that the inhibition of cytochrome bc₁ activity is the primary mode of action. Cytochrome bc₁ is an essential component of the electron transport chain required for ATP synthesis. It appeared that Q203 was highly active against both replicating and non-replicating bacteria.

International patent application WO 2015/014993 also discloses compounds as having activity against M. tuberculosis, as do international patent applications WO 2014/4015167, WO 2017/001660, WO 2017/001661, WO 2017/216281 and WO 2017/216283. International patent applications WO 2013/033070 and WO 2013/033167 disclose various compounds as kinase modulators.

The purpose of the present invention is to provide compounds for use in the treatment of bacterial diseases, particularly those diseases caused by pathogenic bacteria such as Mycobacterium tuberculosis (including the latent disease and including drug resistant M. tuberculosis strains). Such compounds may also be novel and may act by interfering with ATP synthase in M. tuberculosis, with the inhibition of cytochrome bc₁ activity being considered the primary mode of action.

### SUMMARY OF THE INVENTION

There is now provided a compound of formula (I)

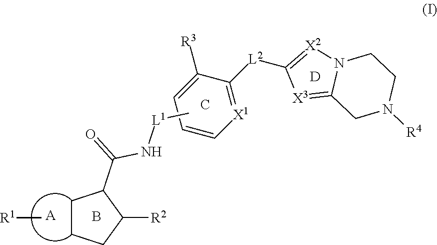

wherein
A is a 5- or 6-membered ring, which may be aromatic or non-aromatic, and optionally containing 1 or 2 heteroatoms selected from nitrogen, sulfur and oxygen;
B is a 5-membered aromatic ring containing 1 or 2 nitrogen heteroatoms;
X¹ represents ═N— or ═C(R^{10a})— (hence the C ring is phenyl or pyridyl);
one of X² and X³ (in the D ring) is ═N— and the other represents ═N— or ═C(R^{10b})—.
L¹ represents a linker group, and hence may be —C(R^{12a})(R^{12b})— or C₂₋₄ alkylene optionally substituted by one or more substituents selected from halo and —OC₁₋₃ alkyl;

L² represents an optional linker group, and hence may be a direct bond, —O—, —OCH₂—, —C(R^{12c})(R^{12d})— or C₂₋₄ alkylene optionally substituted by one or more substituents selected from halo and —OC₁₋₃ alkyl; or L² may represent a 4-, 5- or 6-membered aromatic or non-aromatic cyclic linker group, optionally containing one or two heteroatoms preferably selected from nitrogen, oxygen and sulfur, optionally substituted by one or more substitutents selected from halo and C₁₋₃ alkyl (itself optionally substituted by one or more fluoro atoms);
R¹ represents one or more (e.g. one, two or three) optional substituents independently selected from selected from halo (e.g. Cl, F), —R^{5a}, —O—R^{5b}, —C(═O)—R^{5c}, —C(═O)—N(R⁶)(R⁷), —CN and —N(R^{6a})R^{6b}; or any two R¹ groups may be taken together (when attached to adjacent atoms of the A ring) to form a 5- or 6-membered ring optionally containing one or two heteroatoms, and which ring is optionally substituted by one or two C₁-3 alkyl substituents;
R² is —C₁₋₄ alkyl optionally substituted by one or more substituents selected from halo and —OC₁₋₃ alkyl;
R³ represents a substituent selected from H, F, —C₁₋₃ alkyl and —O—C₁₋₃ alkyl;
R⁴ is H, —R^{8a}, —C(═O)—R^{8b}, —SO₂—R⁹ or Het¹;
R^{5a} and R^{5b} independently represent hydrogen or —C₁₋₄ alkyl (which, as mentioned herein) may be linear, branched or cyclic alkyl) optionally substituted by one or more substituents selected from halo (e.g. F), —O—CH₃ and phenyl;
R^{5c} is —C₁₋₃ alkyl;
R⁶ and R⁷ are independently selected from H and —C₁₋₃ alkyl;
R^{6a} and R^{6b} independently represent H, C₁₋₆ alkyl or R^{6a} and R^{6b} are linked together to form a 3- to 6-membered ring;
R^{8a} represents —C₁₋₄ alkyl, optionally substituted by one or more substituents selected from halo, —OC₁₋₃ alkyl, —CN and Het²;
R^{8b} is hydrogen or —C₁₋₃ alkyl (optionally substituted by one or more fluoro atoms);
R⁹ is Het³, —N(R^{6c})R^{6d} or —C₁₋₄ alkyl optionally substituted by one or more substituents selected from halo (e.g. F) and —O—CH₃;
R^{6c} and R^{6d} independently represent H, C₁₋₆ alkyl or R^{6c} and R^{6d} are linked together to form a 3- to 6-membered ring;
R^{10a} and R^{10b} independently represent H, halo, C₁₋₄ alkyl (itself optionally substituted by one or more, e.g. one, substituent(s) selected from fluoro, —CN, —R^{11a}, —OR^{11b}, —N(R^{11c})R^{11d} and/or —C(O)N(R^{11e})R^{11f}) or —O—C₁₋₄ alkyl (itself optionally substituted by one or more, e.g. one, substituent(s) selected from fluoro, —R¹¹, —OR^{11h} and/or —N(R¹¹ⁱ)R^{11j});
R^{11a}, R^{11b}, R^{11c}, R^{11d}, R^{11e}, R^{11f}, R^{11g}, R^{11h}, R¹¹ⁱ and R^{11j} independently represent hydrogen or C₁₋₃ alkyl (optionally substituted by one or more fluoro atoms);
R^{12a} and R^{12b} independently represent hydrogen or C₁₋₃ alkyl; or R^{12a} and R^{12b} are linked together to form a 3- to 6-membered ring;
R^{12c} and R^{12d} independently represent hydrogen or C₁₋₃ alkyl; or R^{12c} and R^{12d} are linked together to form a 3- to 6-membered ring;
   Het¹, Het² and Het³ independently represent a 5- or 6-membered aromatic ring containing one or two heteroatoms, preferably selected from nitrogen, oxygen and sulfur, optionally substituted by one or more substitutents selected from halo and C₁₋₃ alkyl (itself optionally substituted by one or more fluoro atoms),
   or a pharmaceutically-acceptable salt thereof,
   which compounds may be referred to herein as “compounds of the invention”.
There is also provided a compound of formula (I)

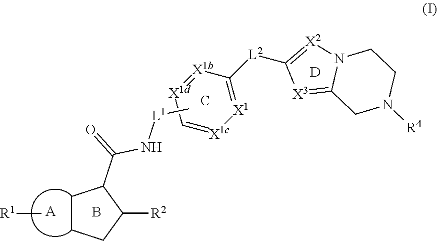

wherein
A is a 5- or 6-membered ring, which may be aromatic or non-aromatic, and optionally containing 1 or 2 heteroatoms selected from nitrogen, sulfur and oxygen;
B is a 5-membered aromatic ring containing 1 or 2 nitrogen heteroatoms;
X¹ represents ═N— or ═C(R^{10a})—.
X^{1b} represents ═N— or ═C(R³)—.
X^{1c} represents ═C(R^{10a}) or ═N—;
X^{1d} represents ═C(R^{10a}) or ═N—, and wherein a maximum of two of X¹, X^{1b}, X^{1c} and C^{1d} may represent ═N— (and hence the C ring may be phenyl, pyridyl, primidinyl);
one of X² and X³ (in the D ring) is ═N— and the other represents ═N— or ═C(R^{10b})—.
L¹ represents a linker group, and hence may be —C(R^{12a})(R^{12b})— or C₂₋₄ alkylene optionally substituted by one or more substituents selected from halo and —OC₁₋₃ alkyl;
L¹ may be situated para or meta relative to L² (and hence may be attached to either X^{1d} or the carbon atom inbetween X^{1d} and X^{1c});
L² represents an optional linker group, and hence may be a direct bond, —O—, —OCH₂—, —C(R^{12c})(R^{12d})— or C₂₋₄ alkylene optionally substituted by one or more substituents selected from halo and —OC₁₋₃ alkyl; or L² may represent a 4-, 5- or 6-membered aromatic or non-aromatic cyclic linker group, optionally containing one or two heteroatoms preferably selected from nitrogen, oxygen and sulfur, optionally substituted by one or more substitutents selected from halo and C₁₋₃ alkyl (itself optionally substituted by one or more fluoro atoms);
R¹ represents one or more (e.g. one, two or three) optional substituents independently selected from selected from halo (e.g. Cl, F), —R^{5a}, —O—R^{5b}, —C(═O)—R^{5c}, —C(═O)—N(R⁶)(R⁷), —CN and —N(R^{6a})R^{6b}; or any two R¹ groups may be taken together (when attached to adjacent atoms of the A ring) to form a 5- or 6-membered ring optionally containing one or two heteroatoms, and which ring is optionally substituted by one or two C₁₋₃ alkyl substituents;
R² is —C₁₋₄ alkyl (including C₃₋₄ cycloalkyl) optionally substituted by one or more substituents selected from halo and —OC₁₋₃ alkyl;
R³ represents a substituent selected from H, F, —C₁₋₃ alkyl and —O—C₁₋₃ alkyl;
R⁴ is H, —R^{8a}, —C(═O)—R^{8b}, —SO₂—R⁹ or Het¹;
R^{5a} and R^{5b} independently represent hydrogen or —C₁₋₄ alkyl (which, as mentioned herein) may be linear, branched or cyclic alkyl) optionally substituted by one or more substituents selected from halo (e.g. F), —O—CH₃ and phenyl;
R^{5c} is —C₁₋₃ alkyl;
R⁶ and R⁷ are independently selected from H and —C₁₋₃ alkyl;
R^{6a} and R^{6b} independently represent H, C₁₋₆ alkyl or R^{6a} and R^{6b} are linked together to form a 3- to 6-membered ring;
R^{8a} represents —C₁₋₄ alkyl, optionally substituted by one or more substituents selected from halo, —OC₁₋₃ alkyl, —CN and Het²;
R^{8b} is hydrogen or —C₁₋₃ alkyl (optionally substituted by one or more fluoro atoms);
R⁹ is Het³, —N(R^{6c})R^{6d} or —C₁₋₄ alkyl optionally substituted by one or more substituents selected from halo (e.g. F) and —O—CH₃;
R^{6c} and R^{6d} independently represent H, C₁₋₆ alkyl or R^{6c} and R^{6d} are linked together to form a 3- to 6-membered ring;
R^{10a} and R^{10b} independently represent H, halo, C₁₋₄ alkyl (itself optionally substituted by one or more, e.g. one, substituent(s) selected from fluoro, —CN, —R^{11a}, —OR^{11b}, —N(R^{11c})R^{11d} and/or —C(O)N(R^{11e})R^{11f}) or —O—C₁₋₄ alkyl (itself optionally substituted by one or more, e.g. one, substituent(s) selected from fluoro, —R^{11g}, —OR^{11h} and/or —N(R¹¹ⁱ)R^{11j});
R^{11a}, R^{11b}, R^{11c}, R^{11d}, R^{11e}, R^{11f}, R^{11g}, R^{11h}, R¹¹ⁱ and R^{11j} independently represent hydrogen or C₁₋₃ alkyl (optionally substituted by one or more fluoro atoms);
R^{12a} and R^{12b} independently represent hydrogen or C₁₋₃ alkyl; or R^{12a} and R^{12b} are linked together to form a 3- to 6-membered ring;
R^{12c} and R^{12d} independently represent hydrogen or C₁₋₃ alkyl; or R^{12c} and R^{12d} are linked together to form a 3- to 6-membered ring;

Het¹, Het² and Het³ independently represent a 5- or 6-membered aromatic ring containing one or two heteroatoms, preferably selected from nitrogen, oxygen and sulfur, optionally substituted by one or more substitutents selected from halo and C₁₋₃ alkyl (itself optionally substituted by one or more fluoro atoms),

or a pharmaceutically-acceptable salt thereof,
which compounds may also be referred to herein as “compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent.

Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group). In a similar way, C_{1-q} alkylene groups represent C_{1-q} alkyl linker groups, i.e. —CH₂— (C₁ alkylene or methylene), —CH₂CH₂—, etc according to the number “q” of carbon atoms.

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo.

Heterocyclic groups when referred to herein may include aromatic or non-aromatic heterocyclic groups, and hence encompass heterocycloalkyl and hetereoaryl. Equally, “aromatic or non-aromatic 5- or 6-membered rings” may be heterocyclic groups (as well as carbocyclic groups) that have 5- or 6-members in the ring.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like.

Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Heterocycloalkyl mentioned herein may be stated to be specifically monocyclic or bicyclic.

Aromatic groups may be aryl or heteroaryl. Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring.

However, when aryl groups are polycyclic (e.g. bicyclic or tricyclic), they are preferably linked to the rest of the molecule via an aromatic ring. Most preferred aryl groups that may be mentioned herein are “phenyl”.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring.

However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Heteroaryl groups mentioned herein may be stated to be specifically monocyclic or bicyclic. When heteroaryl groups are polycyclic in which there is a non-aromatic ring present, then that non-aromatic ring may be substituted by one or more ═O group. Most preferred heteroaryl groups that may be mentioned herein are 5- or 6-membered aromatic groups containing 1, 2 or 3 heteroatoms (e.g. preferably selected from nitrogen, oxygen and sulfur).

It may be specifically stated that the heteroaryl group is monocyclic or bicyclic. In the case where it is specified that the heteroaryl is bicyclic, then it may consist of a five-, six- or seven-membered monocyclic ring (e.g. a monocyclic heteroaryl ring) fused with another five-, six- or seven-membered ring (e.g. a monocyclic aryl or heteroaryl ring).

Heteroatoms that may be mentioned include phosphorus, silicon, boron and, preferably, oxygen, nitrogen and sulfur.

When “aromatic” groups are referred to herein, they may be aryl or heteroaryl. When “aromatic linker groups” are referred to herein, they may be aryl or heteroaryl, as defined herein, are preferably monocyclic (but may be polycyclic) and attached to the remainder of the molecule via any possible atoms of that linker group. However, when, specifically carbocylic aromatic linker groups are referred to, then such aromatic groups may not contain a heteroatom, i.e. they may be aryl (but not heteroaryl).

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

The invention may be described in several embodiments of the invention as follows:

R² is —C₁₋₄ alkyl optionally substituted by one or more substituents selected from halo;

R³ represents a substituent selected from H, F and —C₁₋₂ alkyl;

R⁴ is H, —R^{8a}, —C(═O)—R^{8b} or —SO₂—R⁹.

R^{5a} and R^{5b} independently represent hydrogen or —C₁₋₄ alkyl optionally substituted by one or more substituents selected from halo (e.g. F) and —O—CH₃;

R^{8a} represents —C₁₋₄ alkyl, optionally substituted by one or more substituents selected from halo, —OC₁₋₃ alkyl and —CN;

R^{8b} represents —C₁₋₃ alkyl (optionally substituted by one or more fluoro atoms);

R⁹ represents —N(R^{6c})R^{6d} or —C₁₋₄ alkyl optionally substituted by one or more substituents selected from halo (e.g. F) and —O—CH₃;

R^{6c} and R^{6d} independently represent H, C₁₋₆ alkyl or R^{6c} and R^{6d} are linked together to form a 3- to 6-membered ring;

R^{10a} and R^{10b} independently represent H, halo or C₁₋₄ alkyl;

R^{12a} and R^{12b} independently represent hydrogen or C₁₋₂ alkyl; or R^{12a} and R^{12b} are linked together to form a 3-membered ring; and/or

R^{12c} and R^{12d} independently represent hydrogen or C₁₋₂ alkyl; or R^{12c} and R^{12d} are linked together to form a 3-membered ring.

In an embodiment, ring A is represented as follow:

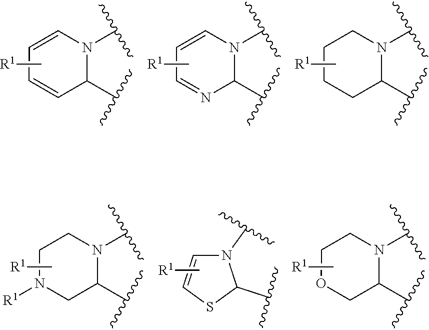

wherein R¹ represents one or more optional substituents as hereinbefore defined (and independently selected).

In another embodiment, ring A is represented as follow:

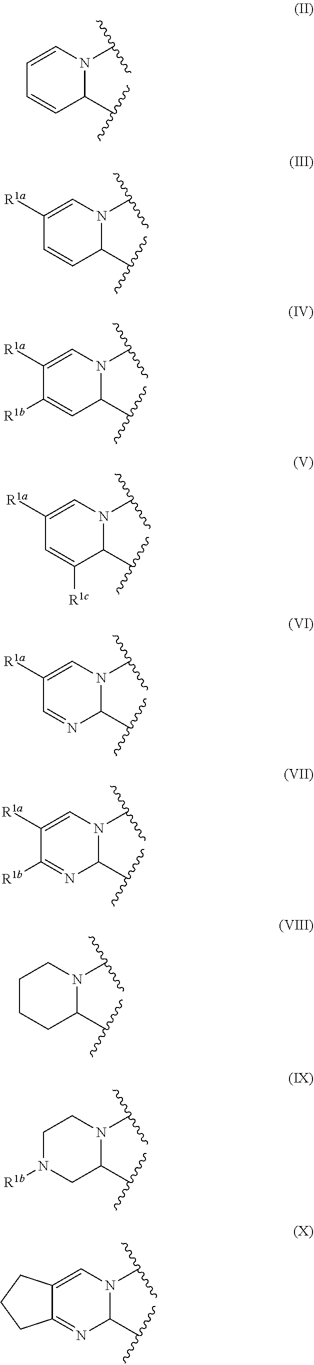

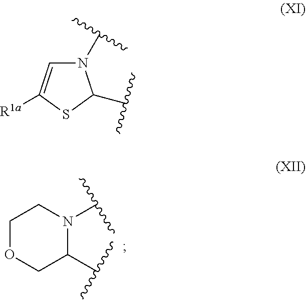

wherein R^{1a}, R^{1b} and R^{1c}, represent one or more R¹ optional substituents selected independently (and as hereinbefore defined).

In an embodiment, ring B represents a 5-membered ring containing two nitrogen atoms, and, in a specific embodiment, ring B represents the following:

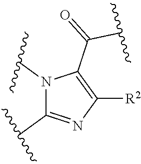

In an embodiment, the combined ring system, i.e. ring A and ring B may be represented as follow:

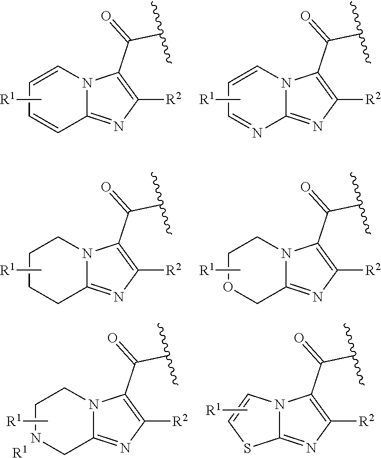

wherein R¹ represents one or more optional substituents as hereinbefore defined (and independently selected).

In another embodiment, the combined ring system, i.e. ring A and ring B may be represented as follow:

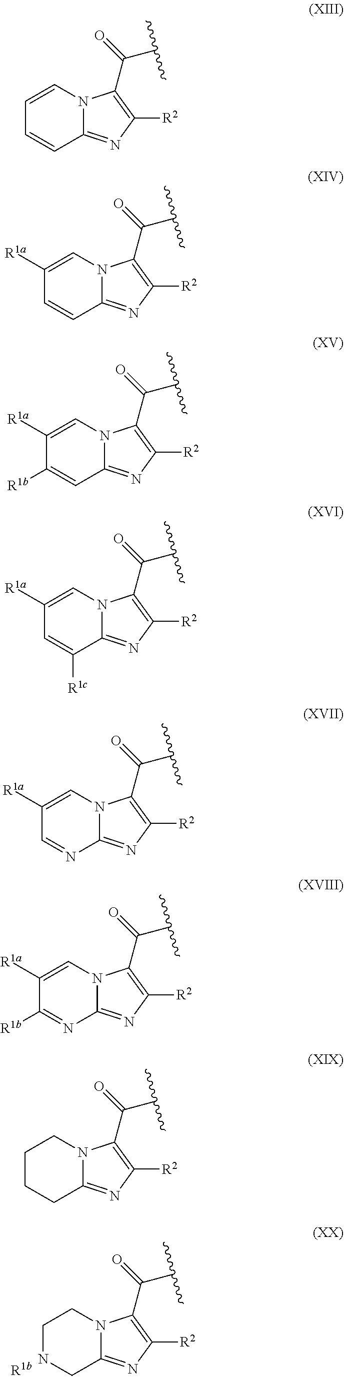

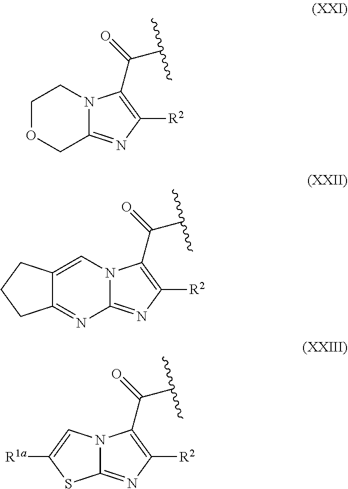

wherein R^{1a}, R^{1b} and R^{1c}, represent according to claim 1 the one or more R¹ optional substituents selected independently.

In an embodiment, ring C is represented as follow:

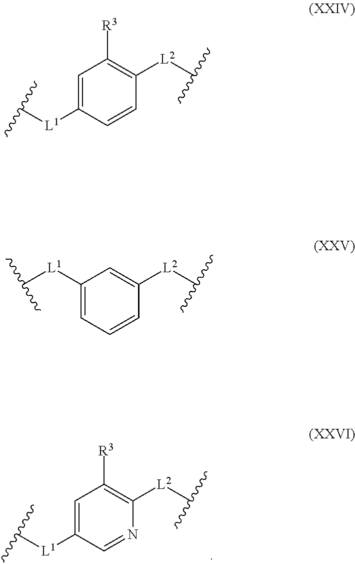

In an embodiment, ring C may also represent:

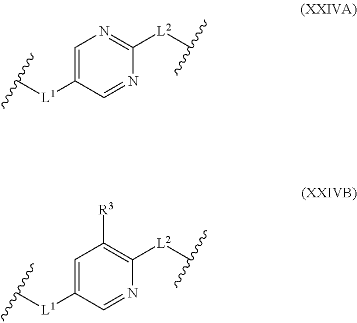

In an embodiment, the C ring may be optionally substituted for instance by R^{10a} and/or R³ (as defined herein). For instance, R^{10a} and/or R³ may represent halo (e.g. fluoro) or C₁₋₄ (e.g. C₁₋₂) alkyl (such as methyl). In this respect, we refer to the alternative embodiments that R³ and R^{10a} may be, as defined above (for instance, H, F, etc.).

In an embodiment, ring D is represented as follow:

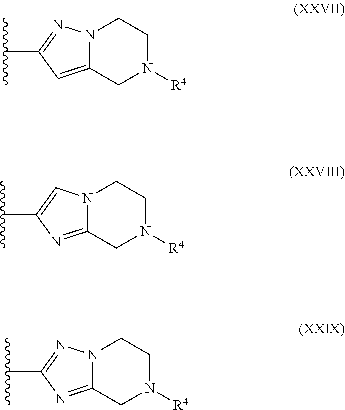

In an embodiment, the D ring (such as (XXVII), (XXVIII) and (XXIX) above) may be substituted, for instance by R^{10b}, in which R^{10b} is an herein defined (and for instance, specifically may represent C₁₋₄ alkyl, such as C₁₋₂ alkyl, e.g. methyl).

In an embodiment, L¹ represents a linker group, selected from: —CH₂—, —CH₂—CH₂—, —C(R^{12a})(R^{12b})—, and wherein R^{12a} and R^{12b} each independently represent —CH₃ or are linked together to form a 3-membered ring.

In an embodiment, L² represents a linker group, selected from: a direct bond, —CH₂—, a 4- or 5- or 6-membered non-aromatic ring optionally containing one or two nitrogen atom(s).

In an embodiment, R¹ (including R^{1a}, R^{1b} and/or R^{1c}) is not present or represents an optional substituent as defined herein (for instance, represents halo, e.g. chloro, or C₁₋₄ alkyl, such as methyl or ethyl).

In an embodiment, compounds of the invention include those in which:

R¹ represents one or more substituents selected from halo, C₁₋₄ alkyl, —OC₁₋₄ alkyl, —N(R^{6a})R^{6b}; or any two R¹ groups may be taken together (when attached to adjacent atoms of the A ring) to form a 5- or 6-membered ring optionally containing one or two heteroatoms, and which ring is optionally substituted by one or two C₁₋₃ alkyl substituents; and/or

R^{6a} and R^{6b} independently represent hydrogen or C₁₋₃ alkyl.

In another embodiment, compounds of the invention include those in which:

R¹ represents one or more substituents selected from halo (e.g. fluoro or chloro), C₁₋₄ alkyl (which may be straight-chain, so forming e.g. methyl or isopropyl, or, cyclic, so forming e.g. cyclopropyl), —OC₁₋₂ alkyl (so forming e.g. a —OCH₃ group), —NH₂, —N(H)(C₁₋₂ alkyl) (so forming e.g. a NHCH₃ group), or, two R¹ groups may be adjacent to each other and may be linked to form a 5- or 6-membered ring optionally containing one or two (e.g. one) heteroatom(s) (so forming e.g. a cyclopentyl moiety or a tetrahydropyranyl moiety).

In an embodiment, compounds of the invention include those in which:

R² represents C₁₋₃ alkyl optionally substituted by one or more fluoro atoms, so forming e.g. —CH₃, —CH₂CH₃, cyclopropyl, —CHF₂ or CF₃; and/or

L¹ represents —CH₂—, —CH₂CH₂—, —C(—CH₂—CH₂—)— or —C(CH₂)₂— (and in a specific embodiment, L¹ represents —CH₂—).

In an embodiment, X¹ represents ═C(R^{10a})— (in which R^{10a} represents H) and, in another embodiment, X¹ represents ═N—. In an embodiment, R³ represents H and, in another embodiment R³ represents fluoro.

In an embodiment, L² represents a direct bond (i.e. is not present) or represents a linker group selected from —CH₂— and a 4-6 membered heterocycloalkyl group containing one or two heteroatoms, so forming for example an azetidinyl linker group, a pyrrolidinyl linker group or a piperazinyl linker group; hence the linker groups, when cyclic groups, may represent:

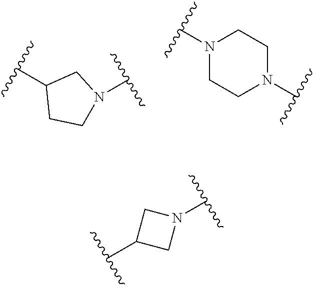 [SMILES: *C1CCN(*)C1 ; *N1CCN(*)CC1 ; *C1CN(*)C1]

In an embodiment, or in several embodiments:

R⁴ represents H, —R^{8a}, —C(═O)—R^{8b} or —SO₂—R⁹;

R^{8a} represents C₁₋₃ alkyl optionally substituted by one or two (e.g. one) substituent(s) selected from —OC₁₋₂ alkyl and —CN (so forming for example unsubstituted methyl or a —CH₂—CH₂—OCH₃ or —CH₂—CH₂—CN group);

R^{8b} represents C₁₋₃ alkyl (e.g. methyl);

R⁹ represents —N(R^{6c})R^{6d} or —C₁₋₄ alkyl optionally substituted by one or more fluoro atoms; and/or

R^{6c} and R^{6d} independently represent C₁₋₃ alkyl (e.g. methyl), or, are linked together to form a 3- to 6-membered ring (e.g. a 5-membered pyrrolidinyl ring).

In a particular embodiment R⁴ represents —SO₂—R⁹. In a further embodiment when R⁴ represents —SO₂—R⁹, then R⁹ represents C₁₋₂ alkyl optionally substituted by one or more fluoro atoms. In a specific embodiment, R⁴ represents —SO₂CF₃.

### Pharmacology

The compounds according to the invention have surprisingly been shown to be suitable for the treatment of a bacterial infection including a mycobacterial infection, particularly those diseases caused by pathogenic mycobacteria such as Mycobacterium tuberculosis (including the latent and drug resistant form thereof). The present invention thus also relates to compounds of the invention as defined hereinabove, for use as a medicine, in particular for use as a medicine for the treatment of a bacterial infection including a mycobacterial infection.

Such compounds of the invention may act by interfering with ATP synthase in M. tuberculosis, with the inhibition of cytochrome bc₁ activity being the primary mode of action. Cytochrome bc₁ is an essential component of the electron transport chain required for ATP synthesis.

Further, the present invention also relates to the use of a compound of the invention, as well as any of the pharmaceutical compositions thereof as described hereinafter for the manufacture of a medicament for the treatment of a bacterial infection including a mycobacterial infection.

Accordingly, in another aspect, the invention provides a method of treating a patient suffering from, or at risk of, a bacterial infection, including a mycobacterial infection, which comprises administering to the patient a therapeutically effective amount of a compound or pharmaceutical composition according to the invention.

The compounds of the present invention also show activity against resistant bacterial strains.

Whenever used hereinbefore or hereinafter, that the compounds can treat a bacterial infection it is meant that the compounds can treat an infection with one or more bacterial strains.

The invention also relates to a composition comprising a pharmaceutically acceptable carrier and, as active ingredient, a therapeutically effective amount of a compound according to the invention. The compounds according to the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

Depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

Given the fact that the compounds of formula (Ia) or Formula (Ib) are active against bacterial infections, the present compounds may be combined with other antibacterial agents in order to effectively combat bacterial infections.

Therefore, the present invention also relates to a combination of (a) a compound according to the invention, and (b) one or more other antibacterial agents.

The present invention also relates to a combination of (a) a compound according to the invention, and (b) one or more other antibacterial agents, for use as a medicine.

The present invention also relates to the use of a combination or pharmaceutical composition as defined directly above for the treatment of a bacterial infection.

A pharmaceutical composition comprising a pharmaceutically acceptable carrier and, as active ingredient, a therapeutically effective amount of (a) a compound according to the invention, and (b) one or more other antibacterial agents, is also comprised by the present invention.

The weight ratio of (a) the compound according to the invention and (b) the other antibacterial agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The compounds according to the invention and the one or more other antibacterial agents may be combined in a single preparation or they may be formulated in separate preparations so that they can be administered simultaneously, separately or sequentially. Thus, the present invention also relates to a product containing (a) a compound according to the invention, and (b) one or more other antibacterial agents, as a combined preparation for simultaneous, separate or sequential use in the treatment of a bacterial infection.

The other antibacterial agents which may be combined with the compounds of the invention are for example antibacterial agents known in the art. For example, the compounds of the invention may be combined with antibacterial agents known to interfere with the respiratory chain of Mycobacterium tuberculosis, including for example direct inhibitors of the ATP synthase (e.g. bedaquiline, bedaquiline fumarate or any other compounds that may have be disclosed in the prior art, e.g. compounds disclosed in WO2004/011436), inhibitors of ndh2 (e.g. clofazimine) and inhibitors of cytochrome bd. Additional mycobacterial agents which may be combined with the compounds of the invention are for example rifampicin (=rifampin); isoniazid; pyrazinamide; amikacin; ethionamide; ethambutol; streptomycin; para-aminosalicylic acid; cycloserine; capreomycin; kanamycin; thioacetazone; PA-824; delamanid; quinolones/fluoroquinolones such as for example moxifloxacin, gatifloxacin, ofloxacin, ciprofloxacin, sparfloxacin; macrolides such as for example clarithromycin, amoxycillin with clavulanic acid; rifamycins; rifabutin; rifapentin; as well as others, which are currently being developed (but may not yet be on the market; see e.g. http://www.newtbdrugs.org/pipeline.php).

Compounds of the invention (including forms and compositions/combinations comprising compounds of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. For instance compounds of the invention may advantages associated with: lower cardiotoxicity; no reactive metabolite formation (e.g. that may cause toxicity issues, e.g. genotoxicity); no formation of degradants (e.g. that are undesired or may elicit unwanted side-effects); and/or faster oral absorption and improved bioavailability.

### General Preparation

The compounds according to the invention can generally be prepared by a succession of steps, each of which may be known to the skilled person or described herein.

### Experimental Part

Compounds of formula I may be prepared in accordance with the techniques employed in the examples hereinafter (and those methods know by those skilled in the art), for example by using the following techniques.

Compounds of formula (I) may be prepared by:
(i) reaction of a compound of formula (XXX),

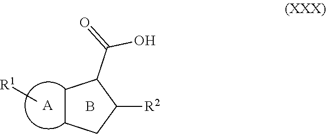

in which the integers are hereinbefore defined, with a compound of formula (XXXI),

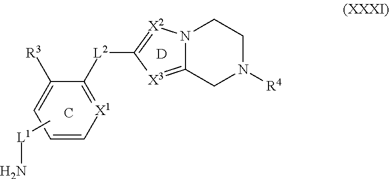

wherein the integers are as hereinbefore defined, which reaction may be performed in the presence of a suitable coupling reagent, for instance selected from diisopropylethylamine (DIPEA), 1-[bis(dimethylamino)methylene]-1H-1,2,3-triazolo[4,5-b]pyridinium-3-oxid hexafluorophosphate (HATU), 1-(3-dimethylaminopropyl)-3-ethylcarbodiimide (EDCI), 1-hydroxybenzotriazole (HOBt), O-(benzotriazole-1-yl)-N,N,N′,N′-tetramethyluronium tetrafluoroborate (TBTU), or a combination thereof, unders suitable conditions such as those described in the examples hereinafter; for example, in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof) or N,N′-disuccinimidyl carbonate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Alternatively, the carboxylic acid group of the compound of formula (XIV) may first be converted under standard conditions to the corresponding acyl chloride (e.g. in the presence of POCl₃, PCl₅, SOCl₂ or oxalyl chloride), which acyl chloride is then reacted with a compound of formula (XV), for example under similar conditions to those mentioned above;
(ii) coupling of a compound of formula (XXXII),

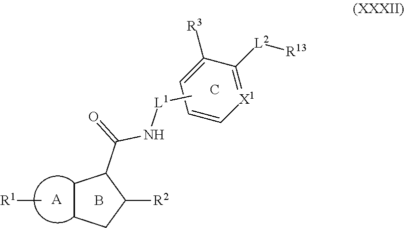

wherein the integers are as hereinbefore defined, and R¹³ represents a suitable group, e.g. a suitable leaving group such as chloro, bromo, iodo or a sulfonate group (for example a type of group that may be deployed for a coupling), with a compound of formula (XXXIII),

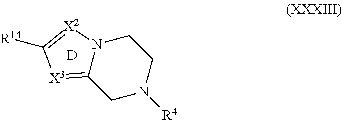

wherein R⁴ is as hereinbefore defined, and R¹⁴ represents a suitable group, e.g. a suitable leaving group under standard conditions, for example optionally in the presence of an appropriate metal catalyst (or a salt or complex thereof) such as Pd(dba)₂, Pd(OAc)₂, Cu, Cu(OAc)₂, CuI, NiCl₂ or the like, with an optional additive such as Ph₃P, X-phos or the like, in the presence of an appropriate base (e.g. t-BuONa, or the like) in a suitable solvent (e.g. dioxane or the like) under reaction conditions known to those skilled in the art.

It will be appreciated by those skilled in the art that some compounds of formula (I) may be converted to other compounds of formula (I).

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SCF).

The starting materials and the intermediates are compounds that are either commercially available or may be prepared according to conventional reaction procedures generally known in the art.

### EXAMPLES

### 1. General Information

### Melting Points

Melting points were recorded using a differential scanning calorimeter DSC 1 Mettler Toledo. Melting points were measured with a temperature gradient of 10° C. per min from 25 to 350° C. Values are peak values. Unless indicated, this method is used.

An alternative method is with open capilliary tubes on a Mettler Toledo MP50, which may be indicated at “MT”. With this method, melting points are measured with a temperature gradient of 10° C./minute. Maximum temperature is 300° C. The melting point data is read from a digital display and checked from a video recording system.

### ¹H NMR

¹H NMR spectra were recorded on a Bruker Avance DRX 400 spectrometer or Bruker Advance III 400 spectrometer using internal deuterium lock and equipped with reverse double-resonance (¹H, 13C, SEI) probe head with z gradients and operating at 400 MHz for proton and 100 MHz for carbon and a Bruker Avance 500 MHz spectrometer equipped with a Bruker 5 mm BBFO probe head with z gradients and operating at 500 MHz for proton and 125 MHz for carbon.

NMR spectra were recorded at ambient temperature unless otherwise stated.

Data are reported as follow: chemical shift in parts per million (ppm) relative to TMS (δ=0 ppm) on the scale, integration, multiplicity (s=singulet, d=doublet, t=triplet, q=quartet, quin=quintuplet, sex=sextuplet, m=multiplet, b=broad, or a combination of these), coupling constant(s) J in Hertz (Hz).

### HPLC-LCMS

### Analytical Methods

### LCMS

The mass of some compounds was recorded with LCMS (liquid chromatography mass spectrometry). The methods used are described below.

### General Procedure LCMS Methods A and B

The High Performance Liquid Chromatography (HPLC) measurement was performed using a LC pump, a diode-array (DAD) or a UV detector and a column as specified in the respective methods. If necessary, additional detectors were included (see table of methods below). Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used. Hereinafter, “SQD” means Single Quadrupole Detector, “RT” room temperature, “BEH” bridged ethylsiloxane/silica hybrid, “HSS” High Strength Silica, “DAD” Diode Array Detector, “MSD” Mass Selective Detector.

TABLE
LCMS Method codes (Flow expressed in mL/min; column
temperature (T) in ° C.; Run time in minutes).

| | | | | | Flow | |
|---|---|---|---|---|---|---|
| Method | | | | | Column T | Run |
| code | Instrument | Column | Mobile phase | gradient | 40 | time |
| A | Thermoscientific | Agilent: | A: HCOOH | 98% A for 2 min, | 1 | 18.4 |
| | Ultimate | Poroshell | 0.1% in | to 0% A in 10 | 30 | |
| | 3000 DAD | EC-C18 | water/ | min, held for 3.4 | | |
| | and | (4 μm, | B: HCOOH | min, back to 98% | | |
| | Brucker | 4.6 × 100 | 0.05% in | A in 1.3 min, | | |
| | HCT ultra | mm) | CH3CN | held for 1.7 min | | |
| B | Agilent | YMC- | A: 0.1% | From 95% A to | 2.6 | 6.2 |
| | 1100 | pack | HCOOH in | 5% A in 4.8 min, | 35 | |
| | HPLC | ODS- | H2O | held for 1.0 min, | | |
| | DAD | AQ C18 | B: CH3CN | to 95% A in 0.2 | | |
| | LC/MS | (50 × | | min. | | |
| | G1956A | 4.6 mm, | | | | |
| | | 3 μm) | | | | |

When a compound is a mixture of isomers which give different peaks in the LCMS method, only the retention time of the main component is given in the LCMS table.

### 2. Abbreviations (and Formulae)

AcOH Acetic acid
AcCl Acetyl chloride
BINAP 2,2′-bis(diphenylphosphino)-1,1′-binaphthyl
BrettPhos 2-(Dicyclohexylphosphino)3,6-dimethoxy-2′,4′,6′-triisopropyl-1,1′-biphenyl
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
CBr₄ Tetrabromomethane
CbzCl Benzyl chloroformate
CH₃CN/ACN Acetonitrile
Cs₂CO₃ Cesium carbonate
CSA Camphor-10-sulfonic acid
DCE Dichloroethane
DCM or CH₂Cl₂ Dichloromethane
DIPEA N,N-Diisopropylethylamine
DMAP 4-(Dimethylamino)pyridine
DME 1,2-Dimethoxyethane
DMF Dimethylformamide
DMF-DMA N,N-dimethylformamide dimethyl acetal
DMSO Methyl sulfoxide
EDCI·HCl N-(3-Dimethylaminopropyl)-N′-ethylcarbodiimide hydrochloride
Et₂O Diethylether
Et₃N or TEA Triethylamine
EtOAc Ethyl acetate
EtOH Ethanol
h hour
H₂ Dihydrogen gas
HATU Hexafluorophosphate Azabenzotriazole Tetramethyl Uronium
HBr Hydrobromic acid
HCl Hydrochloric acid
HFIP Hexafluoroisopropanol
HOBT·H₂O 1-Hydroxybenzotriazole hydrate
i-PrOH Isopropyl alcohol
K₂CO₃ Potassium carbonate
KHSO₄ Potassium bisulfate
LiBH₄ Lithium borohydride
LiOH Lithium hydroxide
LiHMDS Lithium bis(trimethylsilyl)amide
MeOH Methanol
MeI Iodomethane
MeTHF/2-MeTHF Methyltetrahydrofurane
MgSO₄ Magnesium sulfate
min Minute
N₂ Nitrogen
NaCl Sodium Chloride
NaHCO₃ Sodium Bicarbonate
NaNO₂ Sodium nitrite
NaOH Sodium hydroxide
NBS 1-bromopyrrolidine-2,5-dione
NH₃ Ammonia
NH₄Cl Ammonium, chloride
NH₄HCO₃ Ammonium bicarbonate
NMR Nuclear Magnetic Resonance
Pd/C Palladium on carbon
PdCl₂(PPh₃)₂ Dichlorobis(triphenylphosphine)palladium(II)
Pd(OAc)₂ Palladium(II) acetate
Pd₂dba₃ Tris(dibenzylideneacetone)dipalladium(O)
Pd(PPh₃)₄ Palladium-tetrakis(triphenylphosphine)
Pd(dffp)Cl₂·DCM [1,1′-Bis(diphenylphosphino)ferrocene]dichloropalladium(II) complex with dichloromethane
PIDA (Diacetoxyiodo)benzene
POCl₃ Phosphorous Oxychloride
Ra-Ni/Ni Raney Raney®-Nickel
rt/RT Room temperature
RuPhos 2-Dicyclohexylphosphino-2′,6′-diisopropoxybiphenyl
RuPhos Pd G3 (2-Dicyclohexylphosphino-2′,6′-diisopropoxy-1,1′-biphenyl)[2-(2′-amino-1,1′-biphenyl)] palladium(II) methanesulfonate
t-AmylOH tert-Amyl alcohol
SiOH Silica Gel
TBTU O-(benzotriazole-1-yl)-N,N,N′,N′-tetramethyluronium tetrafluoroborate
Tf₂O Trifluoromethanesulfonic Anhydride
TFA Trifluoroactetic acid
THF Tetrahydrofuran
TMSCl Trimethylsilyl chloride
TsOH or PTSA p-Toluensulfonic acid
AlMe₃ Trimethylaluminium
BH₃ 1M in THF Borane tetrahydrofuran complex solution 1.0 M in THF
CBrCl₃ Bromotrichloromethane
Boc₂O Di-tert-butyl dicarbonate
KOAc Potassium acetate
K3PO4·H2O Hydrated tripotassium phosphate
KHCO3 Potassium hydrogen carbonate

### Synthesis of Compound 1

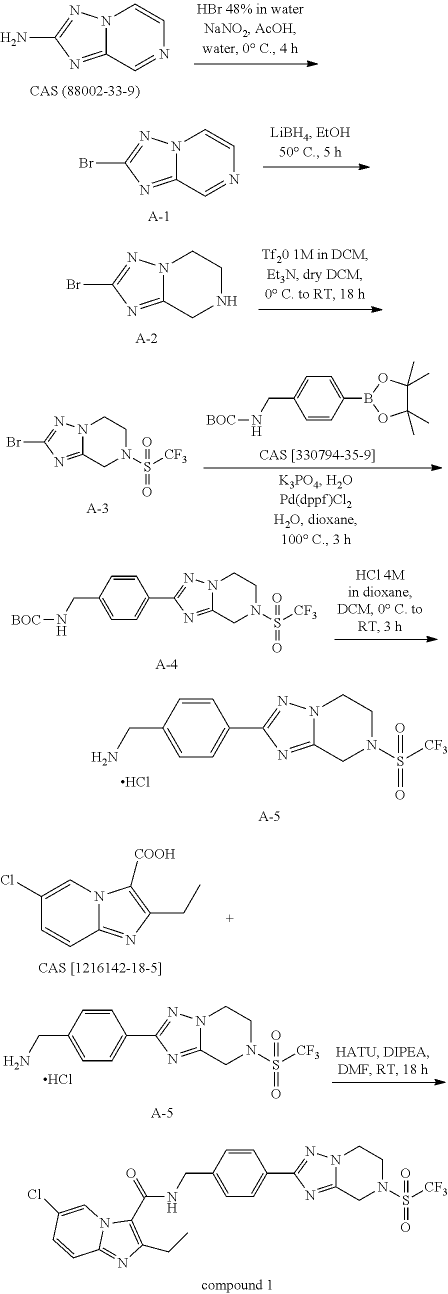

### Preparation of Compound A-1

To an argon-purged mixture of [1,2,4]Triazolo[1,5-a]pyrazin-2-amine (CAS [88002-33-9], 1.00 g, 7.40 mmol) in acetic acid (6.3 mL) were added successively Hydrobromic acid 48% in water (4.19 mL, 37.0 mmol) and a solution of Sodium nitrite (613 mg, 8.88 mmol, 1.2 eq.) in water (5.3 mL) at 0° C. The reaction mixture was stirred for 1 h at 0° C. A solution of sodium nitrite (511 mg, 7.40 mmol, 1 eq.) in water (4.4 mL) was added at 0° C. and the reaction mixture was stirred for 3 h at 0° C. The reaction mixture was concentrated under reduced pressure and partitioned between water (100 mL) and EtOAc (100 mL). The layers were separated, and the aqueous layer was extracted with EtOAc (2×100 mL). The combined organic layers were washed with brine (2×100 mL), dried over Na₂SO₄, filtered and concentrated under reduced pressure to afford an orange oil. This was purified by flash chromatography over silica gel (irregular SiOH, Cyclohexane/EtOAc from 100/0 to 50/50 over 55 min) to afford a white solid which was triturated with Et₂O (3 mL) to afford intermediate A-1 as a white solid, 0.63 g (43%).

### Preparation of Intermediate A-2

To an argon-purged mixture of A-1 (500 mg, 2.51 mmol) in Ethanol (22 mL) was added Lithium borohydride (219 mg, 10.0 mmol) at room temperature. The reaction mixture was stirred at 50° C. for 5 h. The reaction mixture was concentrated under reduced pressure and the resulting residue was quenched with a 1.0 M aq. HCl solution (pH ˜1, 30 mL) and extracted with EtOAc (2×100 mL). The aqueous layer was basified with a saturated aqueous Na₂CO₃ solution and extracted with DCM (3×100 mL). The combined organic layers were washed with brine (150 mL), dried over MgSO₄, filtered and concentrated under reduced pressure to afford intermediate A-2 as a white solid, 0.36 g (71%, the crude was used such as in the next step).

### Preparation of Intermediate A-3

To a solution of A-2 (340 mg, 1.68 mmol) and triethylamine (0.700 mL, 5.02 mmol) in DCM (10 mL) was added 1M Trifluoromethanesulfonic anhydride solution in DCM (3.35 mL, 3.35 mmol) dropwise at 0° C. The reaction mixture was allowed to warm to room temperature and stirred for 18 h. Water (15 mL) and DCM (15 mL) were added to the reaction mixture and the layers were separated. The organic layer was washed with brine, dried over MgSO₄, filtered and concentrated under reduced pressure to afford a brown gum. This was triturated with Et₂O (2×2 mL) to afford intermediate A-3 as a brown solid, 0.506 g (90%).

### Preparation of Intermediate A-4

An argon-purged mixture of A-3 (506 mg, 1.51 mmol), 4-(tert-Butoxycarbonylaminomethyl) phenylboronic acid, pinacol ester (604 mg, 1.81 mmol), potassium phosphate monohydrate (1.04 g, 4.53 mmol) and [1,1′-Bis(diphenylphosphino)ferrocene] dichloropalladium (111 mg, 0.151 mmol) in 1,4-dioxane (7.5 mL) and water (1.5 mL) was stirred at 100° C. for 3 h. The reaction mixture was cooled to room temperature, filtered on Celite® and washed with EtOAc (50 mL) to afford a brown gum. A purification was carried out by flash chromatography over silica gel (irregular SiOH, Cyclohexane/EtOAc from 100/0 to 50/50 over 50 min) to afford of intermediate A-4 as a yellowish solid, 0.51 g (73%).

### Preparation of Intermediate A-5

To an argon-purged mixture of A-4 (500 mg, 1.08 mmol) in DCM (2 mL) was added 4M solution of HCl in 1,4-dioxane (2.71 mL, 10.8 mmol) at 0° C. The reaction mixture was allowed to warm to room temperature and stirred for 3 h. The reaction mixture was concentrated under reduced pressure to afford intermediate A-5 as a white solid, 0.42 g (97%).

### Preparation of Compound 1

To an argon-purged mixture of 6-Chloro-2-ethylimidazo[1,2-a]pyridine-3-carboxylic acid (CAS [1216142-18-5], 99.8 mg, 0.444 mmol) in DMF (6 mL) was added HATU (203 mg, 0.533 mmol) at room temperature. The reaction mixture was stirred for 5 minutes at room temperature before the addition of A-5 (212 mg, 0.533 mmol) and DIPEA (0.309 mL, 1.78 mmol). The resulting mixture was stirred at room temperature for 16 h and poured into water (20 mL). The resulting precipitate was filtered on a glass-frit, washed with water (3×20 mL) and dried under vacuum at 60° C. to afford a brown solid. A purification was carried out by flash chromatography over silica gel (irregular SiOH, DCM/MeOH from 100/0 to 95/5 over 45 min) to afford a brownish solid. This was triturated with MeOH (2 mL) and vacuum-dried at 60° C. for 48 h to afford compound 1 as a beige solid, 0.12 g (48%).

¹H NMR (400 MHz, DMSO-d6) δ ppm 9.10 (d, J=1.7 Hz, 1H), 8.53 (t, J=5.9 Hz, 1H), 7.97 (d, J=8.2 Hz, 2H), 7.68 (d, J=9.5 Hz, 1H), 7.48 (d, J=8.2 Hz, 2H), 7.47 (dd, J=9.5 Hz, 2.1 Hz, 1H), 4.96 (s, 2H), 4.59 (d, J=5.9 Hz, 2H), 4.36 (t, J=5.4 Hz, 2H), 4.15 (t, J=5.2 Hz, 2H), 3.02 (q, J=7.5 Hz, 2H), 1.27 (t, J=7.5 Hz, 3H).

### Synthesis of Compound 2

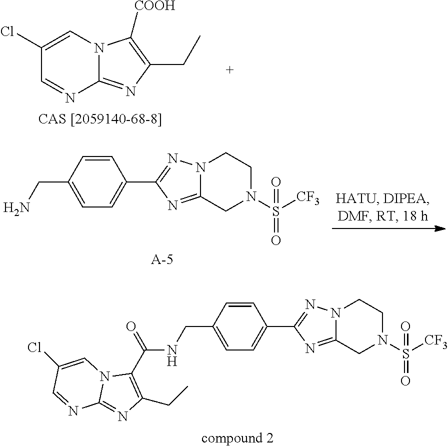 [SMILES: CCc1nc2ncc(Cl)cn2c1C(=O)O ; NCc1ccc(-c2nc3n(n2)CCN(S(=O)(=O)C(F)(F)F)C3)cc1 ; "A-5" ; CCc1nc2ncc(Cl)cn2c1C(=O)NCc1ccc(-c2nc3n(n2)CCN(S(=O)(=O)C(F)(F)F)C3)cc1]

Accordingly, compound 2 was prepared in the same way as compound 1 starting from 6-Chloro-2-ethylimidazo[1,2-a]pyrimidine-3-carboxylic acid CAS [2059140-68-8] (0.39 mmol) and intermediate A-5 (0.46 mmol) yielding 0.13 g (57%) as a white powder.

¹H NMR (400 MHz, DMSO-d6) δ ppm 9.43 (d, J=2.6 Hz, 1H), 8.69 (d, J=2.6 Hz, 1H), 8.63 (t, J=6.0 Hz, 1H), 7.97 (d, J=8.1 Hz, 2H), 7.48 (d, J=8.1 Hz, 2H), 4.96 (s, 2H), 4.59 (d, J=5.9 Hz, 2H), 4.36 (t, J=5.3 Hz, 2H), 4.15 (t, J=5.3 Hz, 2H), 3.05 (q, J=7.5 Hz, 2H), 1.29 (t, J=7.5 Hz, 3H).

### Synthesis of Compound 4

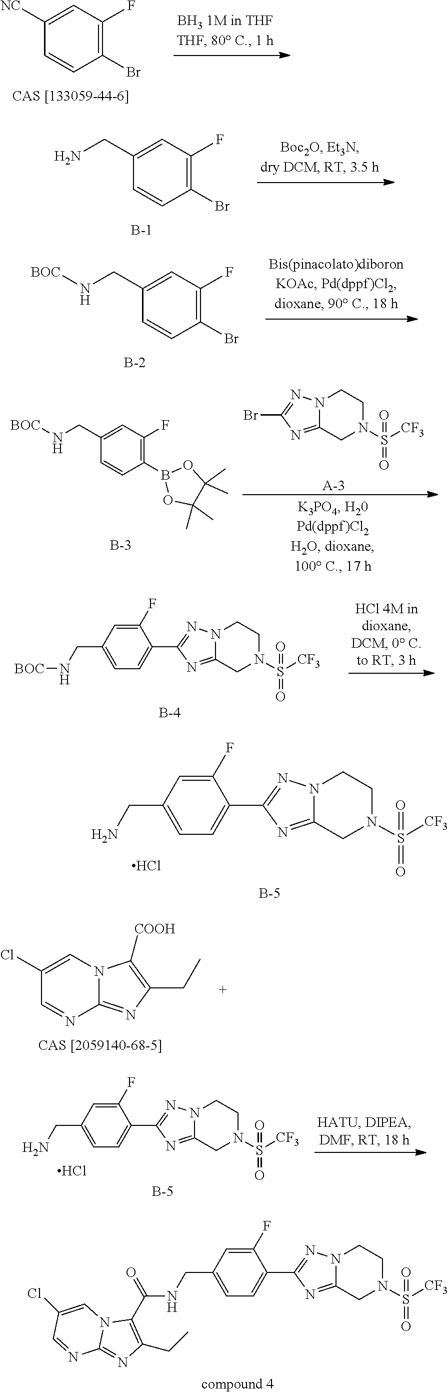

### Preparation of intermediate B-1

To a solution of 4-Bromo-3-fluorobenzonitrile (CAS [133059-44-6], 2.00 g, 10.0 mmol) in THF (8 mL) was added Borane tetrahydrofuran complex in THF (1M) (30.0 mL, 30.0 mmol) at room temperature. The reaction mixture was stirred at 80° C. for 1 h. The reaction mixture was quenched with MeOH (20 mL) and stirred for 10 min, then concentrated under reduced pressure to afford a yellow oil, 2.51 g (quantitative), used as such without further purifications.

### Preparation of Intermediate B-2

To a solution of B-1 (2.40 g, 9.56 mmol) and triethylamine (4.00 mL, 28.7 mmol) in DCM (60 mL) was added Di-tert-butyl dicarbonate (2.19 g, 10.0 mmol) at 15° C., then the reaction mixture was stirred at room temperature for 3.5 h. The reaction mixture was concentrated under reduced pressure to afford a sticky oil (3.7 g). The crude was purified by flash chromatography over silica gel (irregular SiOH, eluent: from 4 to 36% of EtOAc in Cyclohexane) affording intermediate B-2 after vacuum-drying at 60° C. for 17 h as a white solid, 2.17 g (75%).

### Preparation of Intermediate B-3

To a N₂ purged solution of B-2 (1.92 g, 6.31 mmol), Bis(pinacolato)diboron (1.92 g, 7.58 mmol) and Potassium acetate (1.55 g, 15.8 mmol) in 1,4-dioxane (31 mL) was added [1,1′-Bis(diphenylphosphino)ferrocene] dichloropalladium(II) (462 mg, 0.631 mmol), then the reaction mixture was stirred at 90° C. for 18 h. The reaction mixture was filtered on Celite®, the filter cake was rinsed with EtOAc (˜20 mL) and the filtrate was concentrated under reduced pressure. The residue was purified by flash chromatography over silica gel (irregular SiOH, 3 to 30% of EtOAc in Cyclohexane) to afford intermediate B-3 as a colourless oil which crystalized on standing over time as a white solid, 1.3 g (60%).

### Preparation of Intermediate B-4

A N²-purged mixture of intermediate A-3 (300 mg, 0.895 mmol), B-3 (377 mg, 1.07 mmol), Potassium phosphate tribasic monohydrate (618 mg, 2.69 mmol) and 1,1′-Bis(diphenylphosphino) ferrocene dichloropalladium (II) (65.5 mg, 0.090 mmol) in 1,4-dioxane (3.6 mL) and water (0.9 mL) was stirred at 100° C. for 17 h. The reaction mixture was cooled to rt, filtered on Celite® and the filter cake was washed with EtOAc (50 mL). The filtrate was concentrated and purified by flash chromatography over silica gel (irregular SiOH, Cyclohexane/EtOAc from 94/6 to 50/50 over 30 min) to afford intermediate B-4 as a white solid, 0.28 g (65%).

### Preparation of Intermediate B-5

To a nitrogen-purged mixture of B-4 (280 mg, 0.584 mmol) in dry DCM (1.2 mL) was added 4M solution of HCl in 1,4-dioxane (1.46 mL, 5.84 mmol) at 0° C. The reaction mixture was allowed to warm to room temperature and stirred for 3 h. The reaction mixture was concentrated under reduced pressure to afford intermediate B-5 as a white solid, 0.243 g (quantitative).

### Preparation of Compound 4

Accordingly, compound 4 was prepared in the same way as compound 1 starting from 6-Chloro-2-ethylimidazo[1,2-a]pyrimidine-3-carboxylic acid CAS [2059140-68-8] (0.4 mmol) and intermediate B-5 (0.48 mmol) yielding 0.13 g (57%) as a white powder.

¹H NMR (500 MHz, DMSO-d6) δ ppm 9.44 (d, J=2.7 Hz, 1H), 8.70 (d, J=2.7 Hz, 1H), 8.65 (t, J=6.0 Hz, 1H), 7.97 (t, J=8.0 Hz, 1H), 7.37-7.31 (m, 2H), 4.97 (s, 2H), 4.60 (d, J=5.9 Hz, 2H), 4.39 (t, J=5.5 Hz, 2H), 4.16 (t, J=5.0 Hz, 2H), 3.06 (q, J=7.5 Hz, 2H), 1.30 (t, J=7.5 Hz, 3H).

### Synthesis of Compound 7

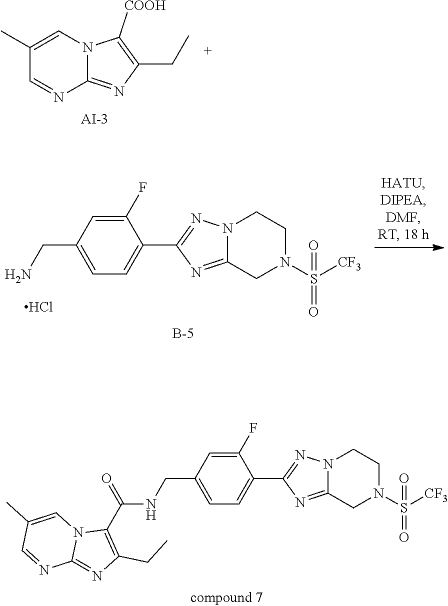

Accordingly, compound 7 was prepared in the same way as compound 1 starting from intermediate AI-3 (0.72 mmol) and intermediate B-5 (0.45 mmol) yielding 0.084 g (32%) as a white powder.

1H NMR (400 MHz, DMSO) δ 9.22-9.13 (m, 1H), 8.56-8.46 (m, 2H), 7.96 (t, J=7.8 Hz, 1H), 7.36-7.27 (m, 2H), 4.97 (s, 2H), 4.59 (d, J=5.9 Hz, 2H), 4.38 (t, J=5.4 Hz, 2H), 4.16 (t, J=5.2 Hz, 2H), 3.03 (q, J=7.5 Hz, 2H), 2.34 (s, 3H), 1.29 (t, J=7.5 Hz, 3H).

### Synthesis of Compound 8

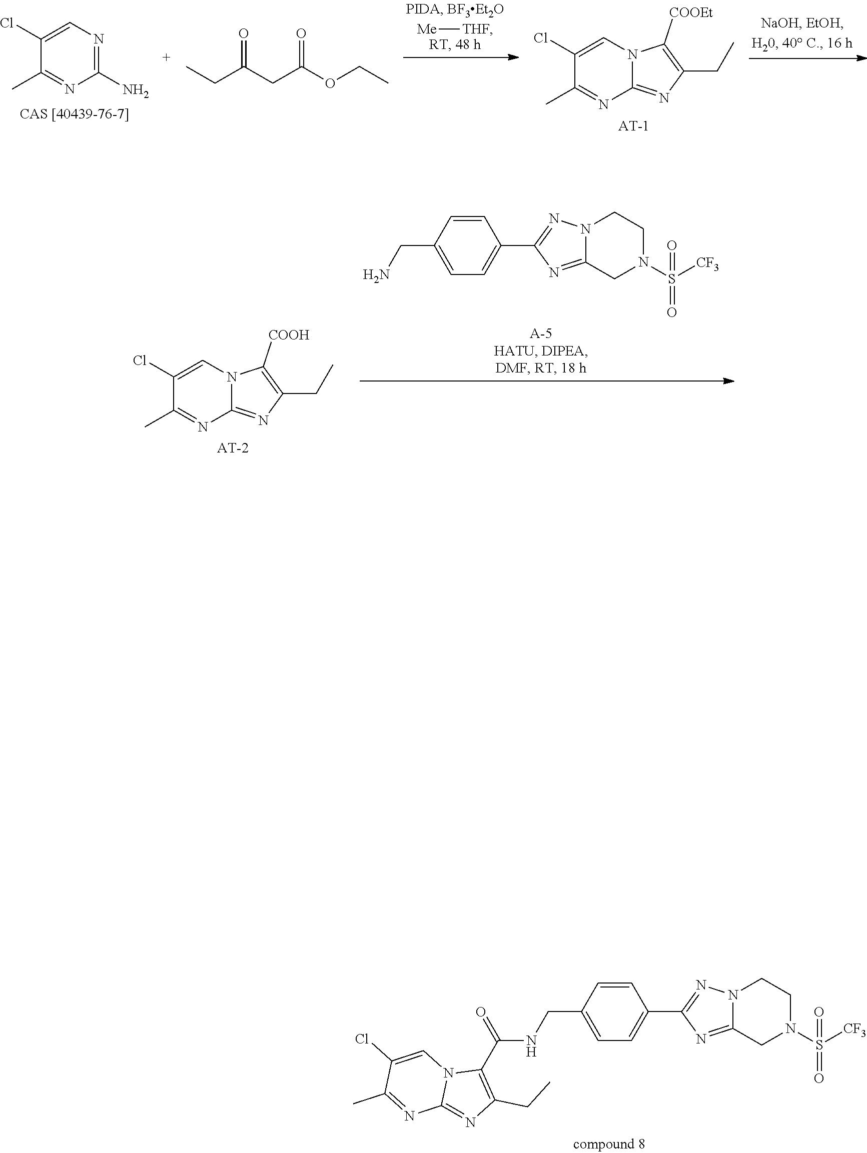

### Preparation of Intermediate AT-1

To a solution of 5-chloro-4-methylpyrimidin-2-amine (CAS [40439-76-7], 1 g, 6.97 mmol) in Me-THF (33 mL) at 0° C. were added iodobenzene diacetate (2.24 g, 6.96 mmol) and ethyl 3-oxovalerate (1.66 mL, 11.6 mmol). Then boron trifluoride etherate (91.3 μL, 0.349 mmol) was added dropwise. The solution was stirred at 5° C. for 1 h and then at room temperature for 18 h. EtOAc and water were added. The organic layer was washed with brine, dried (MgSO₄), evaporated and purified by preparative LC (irregular SiOH, 15-40 μm, 80 g, liquid loading (DCM) mobile phase gradient: from heptane/EtOAc 80:20 to 0:100 over 10 CV) the fractions containing product were evaporated to afford 367 mg of intermediate AT-1

### Preparation of Intermediate AT-2

A mixture of AT-1 (100 mg, 0.374 mmol), NaOH (45 mg, 1.12 mmol) and EtOH (2 mL) was stirred at room temperature for 2 days. The mixture was evaporated to give 164 mg of intermediate AT-2 (purity was estimated to give a quantitative yield).

### Preparation of Compound 8

Accordingly, compound 8 was prepared in the same way as compound 7 starting from intermediate AT-2 (0.45 mmol) and intermediate A-5 (0.37 mmol) affording 0.09 g (40%) as white powder.

¹H NMR (400 MHz, DMSO) δ 9.37 (s, 1H), 8.53 (brs, 1H), 7.96 (t, J=8.0 Hz, 1H), 7.35-7.30 (m, 2H), 4.97 (s, 2H), 4.59 (s, 2H), 4.38 (t, J=5.4 Hz, 2H), 4.16 (t, J=5.3 Hz, 2H), 3.03 (q, J=7.5 Hz, 2H), 2.62 (s, 3H), 1.28 (t, J=7.5 Hz, 3H).

### Synthesis of Compound 21

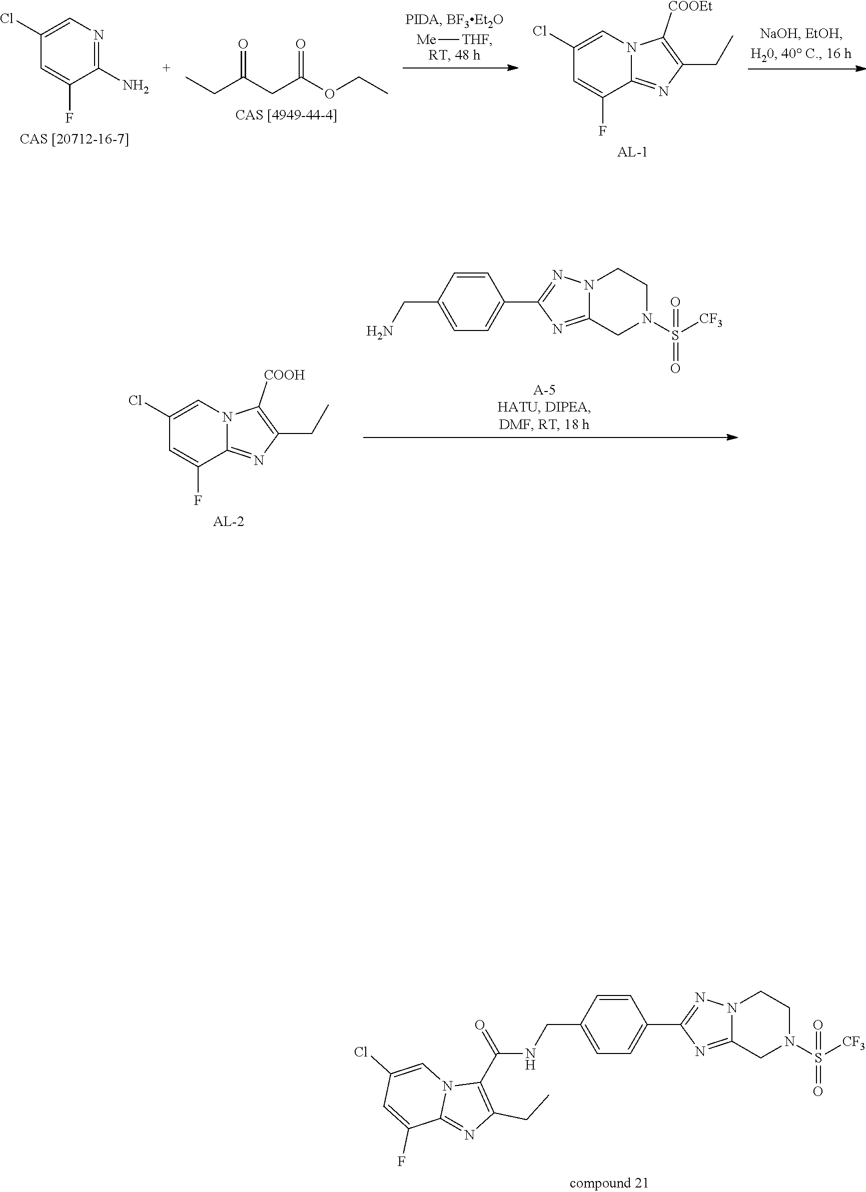

### Preparation of Intermediate AL-1

To a solution of 2-Amino-5-chloro-3-fluoropyridine (CAS [20712-16-7], 2.50 g, 17.1 mmol) in 2-MeTHF (75 mL) at 5° C. under N₂ were added Ethyl propionylacetate (2.5 mL, 17.6 mmol), Iodobenzene diacetate (5.50 g, 17.1 mmol) and Boron trifluoride diethyl etherate (105 μL, 0.851 mmol) the reaction was stirred at 5° C. for 30 min then at room temperature for 18 h. An extra amount of Ethyl propionylacetate (1.25 mL, 8.77 mmol), Iodobenzene diacetate (2.75 g, 8.54 mmol) and Boron trifluoride diethyl etherate (105 μL, 0.851 mmol) were added and the mixture was stirred at room temperature for 48 h. EtOAc (150 mL) and water (150 mL) were added. The layers were separated, and the organic layer was washed with a saturated aqueous solution of NaHCO₃ (200 mL), brine (2×200 mL), dried over Na₂SO₄, filtered and evaporated to afford 8.40 g as a brown viscous oil. This one was purified via preparative LC (SiOH, 120 g, 50 μm, Eluent: Cyclohexane/EtOAc, from 100:00 to 50:50), fractions containing product were collected and evaporated to afford 520 mg of intermediate AJ-1 as an orange paste (11%).

### Preparation of Intermediate AL-2

To a solution of AL-1 (480 mg, 1.77 mmol) in water (9 mL) and EtOH (9 mL) was added NaOH (213 mg, 5.33 mmol). The reaction mixture was stirred for 3 h at 30° C. The crude was washed with DCM (30 mL) and with EtOAc (30 mL), the aqueous phase was acidified with an aqueous solution of HCl (3N) until pH=2, extracted with DCM (2×50 mL). The layers were separated, and the organic layer was dried over Na₂SO₄, filtered and evaporated to afford 260 mg of intermediate AL-2 as a pale pink solid (60%).

### Preparation of Compound 21

Accordingly, compound 21 was prepared in the same way as compound 7 starting from intermediate AL-2 (0.72 mmol) and intermediate A-5 (0.45 mmol) affording 0.084 g (32%) as white solid.

¹H NMR (400 MHz, DMSO) δ 9.22-9.13 (m, 1H), 8.56-8.46 (m, 2H), 7.96 (t, J=7.8 Hz, 1H), 7.36-7.27 (m, 2H), 4.97 (s, 2H), 4.59 (d, J=5.9 Hz, 2H), 4.38 (t, J=5.4 Hz, 2H), 4.16 (t, J=5.2 Hz, 2H), 3.03 (q, J=7.5 Hz, 2H), 2.34 (s, 3H), 1.29 (t, J=7.5 Hz, 3H).

### Synthesis of Compound 22

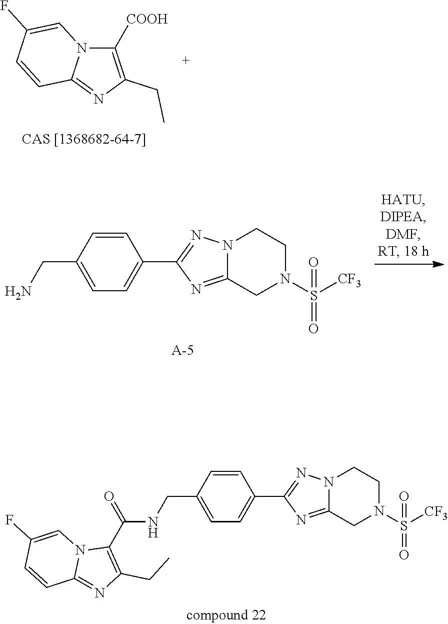 [SMILES: CCc1nc2ccc(F)cn2c1C(=O)O ; NCc1ccc(-c2nc3n(n2)CCN(S(=O)(=O)C(F)(F)F)C3)cc1 ; "A-5" ; CCc1nc2ccc(F)cn2c1C(=O)NCc1ccc(-c2nc3n(n2)CCN(S(=O)(=O)C(F)(F)F)C3)cc1]

Accordingly, compound 22 was prepared in the same way as compound 7 starting from 2-ethyl-6-fluoroimidazo[1,2-a]pyridine-3-carboxylic acid (CAS [1368682-64-7], 0.41 mmol) and intermediate A-5 (0.33 mmol) affording 0.084 g (46%) as white solid.

¹H NMR (400 MHz, DMSO) δ 9.10-9.03 (m, 1H), 8.48 (t, J=6.0 Hz, 1H), 7.98 (d, J=8.2 Hz, 2H), 7.70 (dd, J=9.8, 5.4 Hz, 1H), 7.53-7.46 (m, 3H), 4.97 (s, 2H), 4.60 (d, J=5.9 Hz, 2H), 4.37 (t, J=5.4 Hz, 2H), 4.25-4.08 (m, 2H), 3.03 (q, J=7.5 Hz, 2H), 1.28 (t, J=7.5 Hz, 3H).

### Synthesis of Compound 23

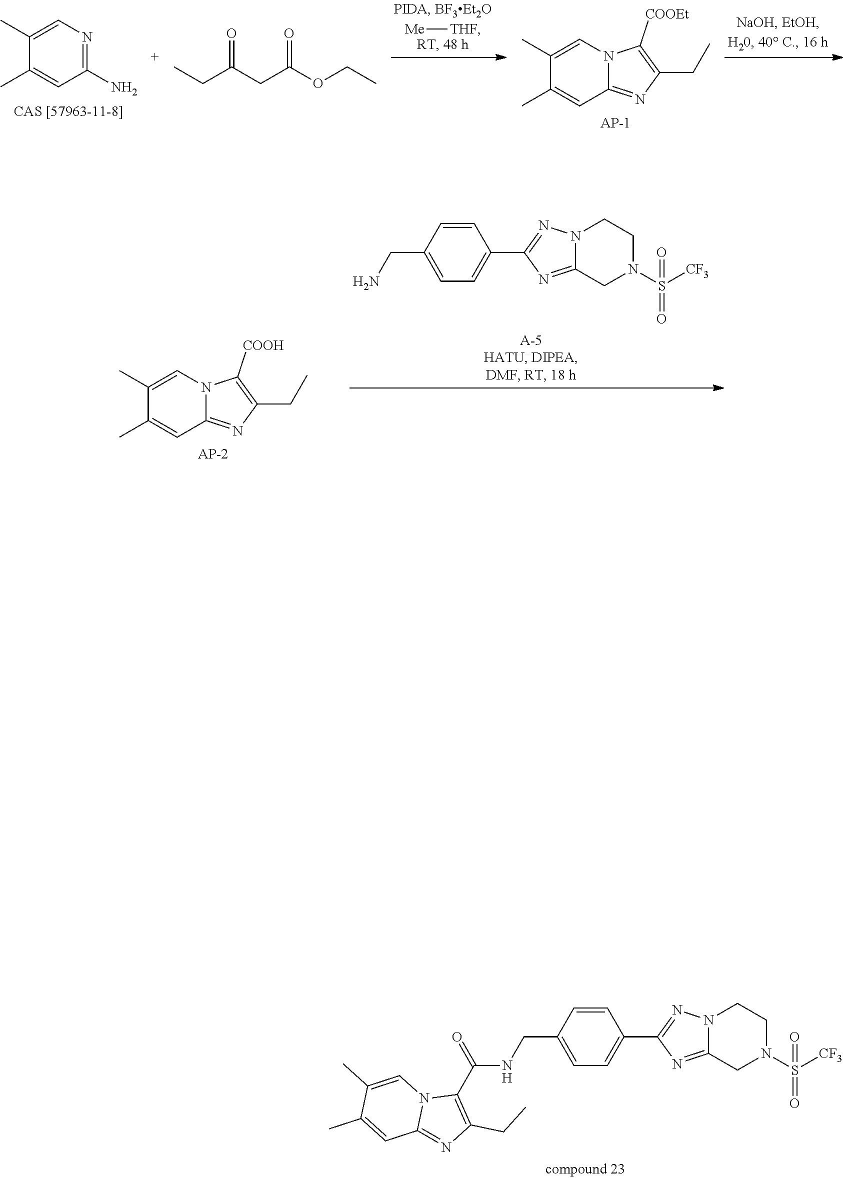

### Preparation of Intermediate AP-1

Accordingly, intermediate AP-1 was prepared in the same way as AL-1 starting from 4,5-dimethylpyridin-2-amine (CAS [57963-11-8], 4.09 mmol) and ethyl 3-oxovalerate (CAS [4949-44-4]) giving 0.73 g (72%) as white solid.

### Preparation of Intermediate AP-2

Accordingly, intermediate AP-2 was prepared in the same way as intermediate AL-2 starting from intermediate AP-1 (0.81 mmol) giving 0.3 g (quantitative).

### Preparation of Compound 23

Accordingly, compound 23 was prepared in the same way as compound 7 starting from intermediate AP-2 (0.46 mmol) and intermediate A-5 (0.36 mmol) affording 0.110 g (54%) as white powder.

¹H NMR (400 MHz, DMSO) δ 8.80 (s, 1H), 8.30 (t, J=6.0 Hz, 1H), 7.97 (d, J=8.2 Hz, 2H), 7.46 (d, J=8.2 Hz, 2H), 7.38 (s, 1H), 4.96 (s, 2H), 4.57 (d, J=5.9 Hz, 2H), 4.36 (t, J=5.4 Hz, 2H), 4.15 (t, J=5.2 Hz, 2H), 2.97 (q, J=7.5 Hz, 2H), 2.31 (s, 3H), 2.22 (s, 3H), 1.25 (t, J=7.5 Hz, 3H).

### Synthesis of Compound 24

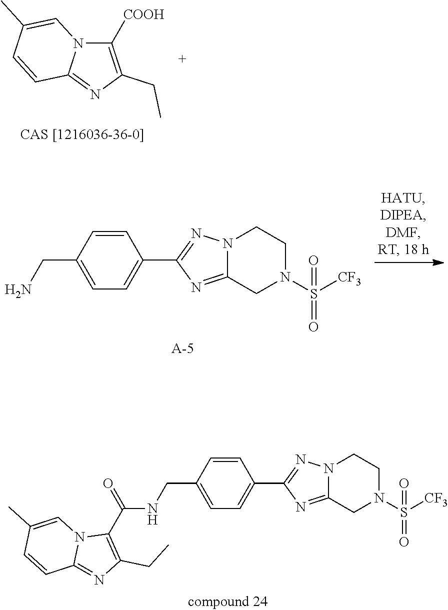 [SMILES: CCc1nc2ccc(C)cn2c1C(=O)O ; NCc1ccc(-c2nc3n(n2)CCN(S(=O)(=O)C(F)(F)F)C3)cc1 ; "A-5" ; CCc1nc2ccc(C)cn2c1C(=O)NCc1ccc(-c2nc3n(n2)CCN(S(=O)(=O)C(F)(F)F)C3)cc1]

Accordingly, compound 24 was prepared in the same way as compound 7 starting from 2-ethyl-6-methylimidazo[1,2-a]pyridine-3-carboxylic acid (CAS [1216036-36-0], 0.43 mmol) and intermediate AA-3 (0.33 mmol) affording 0.111 g (61%) as a white solid.

¹H NMR (400 MHz, DMSO-d6) δ 8.81 (s, 1H), 8.41 (t, J=5.9 Hz, 1H), 7.97 (d, J=8.1 Hz, 2H), 7.52 (d, J=9.1 Hz, 1H), 7.47 (d, J=8.2 Hz, 2H), 7.25 (dd, J=9.1, 1.3 Hz, 1H), 4.96 (s, 2H), 4.58 (d, J=5.9 Hz, 2H), 4.36 (t, J=5.4 Hz, 2H), 4.15 (t, J=5.1 Hz, 2H), 2.98 (q, J=7.5 Hz, 2H), 2.31 (s, 3H), 1.26 (t, J=7.5 Hz, 3H).

### Synthesis of Compound 36

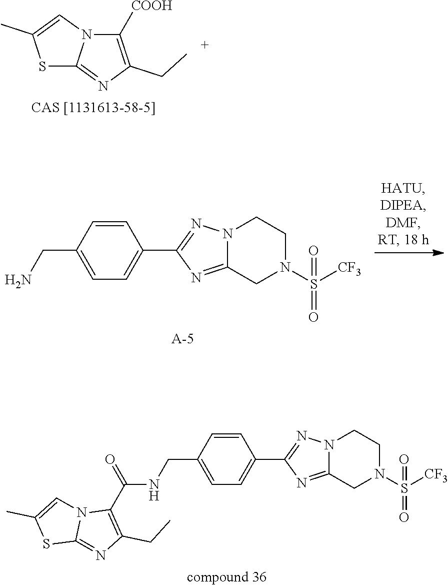 [SMILES: CCc1nc2sc(C)cn2c1C(=O)O ; NCc1ccc(-c2nc3n(n2)CCN(S(=O)(=O)C(F)(F)F)C3)cc1 ; "A-5" ; CCc1nc2sc(C)cn2c1C(=O)NCc1ccc(-c2nc3n(n2)CCN(S(=O)(=O)C(F)(F)F)C3)cc1]

Accordingly, compound 36 was prepared in the same way as compound 7 starting from 6-ethyl-2-methylimidazo[2,1-b][1,3]thiazole-5-carboxylic acid (CAS [1131613-58-5], 0.41 mmol) and intermediate A-5 (0.33 mmol) yielding 0.124 g (68%) as a white powder.

¹H NMR (400 MHz, DMSO) δ 8.19 (t, J=6.0 Hz, 1H), 7.96 (d, J=8.2 Hz, 2H), 7.92 (d, J=1.4 Hz, 1H), 7.44 (d, J=8.2 Hz, 2H), 4.96 (s, 2H), 4.54 (d, J=5.9 Hz, 2H), 4.42-4.31 (m, 2H), 4.24-4.10 (m, 2H), 2.90 (q, J=7.5 Hz, 2H), 2.42 (d, J=1.0 Hz, 3H), 1.23 (t, J=7.5 Hz, 3H).

### Synthesis of Compound 28

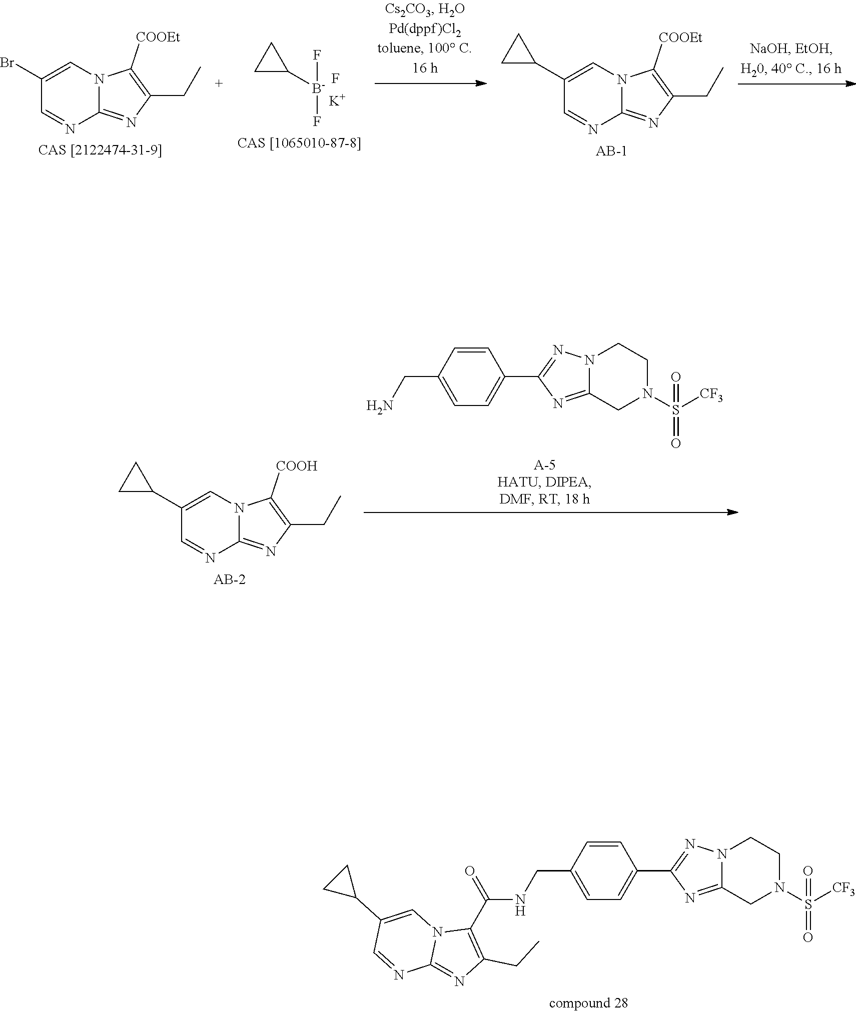

### Preparation of Intermediate AB-1

Potassium cyclopropyltrifluoroborate (0.62 g, 4.19 mmol), cesium carbonate (1.2 g, 3.69 mmol) and Pd(dppf)Cl₂ (0.2 g, 0.25 mmol) were added to a solution of ethyl 6-bromo-2-ethylimidazo[1,2-a]pyrimidine-3-carboxylate (CAS [2142474-31-9] in toluene (25 mL) and water (10 mL) a screw top vial while N₂ was bubbling at rt. The mixture was stirred at 100° C. for 16 h. Water was added, and the mixture was extracted with ethyl acetate. The combined organic layers were dried over MgSO₄, filtered and concentrated in vacuo. The crude product was purified by flash column chromatography (SiOH; ethyl acetate in heptane, from 0/100 to 50/50). The desired fractions were collected and concentrated in vacuo to yield intermediate AB-1 as a brown solid (0.35 g, 76%).

### Preparation of Compound AB-2

Accordingly, intermediate AB-2 was prepared in the same way as intermediate AL-2 starting from intermediate AB-1 (0.58 mmol) giving 0.17 g (quantitative).

### Preparation of Compound 28

Accordingly, compound 28 was prepared in the same way as compound 7 starting from intermediate AB-2 (0.58 mmol) and intermediate A-5 (0.37 mmol) yielding 0.17 g (77%) as a white solid.

¹H NMR (400 MHz, DMSO) δ 9.06 (d, J=2.4 Hz, 1H), 8.51 (t, J=5.9 Hz, 1H), 8.46 (d, J=2.5 Hz, 1H), 7.97 (d, J=8.2 Hz, 2H), 7.47 (d, J=8.2 Hz, 2H), 4.96 (s, 2H), 4.58 (d, J=5.9 Hz, 2H), 4.36 (t, J=5.4 Hz, 2H), 4.23-4.07 (m, 2H), 3.02 (q, J=7.5 Hz, 2H), 2.13-2.02 (m, 1H), 1.27 (t, J=7.5 Hz, 3H), 1.04-0.97 (m, 2H), 0.82-0.74 (m, 2H).

### Synthesis of Compound 29

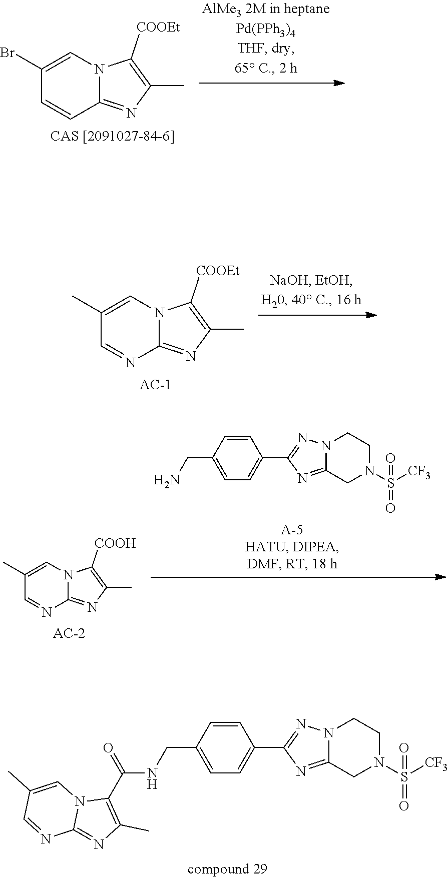

### Preparation of Intermediate AC-1

Trimethylaluminum solution 2M in heptane (2.54 mL, 5.08 mmol) was added dropwise to a solution of ethyl 6-bromo-2-methylimidazo[1,2-a]pyrimidine-3-carboxylate (CAS [2091027-34-6], 0.41 g, 1.12 mmol) and Pd(PPh₃)₄ (0.084 g, 0.073 mmol) in THF dry (11 mL) in a round bottom flask 2-neck charged with a condenser under nitrogen atmosphere at room temperature. Then the mixture was stirred at 65° C. for 2 h. The mixture was cooled to 0° C. and diluted with DCM. Then 10 ml of water was added dropwise. Then MgSO₄ powder was added and the mixture was stirred at room temperature for 30 min. The result was filtered through of pad of Celite®, washed with ethyl acetate and concentrated in vacuo. The crude product was purified by flash column chromatography (SiOH, 25 g; DCM/MeOH (9:1) in DCM 0/100 to 20/80). The desired fractions were collected and concentrated in vacuo to yield intermediate AC-1 as a yellow solid (0.19 g, 59%).

### Preparation of Intermediate AC-2

Accordingly, intermediate AC-2 was prepared in the same way as intermediate AL-2 starting from intermediate AC-1 (0.68 mmol) giving 0.14 g (quantitative).

### Preparation of Compound 29

Accordingly, compound 29 was prepared in the same way as compound 7 starting from intermediate AC-2 (0.54 mmol) and intermediate A-5 (0.35 mmol) yielding 0.12 g (66%) as a white powder.

¹H NMR (400 MHz, DMSO) δ 9.21 (s, 1H), 8.51 (d, J=1.8 Hz, 1H), 8.46 (t, J=5.7 Hz, 1H), 8.00-7.94 (m, 2H), 7.48 (d, J=7.3 Hz, 2H), 4.96 (s, 2H), 4.59 (d, J=5.7 Hz, 2H), 4.37 (t, J=5.2 Hz, 2H), 4.16 (d, J=4.9 Hz, 2H), 2.64 (d, J=1.3 Hz, 3H), 2.34 (s, 3H).

### Synthesis of Compound 30

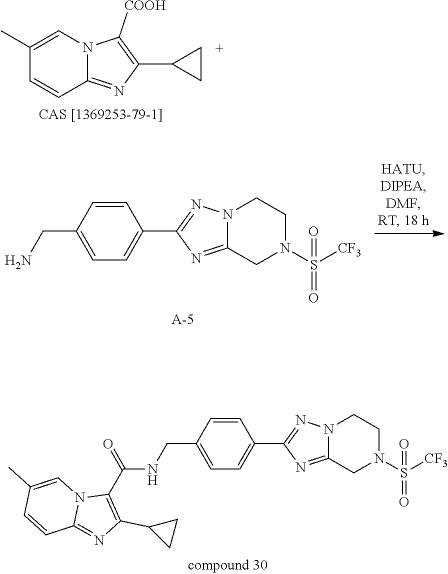 [SMILES: Cc1ccc2nc(C3CC3)c(C(=O)O)n2c1 ; NCc1ccc(-c2nc3n(n2)CCN(S(=O)(=O)C(F)(F)F)C3)cc1 ; "A-5" ; Cc1ccc2nc(C3CC3)c(C(=O)NCc3ccc(-c4nc5n(n4)CCN(S(=O)(=O)C(F)(F)F)C5)cc3)n2c1]

Accordingly, compound 30 was prepared in the same way as compound 7 starting from 2-cyclopropyl-6-methylimidazo[1,2-a]pyridine-3-carboxylic acid CAS [1369253-79-1] (0.52 mmol) and intermediate A-5 (0.35 mmol) yielding 0.13 g (65%) as a white powder.

¹H NMR (400 MHz, DMSO) δ 8.84 (s, 1H), 8.52 (t, J=6.0 Hz, 1H), 7.97 (d, J=8.2 Hz, 2H), 7.47 (dd, J=11.9, 8.7 Hz, 3H), 7.23 (dd, J=9.1, 1.5 Hz, 1H), 4.96 (s, 2H), 4.60 (d, J=5.9 Hz, 2H), 4.36 (t, J=5.4 Hz, 2H), 4.15 (t, J=5.1 Hz, 2H), 2.45-2.37 (m, 1H), 2.30 (s, 3H), 1.00 (d, J=6.0 Hz, 4H).

### Synthesis of Compound 31

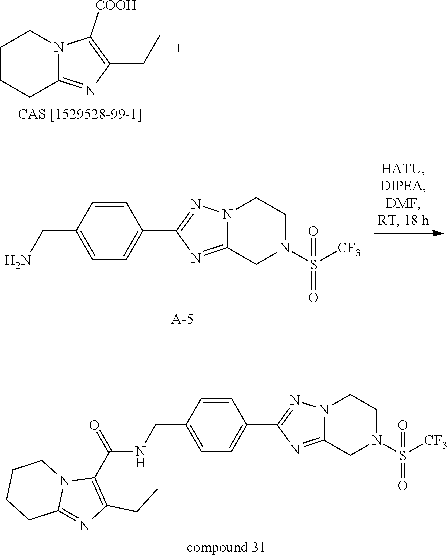 [SMILES: CCc1nc2n(c1C(=O)O)CCCC2 ; NCc1ccc(-c2nc3n(n2)CCN(S(=O)(=O)C(F)(F)F)C3)cc1 ; "A-5" ; CCc1nc2n(c1C(=O)NCc1ccc(-c3nc4n(n3)CCN(S(=O)(=O)C(F)(F)F)C4)cc1)CCCC2]

Accordingly, compound 31 was prepared in the same way as compound 7 starting from 2-ethyl-5H,6H,7H,8H-imidazo[1,2-a]pyridine-3-carboxylic acid CAS [1529528-99-1] (0.41 mmol) and intermediate A-5 (0.33 mmol) yielding 0.1 g (57%) as a white solid.

¹H NMR (400 MHz, DMSO) δ 8.27 (t, J=6.0 Hz, 1H), 7.96 (d, J=8.2 Hz, 2H), 7.41 (d, J=8.2 Hz, 2H), 4.96 (s, 2H), 4.47 (d, J=6.0 Hz, 2H), 4.41-4.33 (m, 2H), 4.22-4.14 (m, 2H), 4.04-3.95 (m, 2H), 2.74-2.69 (m, 2H), 2.65 (q, J=7.5 Hz, 2H), 1.90-1.83 (m, 2H), 1.83-1.74 (m, 2H), 1.11 (t, J=7.5 Hz, 3H).

### Synthesis of Compound 33

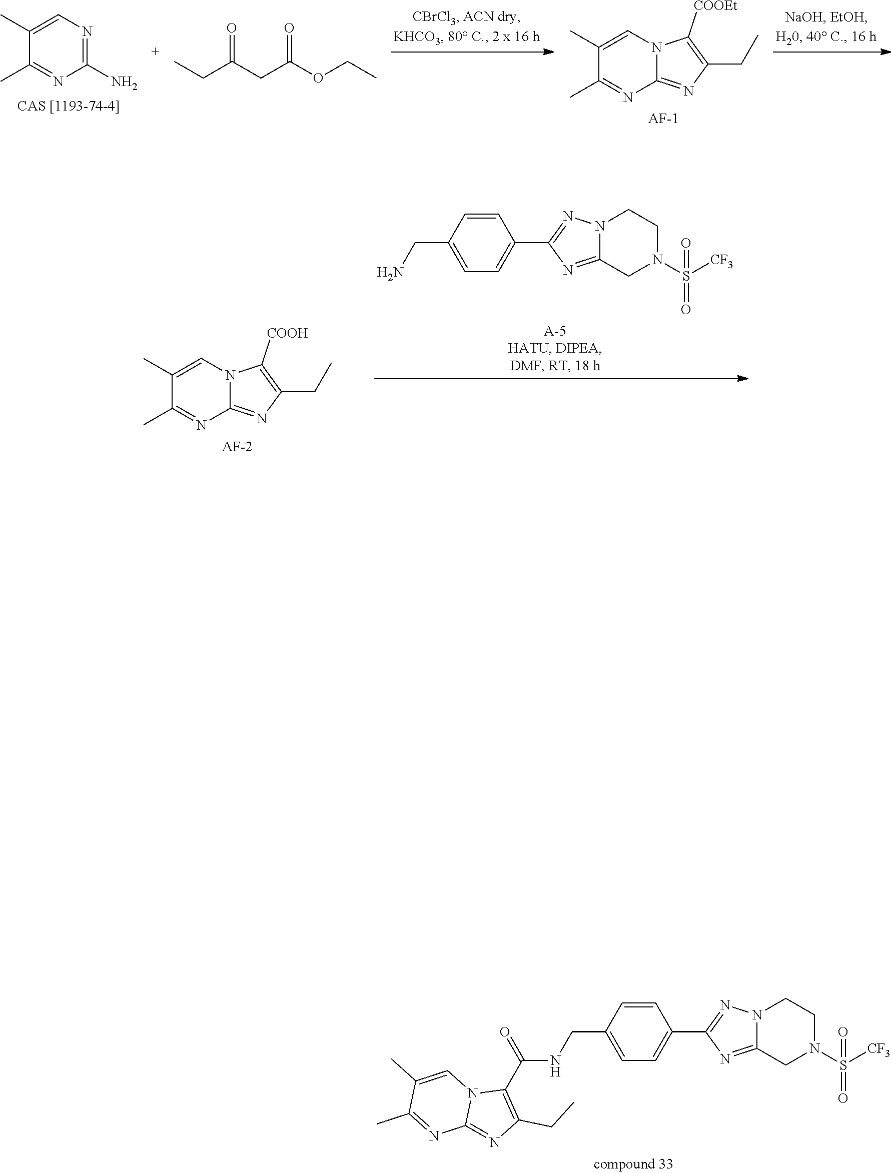

### Preparation of Compound AF-1

Potassium bicarbonate (1 eq) and Ethyl acetoacetate (1.5 eq) were added to a solution of 4,5-dimethyl-2-pyrimidinamine (1 eq, limiting reagent) in Acetonitrile dry (40 eq) in a screw top vial at rt. Then, Bromotricloromethane (3 eq) was added at rt and the mixture was stirred at 80° C. for 16 h. LCMS analysis showed desired product and starting material. Additional load of Ethyl acetoacetate (0.5 eq), and Bromotricloromethane (1 eq) were added and the reaction mixture stirred at 80° C. for additional 16 h. Saturated aqueous NaHCO₃ solution was added and the mixture was extracted with EtOAc (×3). The combined organic layers were dried over MgSO4, filtered and concentrated in vacuo. The crude product was purified by flash column chromatography (SiOH, 12 g; DCM/MeOH 9:1 in DCM 0/100 to 20/80). The desired fractions were collected and concentrated in vacuo to yield intermediate AF-1 as a brown solid (Yield: 26%).

### Preparation of Intermediate AF-2

Accordingly, intermediate AF-2 was prepared in the same way as intermediate AL-2 starting from intermediate AF-1 (0.61 mmol) giving 0.13 g (86%).

### Preparation of Compound 33

Accordingly, compound 33 was prepared in the same way as compound 7 starting from intermediate AF-2 (0.52 mmol) and intermediate A-5 (0.35 mmol) yielding 0.12 g (61%) as a white solid.

¹H NMR (400 MHz, DMSO) δ 9.06 (s, 1H), 8.42 (t, J=6.0 Hz, 1H), 7.96 (d, J=8.2 Hz, 2H), 7.46 (d, J=8.2 Hz, 2H), 4.95 (s, 2H), 4.57 (d, J=5.9 Hz, 2H), 4.36 (t, J=5.4 Hz, 2H), 4.15 (t, J=5.3 Hz, 2H), 2.99 (q, J=7.5 Hz, 2H), 2.27 (s, 3H), 1.26 (t, J=7.5 Hz, 3H). —CH3 was overlapped with DMSO peak.

### Synthesis of Compound 34

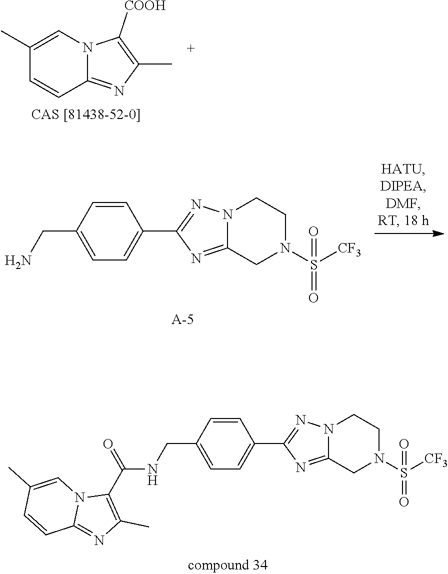

Accordingly, compound 34 was prepared in the same way as compound 7 starting from 2,6-Dimethylimidazo[1,2-a]pyridine-3-carboxylic acid CAS [81438-52-0] (0.43 mmol) and intermediate A-5 (0.33 mmol) yielding 0.095 g (54%) as a white solid.

¹H NMR (400 MHz, DMSO-d6) δ 8.87 (s, 1H), 8.35 (t, J=6.0 Hz, 1H), 8.01-7.94 (m, 2H), 7.48 (dd, J=8.6, 4.6 Hz, 3H), 7.25 (dd, J=9.1, 1.6 Hz, 1H), 4.96 (s, 2H), 4.58 (d, J=5.9 Hz, 2H), 4.36 (t, J=5.4 Hz, 2H), 4.15 (t, J=5.1 Hz, 2H), 2.59 (s, 3H), 2.31 (s, 3H).

### Synthesis of Compound 42

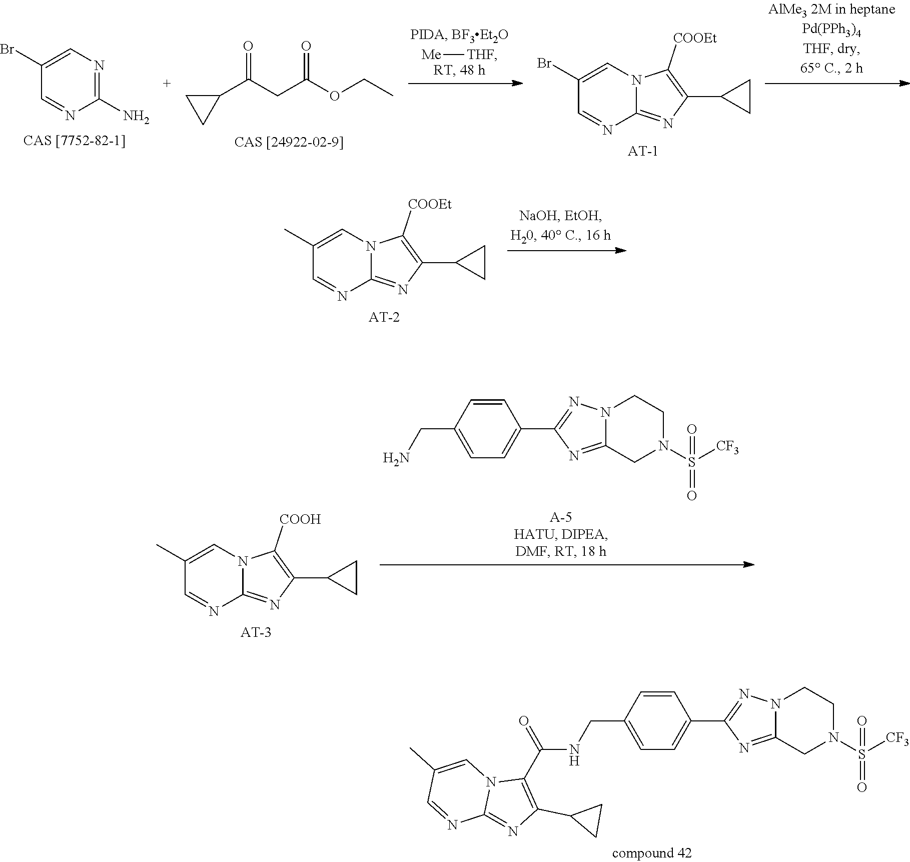

### Preparation of Intermediate AT-1

In a screw top vial, Boron trifluoride diethyl etherate (0.071 mL, 0.57 mmol) was added dropwise to a solution of 2-Amino-5-bromopyrimidine (CAS [7752-82-1], 1 g, 5.75 mmol), Ethyl-3-cyclopropyl-3-oxopropionate (1.27 mL, 8.62 mmol) and (Diacetoxyiodo)benzene (2.8 g, 8.62 mmol) in 2-MeTHF dry (25 mL) under nitrogen at rt and the mixture was stirred at 60° C. for 16 h. Water was added and the mixture was extracted with EtOAc. The layers were separated, and the organic layer was washed with saturated aqueous NaHCO₃ solution and brine. The combined organic layer was dried over MgSO₄, filtered and concentrated in vacuo. The crude product was purified by flash column chromatography (SiOH, 80 g; Ethyl acetate/Heptane from 0/100 to 25/75). The desired fractions were collected and concentrated in vacuo to yield intermediate AT-1 (0.66 g, 37%) as a yellow solid.

### Preparation of Intermediate AT-2

Accordingly, intermediate AT-2 was prepared in the same way as intermediate AC-1 starting from intermediate AT-1 (3.64 mmol) giving 0.73 g (81%).

### Preparation of Intermediate AT-3

Accordingly, intermediate AT-3 was prepared in the same way as intermediate AL-2 starting from intermediate AT-2 (0.61 mmol) giving 0.13 g (99%).

### Preparation of Compound 42

Accordingly, compound 42 was prepared in the same way as compound 7 starting from intermediate AT-3 (0.48 mmol) and intermediate A-5 (0.35 mmol) yielding 0.12 g (61%) as a white solid.

¹H NMR (400 MHz, DMSO) δ 9.18 (s, 1H), 8.64 (t, J=5.8 Hz, 1H), 8.49 (d, J=2.5 Hz, 1H), 7.97 (d, J=8.3 Hz, 2H), 7.48 (d, J=8.3 Hz, 2H), 4.96 (s, 2H), 4.60 (d, J=5.7 Hz, 2H), 4.36 (t, J=5.3 Hz, 2H), 4.16 (d, J=5.1 Hz, 2H), 2.67 (m, 1H), 2.33 (s, 2H), 1.06 (d, J=6.0 Hz, 4H).

### Synthesis of Compound 44

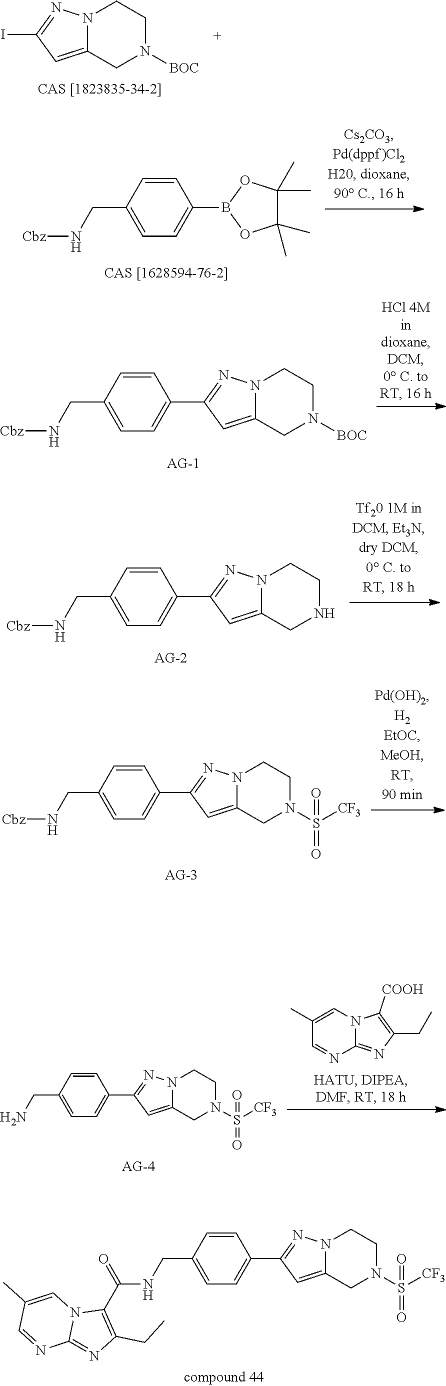

### Preparation of Intermediate AG-1

Accordingly, intermediate AG-1 was prepared in the same way as intermediate AE-1 starting from pyrazine-5(4H)-carboxylate (CAS [1823835-34-2], 0.73 mmol) and benzyl 4-(4,4,5,5-tetramethyl-1,3,2-dioxaborolan-2-yl)benzylcarbamate (CAS [1628594-76-2], 0.88 mmol) affording 0.13 g (35%) as white solid.

### Preparation of Intermediate AG-2

Accordingly, intermediate AG-2 was prepared in the same way as intermediate AE-6 starting from AG-1 (0.27 mmol) yielding 0.11 g (100%) as an orange powder.

### Preparation of Intermediate AG-3

Accordingly, intermediate AG-3 was prepared in the same way as intermediate A-3 starting from AG-2 (0.27 mmol) yielding 0.06 g (45%) as white powder.

### Preparation of Intermediate AG-4

Accordingly, intermediate AG-4 was prepared in the same way as intermediate AE-2 starting from AG-3 (0.13 mmol) yielding 0.045 g (95%) as white solid.

### Preparation of Compound 44

Accordingly, compound 44 was prepared in the same way as compound 7 starting from intermediate AG-4 (0.23 mmol) and intermediate A-5 (0.14 mmol) yielding 0.027 g (34%) as a white powder.

¹H NMR (400 MHz, DMSO) δ 9.17-9.13 (m, 1H), 8.52-8.46 (m, 2H), 7.75 (d, J=8.2 Hz, 2H), 7.41 (d, J=8.3 Hz, 2H), 6.63 (s, 1H), 4.87 (s, 2H), 4.55 (d, J=5.9 Hz, 2H), 4.28 (t, J=5.5 Hz, 2H), 4.10 (t, J=5.4 Hz, 2H), 3.02 (q, J=7.5 Hz, 2H), 2.34 (s, 3H), 1.28 (t, J=7.5 Hz, 3H).

### Synthesis of Compound 45

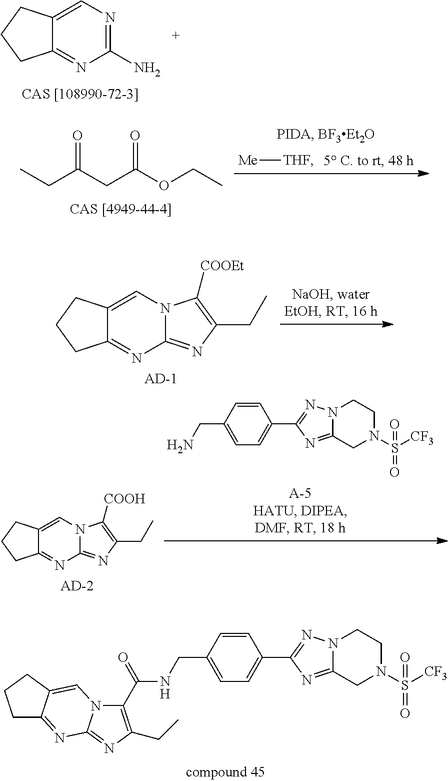

### Preparation of Intermediate AD-1

Accordingly, compound AD-1 was prepared in the same way as compound AL-1 starting from 6,7-dihydro-5h-cyclopenta[d]pyrimidin-2-amine (CAS [108990-72-3], 7.4 mmol) affording 0.726 g (38%).

### Preparation of Intermediate AD-2

Accordingly, compound AD-2 was prepared in the same way as compound AL-2 starting from AD-1 (0.77 mmol) affording 0.446 g (44%).

### Preparation of Compound 45

Accordingly, compound 45 was prepared in the same way as compound 7 starting from intermediate AD-2 (0.60 mmol) and intermediate A-5 (0.38 mmol) affording 0.036 g (16%) as white powder.

¹H NMR (400 MHz, DMSO) δ 9.11 (s, 1H), 8.45 (t, J=6.0 Hz, 1H), 7.97 (d, J=8.3 Hz, 2H), 7.47 (d, J=8.3 Hz, 2H), 4.96 (s, 2H), 4.57 (d, J=5.9 Hz, 2H), 4.36 (t, J=5.4 Hz, 2H), 4.15 (t, J=5.3 Hz, 2H), 3.04-2.90 (m, 6H), 2.13 (p, J=7.6 Hz, 2H), 1.26 (t, J=7.5 Hz, 3H).

### Synthesis of Compounds 47, 48 and 51

### Preparation of Intermediate AI-3

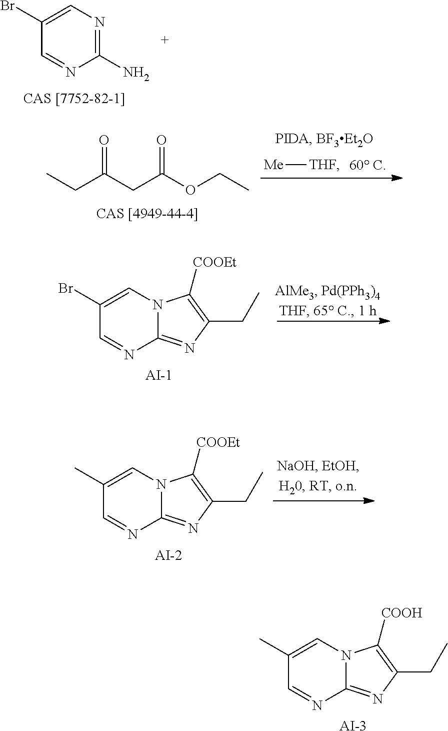

### Preparation of Intermediate AI-1

2-amino-5-bromopyrimidine (10.0 g; 57.5 mmol) was suspended in dry 2-MeTHF (250 mL). ethyl 3-oxovalerate (8.2 mL, 57.5 mmol, 1 eq.) and iodobenzene diacetate (18.5 g, 57.5 mmol, 1 eq.) were added. boron trifluoride etherate (0.75 mL, 2.87 mmol, 0.05 eq.) was then added dropwise and the reaction mixture was stirred at 60° C. for 1.5 hours. An extra amount of ethyl ethyl 3-oxovalerate (4.10 mL, 28.7 mmol, 0.5 eq.), iodobenzene diacetate (9.25 g, 28.7 mmol, 0.5 eq.) and boron trifluoride etherate (0.75 mL, 2.87 mmol, 0.05 eq.) were added at room temperature and the mixture was stirred at 60° C. for 1h. The mixture was cooled down to room temperature then EtOAc and water were added. The organic layer was separated and washed with a saturated solution of NaHCO₃ (twice), then with brine (twice). The organic layer was dried over MgSO₄, filtered off and concentrated to give 19.7 g as a brown oil. The crude was purified by preparative LC (irregular SiOH, 15-40 μm, 330 g, dry loading (SiOH), mobile phase gradient: from DCM 100% to DCM 85%, EtOAc 15%) to give intermediate AI-1, 9.03 g as yellow crystals (53%).

### Preparation of Intermediate AI-2

In a sealed tube under N₂, to a solution of intermediate AI-1 (500 mg, 1.68 mmol) and Pd(PPh₃)₄ (96.9 mg, 0.084 mmol) in THF (12 mL) degassed under N2 was added trimethylaluminum 2 m in Hexanes (2 eq., 1.68 mL, 3.35 mmol). The mixture was purged again with N2 and was heated at 65° C. for 1 h. An extra amount of trimethylaluminum 2 m in Hexanes (1 eq., 0.839 mL, 1.68 mmol) was added and the mixture was stirred at 65° C. for 1 h. The mixture was diluted with DCM, cooled down to 0° C. and 1 mL of water was added carefully. The mixture was stirred at room temperature overnight then MgSO₄ was added. After 30 min under stirring, the mixture was filtered over a plug of Celite® and evaporated to give 412 mg of as an orange gum. The crude was purified by preparative LC (regular SiOH, 30 μm, 40 g, dry loading (Celite®), mobile phase eluent: Heptane 95%, EtOAc 5% to Heptane 50%, EtOAc 50%). Fractions containing product were combined and concentrated to obtain intermediate AI-2, 354 mg of as a yellow gum (90%).

### Preparation of Intermediate AI-3

To a solution of intermediate AI-2 (120 mg, 0.514 mmol) in water (1 mL) and EtOH (4 mL) was added NaOH (62 mg, 1.55 mmol) and the mixture was stirred at room temperature overnight. The mixture was evaporated then co-evaporated with EtOH to give intermediate AI-3, 190 mg as a yellow solid. The crude was used as such in next step.

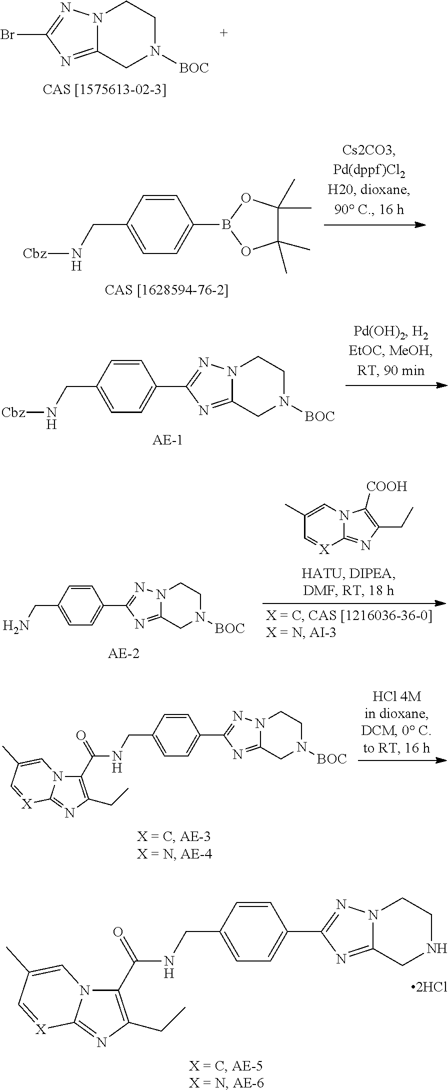

### Preparation of Intermediate AE-1

In a glass pressure bottle, a stirred mixture of tert-butyl 2-bromo-5,6-dihydro[1,2,4]triazolo[1,5-a]pyrazine-7(8H)-carboxylate (CAS [1575613-02-3], 1.02 g, 3.37 mmol), benzyl 4-(4,4,5,5-tetramethyl-1,3,2-dioxaborolan-2-yl)benzylcarbamate (CAS [1628594-76-2], 1.73 g, 4.72 mmol) and [1,1′-Bis(diphenylphosphino)ferrocene]dichloropalladium(II), complex with dichloromethane (0.28 g, 0.34 mmol) in dioxane (16 mL) and water (8 mL) while was bubbled with nitrogen. Then Cs₂CO₃ (2.2 g, 6.75 mmol) was added at room temperature. The mixture was stirred at 90° C. for 16 h. The reaction was cooled, diluted with water and extracted with EtOAc (×3). The combined organic layers were dried over MgSO₄, filtered and concentrated in vacuo. The crude product was purified by flash column chromatography (SiOH, 12 g; EtOAc in Heptane (0/100 to 60/40)). The desired fractions were collected and concentrated in vacuo to yield intermediate AE-1 as a beige solid (1.34 g, 85%).

### Preparation of Intermediate AE-2

Palladium hydroxide on carbon (0.2 g, 0.29 mmol) was added to a stirred solution of AE-1 (1.34 g, 2.89 mmol) in EtOAc (10 mL), and MeOH (3 mL) at room temperature under nitrogen atmosphere. Then, nitrogen atmosphere was replaced by H₂ (P atm) and the reaction mixture was stirred at room temperature for 1.5 h. The mixture was filtered through of pad of Celite®, and solvents was concentrated in vacuo to yield AE-2 as a white solid (0.85 g, 84%).

### Preparation of Intermediate AE-4

Intermediate AE-2 (0.85 g, 2.57 mmol) was added to a solution of AI-3 (1.08 g, 4.11 mmol), HATU (1.46 g, 3.85 mmol) and DIPEA (3.13 mL, 13.98 mmol) in DMF dry in a round bottom flask at room temperature. The mixture was stirred at room temperature for 16 h. Saturated aqueous NaHCO₃ solution was added and the mixture was extracted with EtOAc (×3). The combined organic layers were dried over MgSO₄, filtered and concentrated in vacuo. The crude product was purified by flash column chromatography (SiOH, 25 g; DCM/MeOH 9:1 in DCM 0/100 to 20/80). The desired fractions were collected and concentrated in vacuo to yield AE-4 (1.32 g, 95%) as a clear brown solid.

### Preparation of Intermediate AE-3

Accordingly, intermediate AE-3 is prepared in the same way as intermediate AE-4 starting from 2-ethyl-6-methylimidazo[1,2-a]pyridine-3-carboxylic acid (CAS [1216036-36-0]).

### Preparation of Intermediate AE-6

HCl in dioxane (4M) (3.83 mL, 15.33 mmol) was added to a stirred solution of AE-4 and DCM (20 mL) in a round bottom flask at room temperature. The mixture was stirred at room temperature for 16 h. Solvents were removed in vacuo and the solid was triturated with DIPE to yield AE-6 (1.25 g, qtve) as a beige solid (HCl salt). The crude product was used as such in the next step.

### Preparation of Intermediate AE-5

Accordingly, intermediate AE-5 is prepared in the same way as intermediate AE-6 starting from intermediate AE-3.

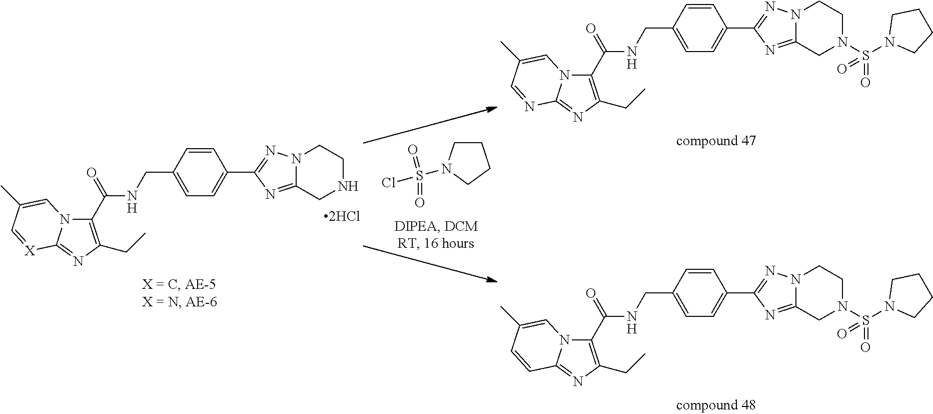

### Preparation of Compound 47

Pyrrolidine-1-sulfonyl chloride (0.046 mL, 0.27 mmol) was added to a solution of AE-6 (0.12 g, 0.25 mmol) and DIPEA (0.085 mL, 0.49 mmol) in DCM dry (5 mL) in a round bottom flask under nitrogen at room temperature. The mixture was stirred at room temperature for 16 h. Saturated aqueous NaHCO₃ solution was added and extracted with DCM. The organic layer was separated, dried (MgSO₄), filtered and the solvents evaporated in vacuo. The crude product was purified by flash column chromatography (SiOH, 12 g; (DCM/MeOH (9:1)) in DCM from 0/100 to 100/0). The desired fractions were collected and concentrated in vacuo. The result was triturated with DIPE and the solid was filtrated to yield 0.057 g of compound 47 as a pale beige solid (42%).

¹H NMR (400 MHz, DMSO) δ 9.16 (d, J=1.2 Hz, 1H), 8.50 (dd, J=7.7, 4.2 Hz, 2H), 7.96 (d, J=8.2 Hz, 2H), 7.46 (d, J=8.2 Hz, 2H), 4.63-4.49 (m, 4H), 4.27 (t, J=5.4 Hz, 2H), 3.80 (t, J=5.4 Hz, 2H), 3.28 (t, J=6.7 Hz, 4H), 3.02 (q, J=7.5 Hz, 2H), 2.34 (s, 3H), 1.93-1.79 (m, 4H), 1.33-1.23 (m, 3H).

### Preparation of Compound 48

Accordingly, compound 48 was prepared in the same way as compound 47 starting from AE-5 (0.33 mmol) affording 0.12 g (64%).

¹H NMR (400 MHz, DMSO) δ 8.81 (s, 1H), 8.38 (t, J=5.9 Hz, 1H), 7.96 (d, J=8.2 Hz, 2H), 7.51 (d, J=9.1 Hz, 1H), 7.46 (d, J=8.2 Hz, 2H), 7.24 (dd, J=9.1, 1.5 Hz, 1H), 4.57 (d, J=7.2 Hz, 4H), 4.27 (t, J=5.4 Hz, 2H), 3.80 (t, J=5.4 Hz, 2H), 3.27 (d, J=6.7 Hz, 4H), 2.98 (q, J=7.5 Hz, 2H), 2.31 (s, 3H), 1.94-1.80 (m, 4H), 1.26 (t, J=7.5 Hz, 3H).

### Preparation of compound 51

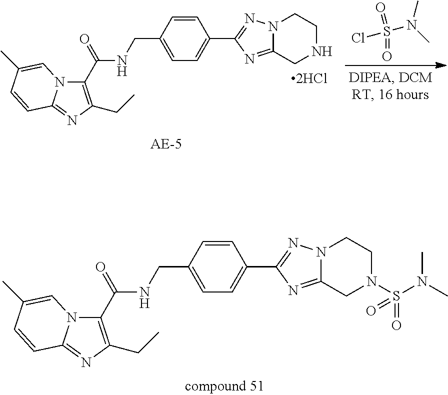

Accordingly, compound 51 was prepared in the same way as compound 47 starting from AE-5 (0.33 mmol) and N,N-Dimethylsulfamoyl chloride affording (0.69 mmol) yielding 0.07 g (40%).

¹H NMR (400 MHz, DMSO) δ 8.81 (s, 1H), 8.40 (t, J=6.0 Hz, 1H), 7.96 (d, J=8.2 Hz, 2H), 7.51 (d, J=9.1 Hz, 1H), 7.46 (d, J=8.2 Hz, 2H), 7.24 (dd, J=9.1, 1.6 Hz, 1H), 4.60-4.55 (m, 4H), 4.27 (t, J=5.4 Hz, 2H), 3.81 (t, J=5.4 Hz, 2H), 2.98 (q, J=7.5 Hz, 2H), 2.83 (s, 6H), 2.31 (s, 3H), 1.26 (t, J=7.5 Hz, 3H).

### Synthesis of Compound 55

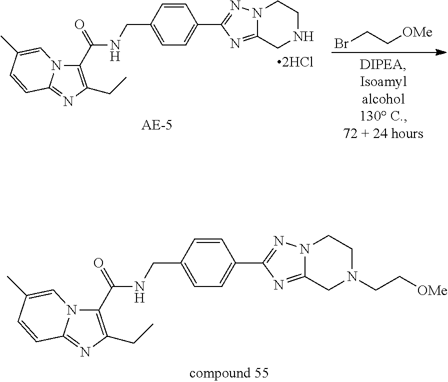 [SMILES: CCc1nc2ccc(C)cn2c1C(=O)NCc1ccc(-c2nc3n(n2)CCNC3)cc1 ; COCCBr ; CCc1nc2ccc(C)cn2c1C(=O)NCc1ccc(-c2nc3n(n2)CCN(CCOC)C3)cc1]

To a solution of AE-5 (0.15 g, 0.31 mmol) in Isoamyl alcohol (2 mL) was added DIPEA (0.16 g, 1.23 mmol) and 2-Bromoethyl methyl ether (0.032 mL, 0.34 mmol) at room temperature, the mixture was stirred at 130° C. for 72 hours. Reaction mixture was recharged with DIPEA (1.5 eq.) and Isoamyl alcohol (0.5 mL) and stirred at 130° C. for 24 h. The mixture was diluted with DCM and washed with saturated aqueous NaHCO₃ solution. The organic layer was dried over MgSO₄, filtered and concentrated under reduced pressure. The crude was purified by flash column chromatography (SiOH; DCM/MeOH (9/1) in DCM from 0/100 to 30/70). The desired fractions were collected and concentrated under vacuum. The product was triturated with DIPE to yield 0.037 g of compound 55 (25%) as a white solid.

¹H NMR (400 MHz, DMSO) δ 8.80 (s, 1H), 8.40 (t, J=6.0 Hz, 1H), 7.95 (d, J=8.2 Hz, 2H), 7.51 (d, J=9.2 Hz, 1H), 7.44 (d, J=8.3 Hz, 2H), 7.25 (dd, J=9.1, 1.7 Hz, 1H), 4.56 (d, J=5.9 Hz, 2H), 4.16 (t, J=5.4 Hz, 2H), 3.82 (s, 2H), 3.54 (t, J=5.5 Hz, 2H), 3.27 (s, 3H), 3.05 (t, J=5.5 Hz, 2H), 2.98 (q, J=7.5 Hz, 2H), 2.78 (t, J=5.5 Hz, 2H), 2.31 (s, 3H), 1.26 (t, J=7.5 Hz, 3H)

### Synthesis of Compound 56

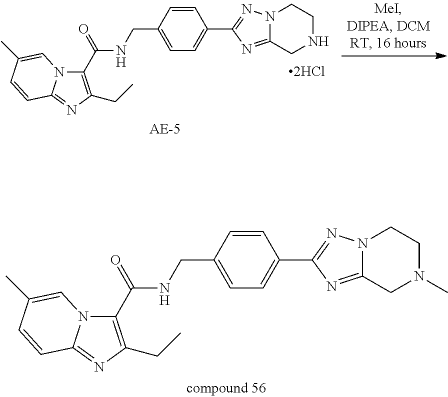

Accordingly, compound 56 was prepared in the same way as compound 47 starting from AE-5 (0.31 mmol) and iodomethane (0.46 mmol) yielding 0.047 g (35%).

¹H NMR (400 MHz, DMSO) δ 8.80 (s, 1H), 8.40 (t, J=5.6 Hz, 1H), 7.95 (d, J=8.0 Hz, 2H), 7.51 (d, J=9.1 Hz, 1H), 7.44 (d, J=8.0 Hz, 2H), 7.24 (d, J=9.0 Hz, 1H), 4.56 (d, J=5.6 Hz, 2H), 4.17 (t, J=5.1 Hz, 2H), 3.70 (s, 2H), 2.98 (q, J=7.5 Hz, 2H), 2.92 (t, J=5.3 Hz, 2H), 2.44 (s, 3H), 2.31 (s, 3H), 1.26 (t, J=7.4 Hz, 3H).

### Synthesis of Compound 65

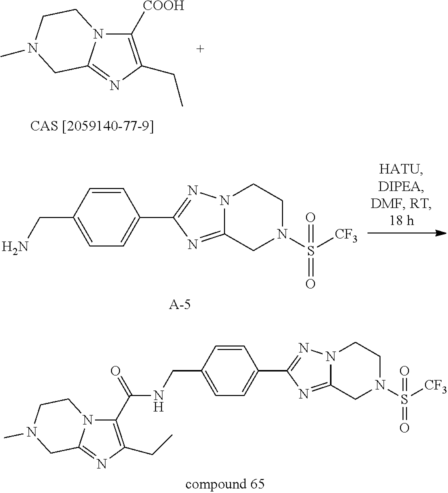 [SMILES: CCc1nc2n(c1C(=O)O)CCN(C)C2 ; NCc1ccc(-c2nc3n(n2)CCN(S(=O)(=O)C(F)(F)F)C3)cc1 ; "A-5" ; CCc1nc2n(c1C(=O)NCc1ccc(-c3nc4n(n3)CCN(S(=O)(=O)C(F)(F)F)C4)cc1)CCN(C)C2]

Accordingly, compound 65 was prepared in the same way as compound 1 starting from 2-ethyl-7-methyl-6,8-dihydro-5H-imidazo[1,2-a]pyrazine-3-carboxylic acid (CAS [2059140-77-9], 0.66 mmol) and intermediate A-5 (0.44 mmol) affording 0.051 g (20%) as white solid.

1H NMR (400 MHz, DMSO-d6, 100° C.) δ 7.96 (d, J=8.0 Hz, 2H), 7.87 (t, J=5.3 Hz, 1H), 7.42 (d, J=8.0 Hz, 2H), 4.93 (s, 2H), 4.50 (d, J=5.9 Hz, 2H), 4.37 (t, J=5.5 Hz, 2H), 4.16 (t, J=5.4 Hz, 2H), 4.07 (t, J=5.5 Hz, 2H), 3.53 (s, 2H), 2.76 (t, J=5.5 Hz, 2H), 2.70 (q, J=7.4 Hz, 2H), 2.39 (s, 3H), 1.15 (t, J=7.5 Hz, 3H).

### Synthesis of Compound 66

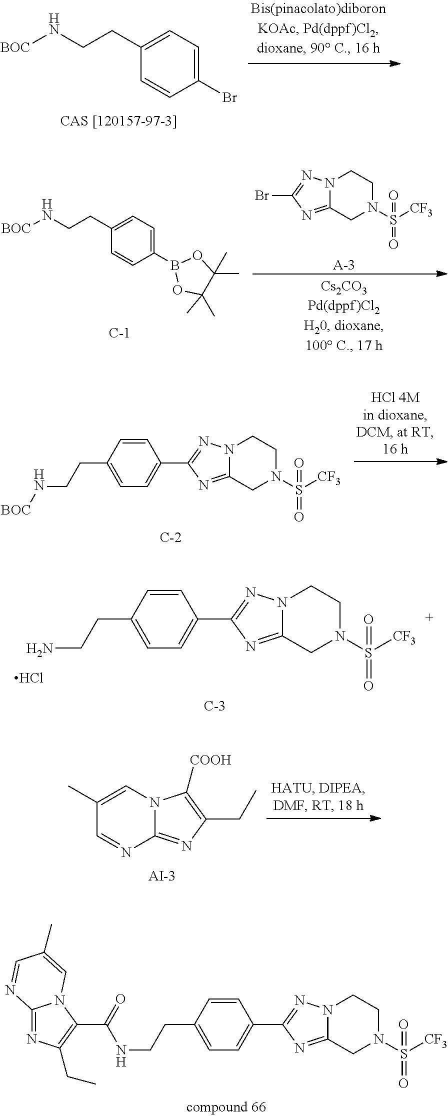

### Preparation of Intermediate C-1

To a N₂ purged solution of tert-butyl N-[2-(4-bromophenyl)ethyl]carbamate (CAS [120157-97-3] 0.9 g, 3 mmol), Bis(pinacolato)diboron (1.14 g, 4.5 mmol) and Potassium acetate (0.88 g, 8.9 mmol) in 1,4-dioxane (24 mL) was added [1,1′-Bis(diphenylphosphino)ferrocene] dichloropalladium(II) (245 mg, 0.3 mmol), then the reaction mixture was stirred at 90° C. for 18 h. The reaction mixture was filtered on Celite®, the filter cake was rinsed with EtOAc and the filtrate was concentrated under reduced pressure. The residue was purified by flash chromatography over silica gel (MeOH in DCM 0/100 to 4/96) to afford intermediate C-1 as a beige solid, 0.7 g (64%).

### Preparation of Intermediate C-2

A N₂ purged mixture of intermediate C-1 (250 mg, 0.72 mmol), intermediate A-3 (219 mg, 0.65 mmol), Cesium carbonate (469 mg, 1.44 mmol) and 1,1′-Bis(diphenylphosphino) ferrocene dichloropalladium (II) (80 mg, 0.098 mmol) in 1,4-dioxane (4 mL) and water (1.8 mL) was stirred at 100° C. for 17 h. The reaction mixture was cooled to rt, filtered on Celite® and the filter cake was washed with EtOAc. The filtrate was concentrated and purified by flash chromatography over silica gel (EtOAc in Heptane from 0/100 to 25/75) to afford intermediate C-2 as a white solid, 0.256 g (81%).

### Preparation of Intermediate C-3

To a nitrogen-purged mixture of C-2 (256 mg, 0.54 mmol) in dry DCM (10 mL) was added 4M solution of HCl in 1,4-dioxane (0.81 mL, 3.23 mmol) at RT. The reaction mixture was stirred for 16 h. The reaction mixture was concentrated under reduced pressure to afford intermediate C-3 as a white solid, 0.216 g (89%).

### Preparation of Compound 66

Accordingly, compound 66 was prepared in the same way as compound 1 starting from intermediate AI-3 2-ethyl-6-methyl-imidazo[1,2-a]pyrimidine-3-carboxylic acid (0.58 mmol) and intermediate C-3 (0.48 mmol) yielding 0.171 g (61%) as a white powder. 1H NMR (400 MHz, DMSO) δ 8.99 (s, 1H), 8.48 (d, J=2.2 Hz, 1H), 8.01 (t, J=5.5 Hz, 1H), 7.93 (d, J=8.0 Hz, 2H), 7.38 (d, J=8.1 Hz, 2H), 4.96 (s, 2H), 4.36 (t, J=5.3 Hz, 2H), 4.22-4.08 (m, 2H), 3.61 (dd, J=12.8, 6.6 Hz, 2H), 2.94 (t, J=7.1 Hz, 2H), 2.83 (q, J=7.5 Hz, 2H), 2.29 (s, 3H), 1.17 (t, J=7.5 Hz, 3H).

### Synthesis of Compound 67

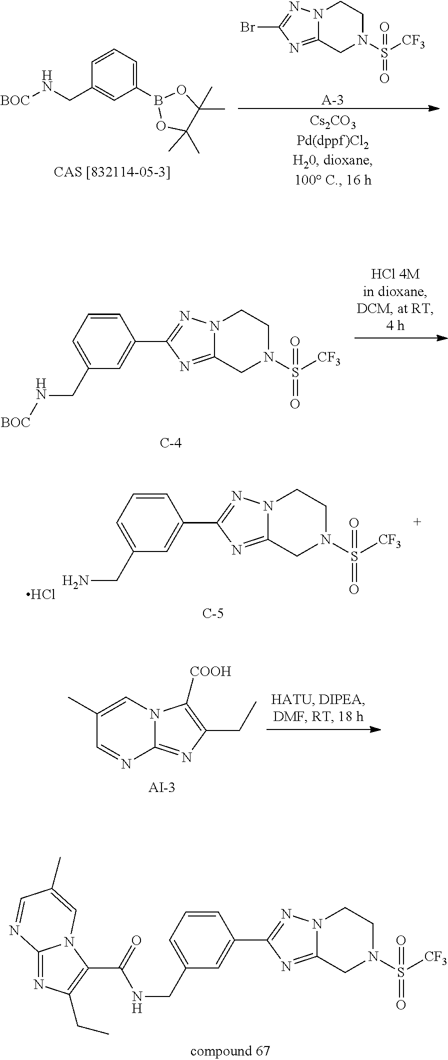

### Preparation of Intermediate C-4

Accordingly, intermediate C-4 was prepared in the same way as intermediate C-2 starting from tert-butyl N-[[3-(4,4,5,5-tetramethyl-1,3,2-dioxaborolan-2-yl)phenyl]methyl]carbamate (CAS [832114-05-3], 273 mg, 0.82 mmol), intermediate A-3 (250 mg, 0.75 mmol), affording intermediate C-4 as a white solid, 0.169 g (47%).

### Preparation of Intermediate C-5

Accordingly, intermediate C-5 was prepared in the same way as intermediate C-3 starting from intermediate C-4 (165 mg, 0.36 mmol) to afford intermediate C-5 as a white solid, 0.154 g (98%).

### Preparation of Compound 67

Accordingly, compound 67 was prepared in the same way as compound 1 starting from intermediate AI-3 2-ethyl-6-methyl-imidazo[1,2-a]pyrimidine-3-carboxylic acid (0.49 mmol) and intermediate C-5 (0.35 mmol) yielding 0.067 g (34%) as a white powder.

1H NMR (400 MHz, DMSO) δ 9.14 (s, 1H), 8.58 (t, J=5.8 Hz, 1H), 8.52 (s, 1H), 8.05 (s, 1H), 7.88 (d, J=4.1 Hz, 1H), 7.51-7.40 (m, 2H), 4.95 (s, 2H), 4.60 (d, J=5.8 Hz, 2H), 4.36 (t, J=5.1 Hz, 2H), 4.15 (s, 2H), 3.03 (q, J=7.5 Hz, 2H), 2.34 (s, 3H), 1.28 (t, J=7.4 Hz, 3H)

### Synthesis of Compound 68

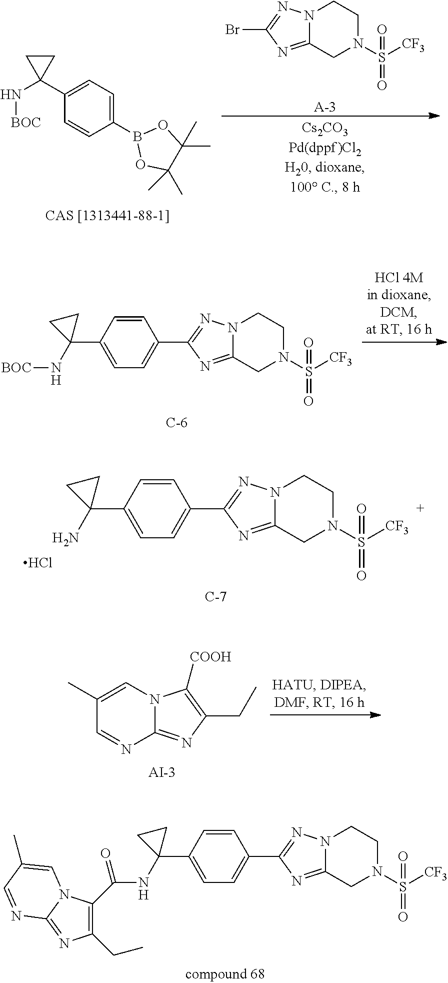

### Preparation of Intermediate C-6

Accordingly, intermediate C-6 was prepared in the same way as intermediate C-2 starting from tert-butyl N-[1-[4-(4,4,5,5-tetramethyl-1,3,2-dioxaborolan-2-yl)phenyl]cyclopropyl]carbamate (CAS [1313441-88-1], 483 mg, 1.35 mmol), intermediate A-3 (410 mg, 1.22 mmol), affording intermediate C-6 as a white solid, 0.337 g (56%).

### Preparation of Intermediate C-7

Accordingly, intermediate C-7 was prepared in the same way as intermediate C-3 starting from intermediate C-6 (322 mg, 0.66 mmol) to afford intermediate C-7 as a white solid, 0.331 g (99%).

### Preparation of Compound 68

Accordingly, compound 68 was prepared in the same way as compound 1 starting from intermediate AI-3 2-ethyl-6-methyl-imidazo[1,2-a]pyrimidine-3-carboxylic acid (0.41 mmol) and intermediate C-7 (0.32 mmol) yielding 0.170 g (92%) as a white powder.

1H NMR (400 MHz, DMSO) δ 9.05 (dd, J=2.3, 1.1 Hz, 1H), 8.76 (s, 1H), 8.51 (d, J=2.4 Hz, 1H), 7.91 (d, J=8.5 Hz, 2H), 7.34 (d, J=8.5 Hz, 2H), 4.95 (s, 2H), 4.36 (t, J=5.3 Hz, 2H), 4.20-4.11 (m, 2H), 3.03 (q, J=7.5 Hz, 2H), 2.33 (s, 3H), 1.38 (s, 4H), 1.28 (t, J=7.5 Hz, 3H)

### Synthesis of Compound 69

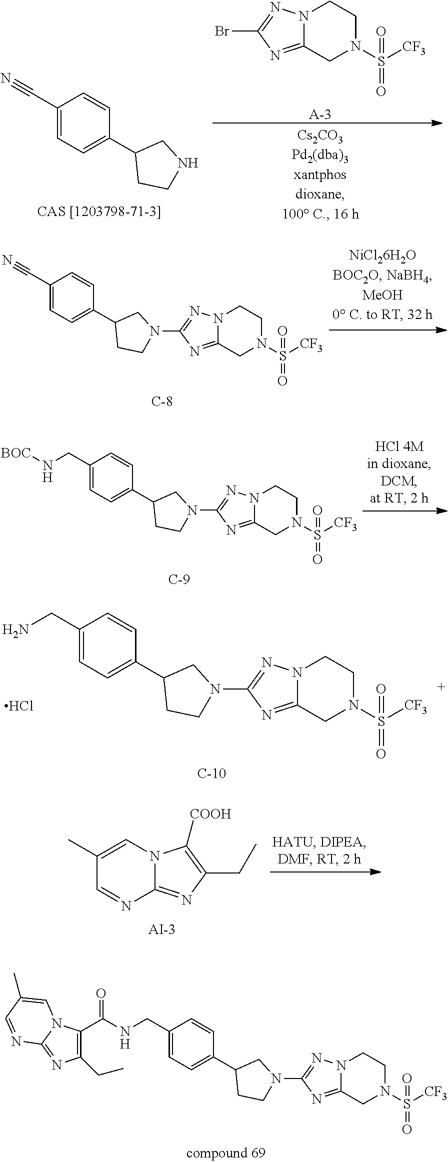

### Preparation of Intermediate C-8

A N₂ purged mixture of 4-pyrrolidin-3-ylbenzonitrile (CAS [1203798-71-3], 155 mg, 0.9 mmol), intermediate A-3 (250 mg, 0.75 mmol), Cesium carbonate (732 mg, 2.25 mmol), Tris(dibenzylideneacetone)dipalladium(0) (102 mg, 0.11 mmol) and 4,5-Bis(diphenylphosphino)-9,9-dimethylxanthene (130 mg, 0.22 mmol) in 1,4-dioxane (7 mL) was stirred at 100° C. for 16 h.

The reaction mixture was cooled down to RT, Ethyl acetate and NaHCO₃ were added to the reaction mixture, the organic layer was separated, dried over MgSO₄, filtered and concentrated under vacuum. The crude was purified by flash column chromatography (dry load in silica, EtOAc in Heptane from 0/100 to 75/25) The desired fractions were collected and concentrated in vacuo to afford intermediate C-8 as a yellow oil, 0.145 g (40%).

### Preparation of Intermediate C-9

To a nitrogen-purged mixture of intermediate C-8 (145 mg, 0.32 mmol), Nickel(II) chloride hexahydrate (58 mg, 0.24 mmol), Di-tertbutyl dicarbonate (211 mg, 0.97) in dry MeOH (3 mL) was added portionwise Sodium borohydride (73 mg, 1.94), mmol) at 0° C. The reaction mixture was stirred at RT for 32 h. A solution of NH₄Cl sat. aqueous was added and the mixture was extracted with DCM (×3). The organic layer was dried with MgSO4, filtrated, and concentrated in vacuo to yield intermediate C-9 as a brown solid, 0.092 g (43%) that it was used in the next step without further purification.

### Preparation of Intermediate C-10

Accordingly, intermediate C-10 was prepared in the same way as intermediate C-3 starting from intermediate C-9 (92 mg, 0.17 mmol) to afford intermediate C-10 as a purple solid, 0.080 g (79%).

### Preparation of Compound 69

Accordingly, compound 69 was prepared in the same way as compound 1 starting from intermediate AI-3 2-ethyl-6-methyl-imidazo[1,2-a]pyrimidine-3-carboxylic acid (0.21 mmol) and intermediate C-10 (0.16 mmol) yielding 40 mg (39%) as a white powder.

1H NMR (400 MHz, DMSO-d6, 25° C.) δ 9.24-9.20 (m, 1H), 8.73-8.65 (m, 2H), 7.33 (d, J=8.3 Hz, 2H), 7.28 (d, J=8.2 Hz, 2H), 4.75 (s, 2H), 4.52 (d, J=5.8 Hz, 2H), 4.07 (s, 4H), 3.76 (dd, J=9.5, 7.5 Hz, 1H), 3.64-3.51 (m, 4H), 3.03 (q, J=7.5 Hz, 2H), 2.39 (s, 3H), 1.98 (dq, J=12.1, 8.4 Hz, 2H), 1.29 (t, J=7.5 Hz, 3H).

1H NMR (400 MHz, DMSO-d6, 100° C.) δ 9.16 (dd, J=2.3, 1.1 Hz, 1H), 8.54 (d, J=2.4 Hz, 1H), 8.15 (s, 1H), 7.35 (d, J=8.1 Hz, 2H), 7.28 (d, J=8.1 Hz, 2H), 4.73 (s, 2H), 4.55 (d, J=5.8 Hz, 2H), 4.08 (dq, J=8.2, 4.2 Hz, 4H), 3.79 (dd, J=9.6, 7.5 Hz, 1H), 3.58-3.40 (m, 3H), 3.36-3.27 (m, 1H), 2.38-2.35 (m, 3H), 2.07-1.95 (m, 2H), 1.31 (t, J=7.5 Hz, 3H). CH2 missed, CH2 inside of water signal.

### Synthesis of Compound 70

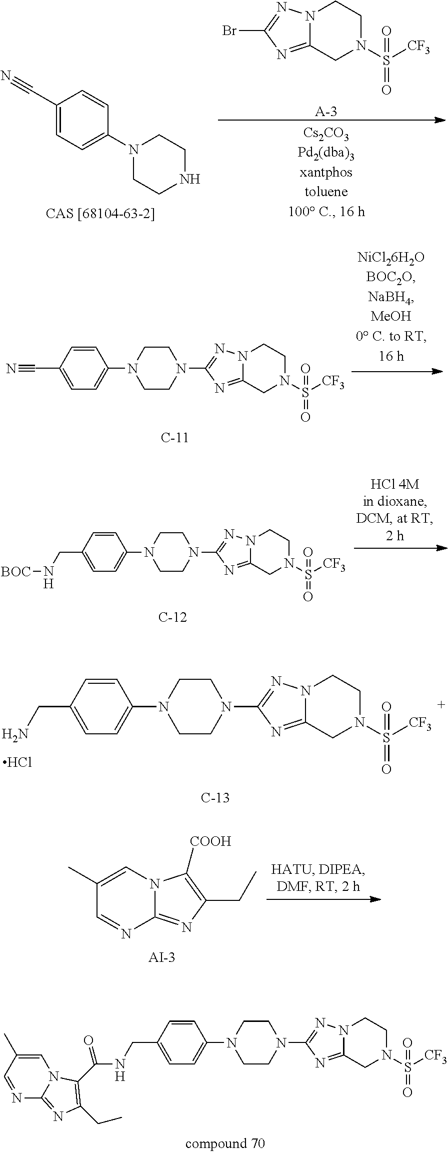

### Preparation of Intermediate C-11

Accordingly, intermediate C-11 was prepared in the same way as intermediate C-8 starting from intermediate 4-piperazin-1-ylbenzonitrile (CAS [68104-63-2], 200 mg, 0.89 mmol),), intermediate A-3 (250 mg, 0.75 mmol), in Toluene (20 mL) to afford intermediate C-11 as a yellow solid, 0.134 g (39%).

### Preparation of Intermediate C-12

Accordingly, intermediate C-12 was prepared in the same way as intermediate C-9 starting from intermediate C-11 (364 mg, 0.82 mmol) to afford intermediate C-12 as a yellow solid, 0.450 g (90%).

### Preparation of Intermediate C-13

Accordingly, intermediate C-13 was prepared in the same way as intermediate C-3 starting from intermediate C-12 (449 mg, 0.74 mmol) to afford intermediate C-13 as a yellow solid, 0.384 g (85%).

### Preparation of Compound 70

Accordingly, compound 70 was prepared in the same way as compound 1 starting from intermediate AI-3 2-ethyl-6-methyl-imidazo[1,2-a]pyrimidine-3-carboxylic acid (0.82 mmol) and intermediate C-13 (0.63 mmol) yielding 136 mg (34%) as a beige solid.

1H NMR (400 MHz, DMSO) δ 9.12 (s, 1H), 8.50 (d, J=2.3 Hz, 1H), 8.40 (t, J=5.9 Hz, 1H), 7.25 (d, J=8.5 Hz, 2H), 6.97 (d, J=8.6 Hz, 2H), 4.77 (s, 2H), 4.43 (d, J=5.8 Hz, 2H), 4.09 (dd, J=12.8, 4.3 Hz, 4H), 3.47-3.40 (m, 4H), 3.22-3.13 (m, 4H), 2.98 (q, J=7.5 Hz, 2H), 2.33 (s, 3H), 1.26 (t, J=7.5 Hz, 3H).

### Synthesis of Compound 71

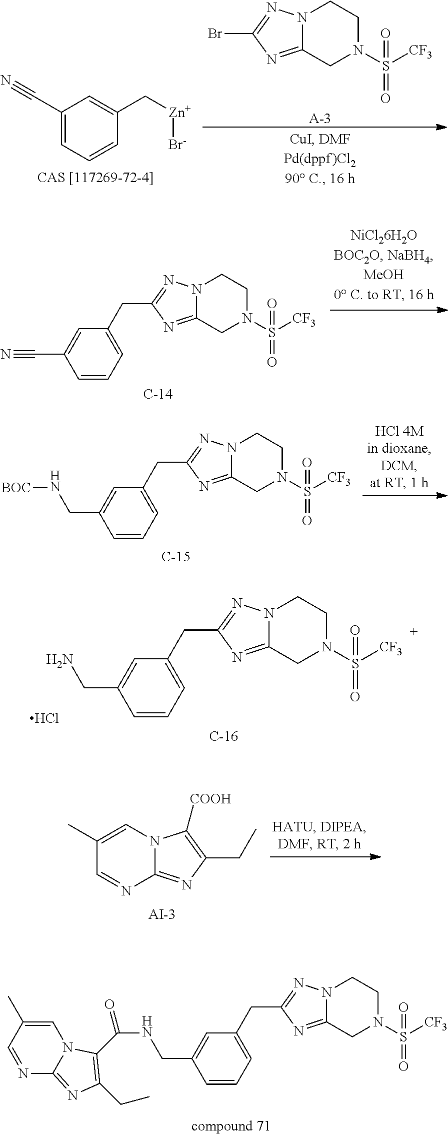

### Preparation of Intermediate C-14

[1,1′-Bis(diphenylphosphino)ferrocene]dichloropalladium (II) complex with dichloromethaneylbenzonitrile (CAS [95464-05-4], 49 mg, 0.06 mmol) and CuI (11 mg, 12 mmol) were added to a stirred solution of intermediate A-3 (200 mg, 0.6 mmol) in DMF (9 mL) in a round bottom flask 2-neck equipped with a condenser at rt while nitrogen was bubbling. Then, (3-Cyanobenzyl)zinc bromide (CAS [117269-72-4], 624 mg, 2.39 mmol, 0.24 M solution in THF) was added via syringe to the stirred suspension under nitrogen. The mixture was stirred at 90° C. for 16 h. The mixture was diluted with water and extracted with AcOEt. The organic layer was separated, dried (MgSO4), filtered and the solvent evaporated in vacuo. The crude product was purified by flash column chromatography (silica; Ethyl acetate in heptane 0/100 to 100/0). The desired fractions were collected and concentrated in vacuo to yield intermediate C-14 as a brown solid 0.080 g (36%).

### Preparation of Intermediate C-15

Accordingly, intermediate C-15 was prepared in the same way as intermediate C-9 starting from intermediate C-14 (80 mg, 0.22 mmol) to afford intermediate C-15 as a brown oil, 0.095 g (83%).

### Preparation of Intermediate C-16

Accordingly, intermediate C-16 was prepared in the same way as intermediate C-3 starting from intermediate C-15 (80 mg, 0.2 mmol) to afford intermediate C-16 as a yellow solid, 0.090 g (90%).

### Preparation of Compound 71

Accordingly, compound 71 was prepared in the same way as compound 1 starting from intermediate AI-3 2-ethyl-6-methyl-imidazo[1,2-a]pyrimidine-3-carboxylic acid (0.24 mmol) and intermediate C-16 (0.2 mmol) yielding 55 mg (47%) as a brown solid.

1H NMR (400 MHz, DMSO) δ 9.13 (s, 1H), 8.55-8.43 (m, 2H), 7.35-7.10 (m, 4H), 4.81 (s, 2H), 4.50 (d, J=5.0 Hz, 2H), 4.22 (s, 2H), 4.16-4.01 (m, 4H), 3.04-2.94 (m, 2H), 2.33 (s, 3H), 1.26 (t, J=7.3 Hz, 3H).

### Synthesis of Compound 72

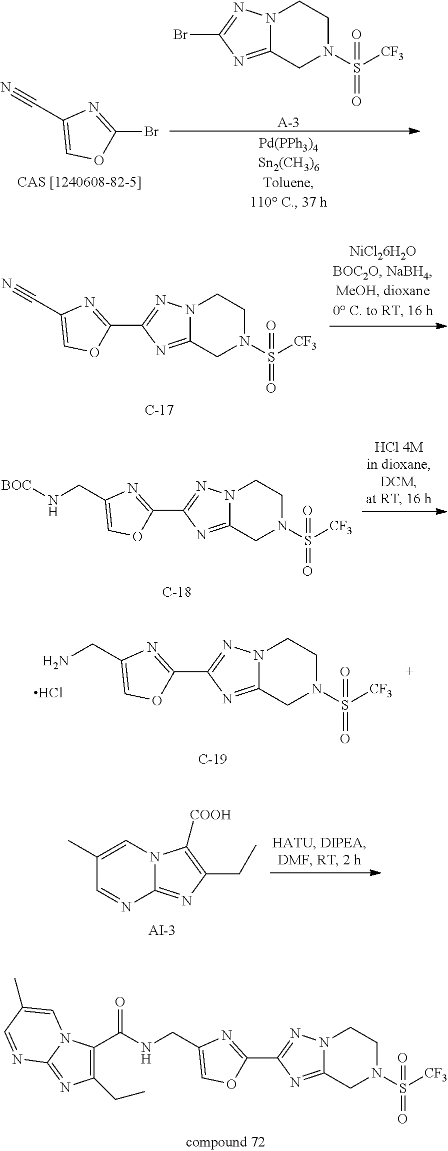

### Preparation of Intermediate C-17

Tetrakis(triphenylphosphine)palladium(0) (CAS [14221-01-3], 334 mg, 0.29 mmol) was added under N2 atmosphere to a mixture of intermediate A3 (484 mg, 1.45 mmol), Hexamethylditin (CAS [661-69-8], 0.3 mL, 1.45 mmol, 1.58 g/mL) in toluene (20 mL). The mixture was stirred at 110° C. for 5 h. Then, 2-bromooxazole-4-carbonitrile (CAS [1240608-82-5], 375 mg, 2.17 mmol) and additional Tetrakis(triphenylphosphine)palladium(0) (CAS [14221-01-3], 334 mg, 0.29 mmol) were added to the reaction mixture and stirred at 110° C. for additional 16 h. Reaction incomplete, Tetrakis(triphenylphosphine)palladium(0) (CAS [14221-01-3], 334 mg, 0.29 mmol) was added at rt and the mixture was stirred at 110° C. for 16h. The mixture was cooled down at rt and filtered through of pad of celite. Solvent was concentrated in vacuo. The reaction crude was purified by flash column chromatography (silica gel, EtOAc in heptane from 0/100 to 100/0). The desired fractions were combined, and the solvent removed in vacuo to yield intermediate C-17 as a yellow solid 362 mg (50%).

### Preparation of Intermediate C-18

Accordingly, intermediate C-18 was prepared in the same way as intermediate C-9 starting from intermediate C-17 (316 mg, 0.91 mmol) to afford intermediate C-18 as a brown oil, 0.434 g (95%).

### Preparation of Intermediate C-19

Accordingly, intermediate C-19 was prepared in the same way as intermediate C-3 starting from intermediate C-18 (434 mg, 0.58 mmol) to afford intermediate C-19 as a yellow solid, 0.372 g (100%).

### Preparation of Compound 72

Accordingly, compound 72 was prepared in the same way as compound 1 starting from intermediate AI-3 2-ethyl-6-methyl-imidazo[1,2-a]pyrimidine-3-carboxylic acid (0.75 mmol) and intermediate C-19 (0.58 mmol) yielding 55 mg (18%) as a beige solid.

1H NMR (400 MHz, DMSO) δ 9.16 (s, 1H), 8.51 (d, J=2.4 Hz, 1H), 8.46 (t, J=5.7 Hz, 1H), 8.17 (s, 1H), 4.98 (s, 2H), 4.50 (d, J=5.6 Hz, 2H), 4.41 (t, J=5.4 Hz, 2H), 4.22-4.11 (m, 2H), 3.00 (q, J=7.5 Hz, 2H), 2.35 (s, 3H), 1.27 (t, J=7.5 Hz, 3H).

### Synthesis of Compound 73

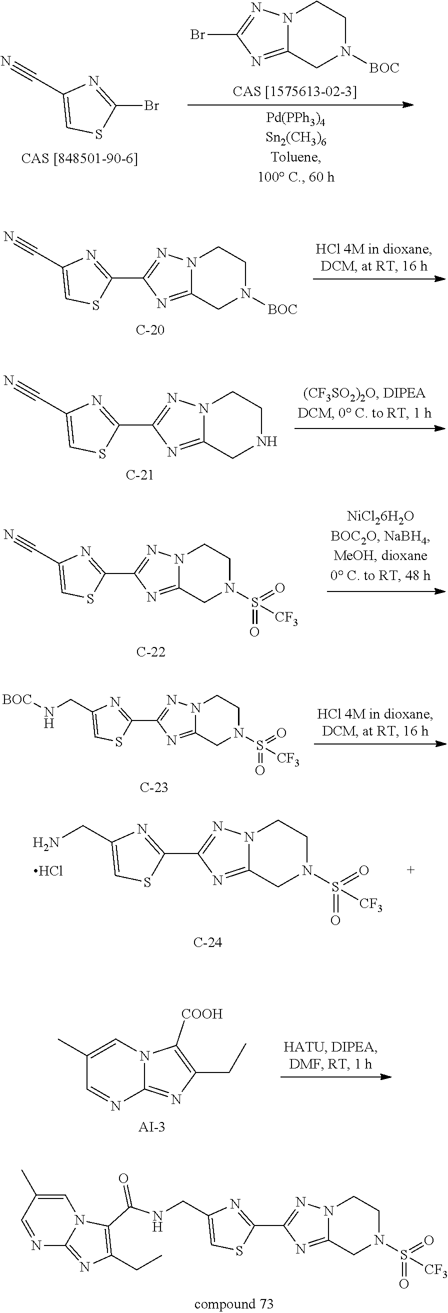

### Preparation of Intermediate C-20

Accordingly, intermediate C-20 was prepared in the same way as intermediate C-17 starting from 2-bromothiazole-4-carbonitrile (CAS [848501-90-6], 280 mg, 1.48 mmol), and tert-butyl 2-bromo-6,8-dihydro-5H-[1,2,4]triazolo[1,5-a]pyrazine-7-carboxylate (CAS [1575613-02-3], 300 mg, 0.99 mmol), to afford intermediate C-20 as a pale yellow solid, 0.165 g (50%).

### Preparation of Intermediate C-21

Accordingly, intermediate C-21 was prepared in the same way as intermediate C-3 starting from intermediate C-20 (165 mg, 0.5 mmol) to afford intermediate C-21 as a white solid, 0.145 g (100%).

### Preparation of Intermediate C-22

Trifluoromethanesulfonic anhydride (CAS [358-23-6], 0.100 mL, 0.59 mmol), was added dropwise to a stirred solution of intermediate C-21 (145 mg, 0.54 mmol), DIPEA (0.282 mL, 1.60 mmol) in DCM (6 mL) in a round bottom flask under N2 atmosphere at 0° C. The mixture was stirred for 30 min at 0° C. and 1 h at rt. Aqueous saturated NaHCO3 solution was added and the mixture was extracted with DCM. The combined organic layers were dried over MgSO4, filtered, and concentrated in vacuo. The crude product was purified by flash column chromatography (silica; ethyl acetate in heptane from 0/100 to 30/70). The desired fractions were collected and concentrated in vacuo to yield intermediate C-22 as a white solid 0.077 g (39%).

### Preparation of Intermediate C-23

Accordingly, intermediate C-23 was prepared in the same way as intermediate C-9 starting from intermediate C-22 (75 mg, 0.21 mmol) to afford intermediate C-23 as a brown solid, 83 mg (86%).

### Preparation of Intermediate C-24

Accordingly, intermediate C-24 was prepared in the same way as intermediate C-3 starting from intermediate C-23 (83 mg, 0.18 mmol) to afford intermediate C-24 as a yellow solid, 87 mg (99%).

### Preparation of Compound 73

Accordingly, compound 73 was prepared in the same way as compound 1 starting from intermediate AI-3 2-ethyl-6-methyl-imidazo[1,2-a]pyrimidine-3-carboxylic acid (0.19 mmol) and intermediate C-24 (0.18 mmol) yielding 15 mg (10%) as a yellow solid.

1H NMR (400 MHz, DMSO) δ 9.18-9.15 (m, 1H), 8.57 (t, J=5.9 Hz, 1H), 8.51 (d, J=2.4 Hz, 1H), 7.62 (s, 1H), 4.98 (s, 2H), 4.69 (d, J=5.8 Hz, 2H), 4.39 (t, J=5.4 Hz, 2H), 4.17 (t, J=5.1 Hz, 2H), 3.02 (q, J=7.5 Hz, 2H), 2.34 (s, 3H), 1.28 (t, J=7.5 Hz, 3H).

### Synthesis of Compound 74

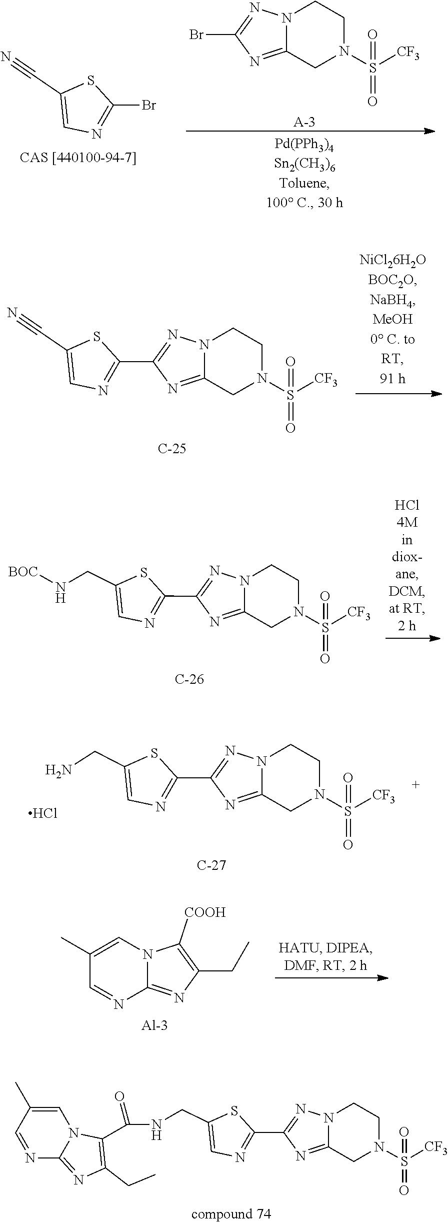

### Preparation of Intermediate C-25

Accordingly, intermediate C-25 was prepared in the same way as intermediate C-17 starting from 2-bromothiazole-5-carbonitrile (CAS [440100-94-7], 500 mg, 2.54 mmol), and intermediate A3 (567 mg, 1.69 mmol), to afford intermediate C-25 as a pale brown solid, 0.180 g (26%).

### Preparation of Intermediate C-26

Accordingly, intermediate C-26 was prepared in the same way as intermediate C-9 starting from intermediate C-25 (233 mg, 0.64 mmol) to afford intermediate C-26 as a brown oil, 0.299 g (85%).

### Preparation of Intermediate C-27

Accordingly, intermediate C-27 was prepared in the same way as intermediate C-3 starting from intermediate C-26 (299 mg, 0.54 mmol) to afford intermediate C-27 as a yellow solid, 0.280 g (58%).

### Preparation of Compound 74

Accordingly, compound 74 was prepared in the same way as compound 1 starting from intermediate AI-3 2-ethyl-6-methyl-imidazo[1,2-a]pyrimidine-3-carboxylic acid (0.38 mmol) and intermediate C-27 (0.32 mmol) yielding 38 mg (21%) as a brown solid.

1H NMR (400 MHz, DMSO) δ 9.19 (dd, J=2.3, 1.1 Hz, 1H), 8.61 (t, J=5.8 Hz, 1H), 8.53 (d, J=2.4 Hz, 1H), 7.89 (s, 1H), 4.97 (s, 2H), 4.75 (d, J=5.7 Hz, 2H), 4.39 (t, J=5.4 Hz, 2H), 4.16 (d, J=5.1 Hz, 2H), 2.99 (q, J=7.5 Hz, 2H), 2.35 (s, 3H), 1.26 (t, J=7.5 Hz, 3H).

### Synthesis of Compound 75

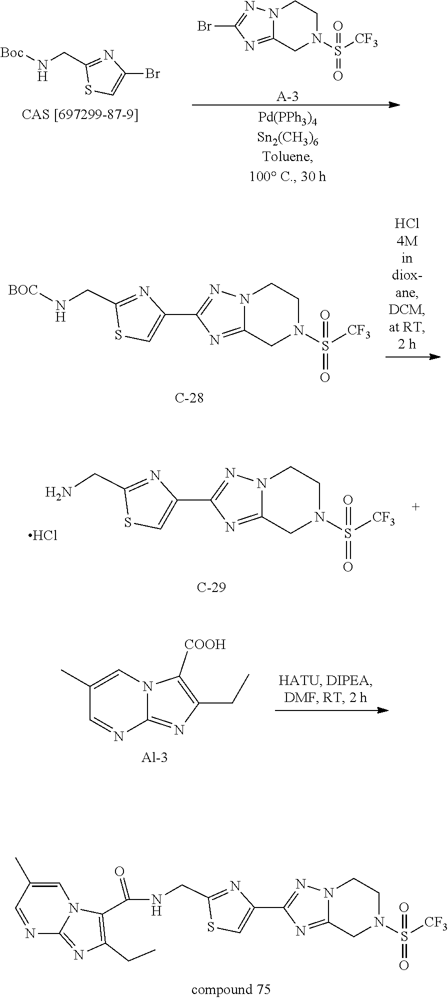

### Preparation of Intermediate C-28

Accordingly, intermediate C-28 was prepared in the same way as intermediate C-17 starting from tert-butyl N-[(4-bromothiazol-2-yl)methyl]carbamate (CAS [697299-87-9], 750 mg, 2.56 mmol), and intermediate A3 (571 mg, 1.71 mmol), to afford intermediate C-28 as a yellow oil, 0.403 g (29%).

### Preparation of Intermediate C-29

Accordingly, intermediate C-29 was prepared in the same way as intermediate C-3 starting from intermediate C-28 (403 mg, 0.49 mmol) to afford intermediate C-29 as a yellow solid, 0.360 g (100%).

### Preparation of Compound 75

Accordingly, compound 75 was prepared in the same way as compound 1 starting from intermediate AI-3 2-ethyl-6-methyl-imidazo[1,2-a]pyrimidine-3-carboxylic acid (0.69 mmol) and intermediate C-29 (0.49 mmol) yielding 32 mg (12%) as a beige solid.

1H NMR (400 MHz, DMSO) δ 9.19 (s, 1H), 8.84 (t, J=5.9 Hz, 1H), 8.54 (d, J=2.4 Hz, 1H), 8.06 (s, 1H), 4.95 (s, 2H), 4.86 (d, J=5.9 Hz, 2H), 4.36 (t, J=5.4 Hz, 2H), 4.17 (t, J=5.3 Hz, 2H), 3.07 (q, J=7.5 Hz, 2H), 2.35 (s, 3H), 1.32 (t, J=7.5 Hz, 3H).

### Synthesis of Compound 76

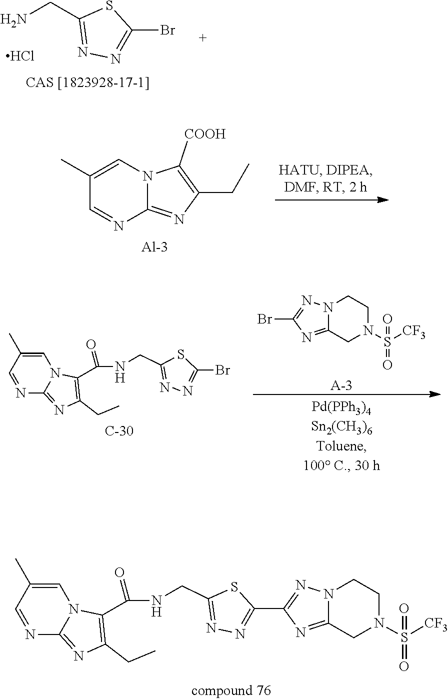

### Preparation of Intermediate C-30

Accordingly, intermediate C-30 was prepared in the same way as compound 1 starting from intermediate AI-3 2-ethyl-6-methyl-imidazo[1,2-a]pyrimidine-3-carboxylic acid (158 mg 0.77 mmol) and (5-bromo-1,3,4-thiadiazol-2-yl)methanamine hydrochloride (CAS [1823928-17-1], 187 mg 0.7 mmol) yielding 260 mg (68%) as a brown solid.

### Preparation of Compound 76

Accordingly, compound 76 was prepared in the same way as intermediate C-17 starting from intermediate C-30 (180 mg, 0.33 mmol), and intermediate A3 (221 mg, 0.66 mmol), yielding 38 mg (20%) as a beige solid.

1H NMR (400 MHz, DMSO) δ 9.22 (s, 1H), 8.85 (s, 1H), 8.61-8.47 (m, 1H), 5.00 (s, 2H), 4.97 (s, 2H), 4.43 (t, J=5.4 Hz, 2H), 4.17 (m, 2H), 3.04 (q, J=7.5 Hz, 2H), 2.35 (s, 3H), 1.29 (t, J=7.5 Hz, 3H).

### Synthesis of Compound 77

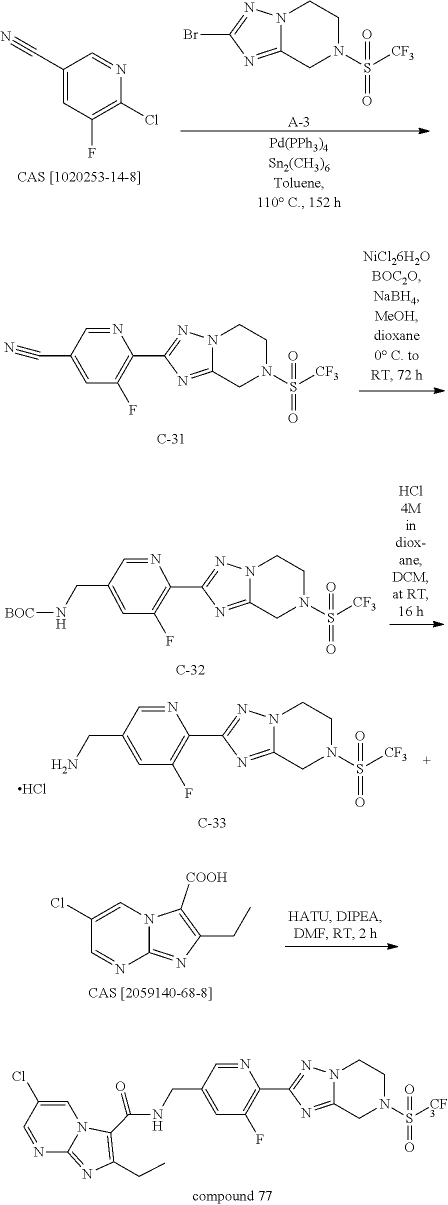

### Preparation of Intermediate C-31

Accordingly, intermediate C-31 was prepared in the same way as intermediate C-17 starting from 6-chloro-5-fluoro-pyridine-3-carbonitrile (CAS [1020253-14-8], 1 g, 6.39 mmol), and intermediate A3 (713 mg, 2.13 mmol), to afford intermediate C-31 as a yellow oil, 0.234 g (12%).

### Preparation of Intermediate C-32

Accordingly, intermediate C-32 was prepared in the same way as intermediate C-9 starting from intermediate C-31 (257 mg, 0.68 mmol) to afford intermediate C-32 as a brown solid 0.276 g (54%).

### Preparation of Intermediate C-33

Accordingly, intermediate C-33 was prepared in the same way as intermediate C-3 starting from intermediate C-32 (275 mg, 0.57 mmol) to afford intermediate C-33 as a yellow solid, 0.285 g (99%).

### Preparation of Compound 77

Accordingly, compound 77 was prepared in the same way as compound 1 starting from 6-chloro-2-ethyl-imidazo[1,2-a]pyrimidine-3-carboxylic acid (CAS [2059140-68-8], 0.74 mmol) and intermediate C-33 (0.57 mmol) yielding 38 mg (12%) as a brown solid.

1H NMR (400 MHz, DMSO) δ 9.44 (d, J=2.6 Hz, 1H), 8.69 (d, J=2.6 Hz, 1H), 8.64 (t, J=6.0 Hz, 1H), 8.58 (s, 1H), 7.87 (d, J=10.9 Hz, 1H), 4.99 (s, 2H), 4.66 (d, J=5.6 Hz, 2H), 4.42 (t, J=5.3 Hz, 2H), 4.18 (s, 2H), 3.06 (q, J=7.5 Hz, 2H), 1.30 (t, J=7.5 Hz, 3H).

### Synthesis of Compound 78

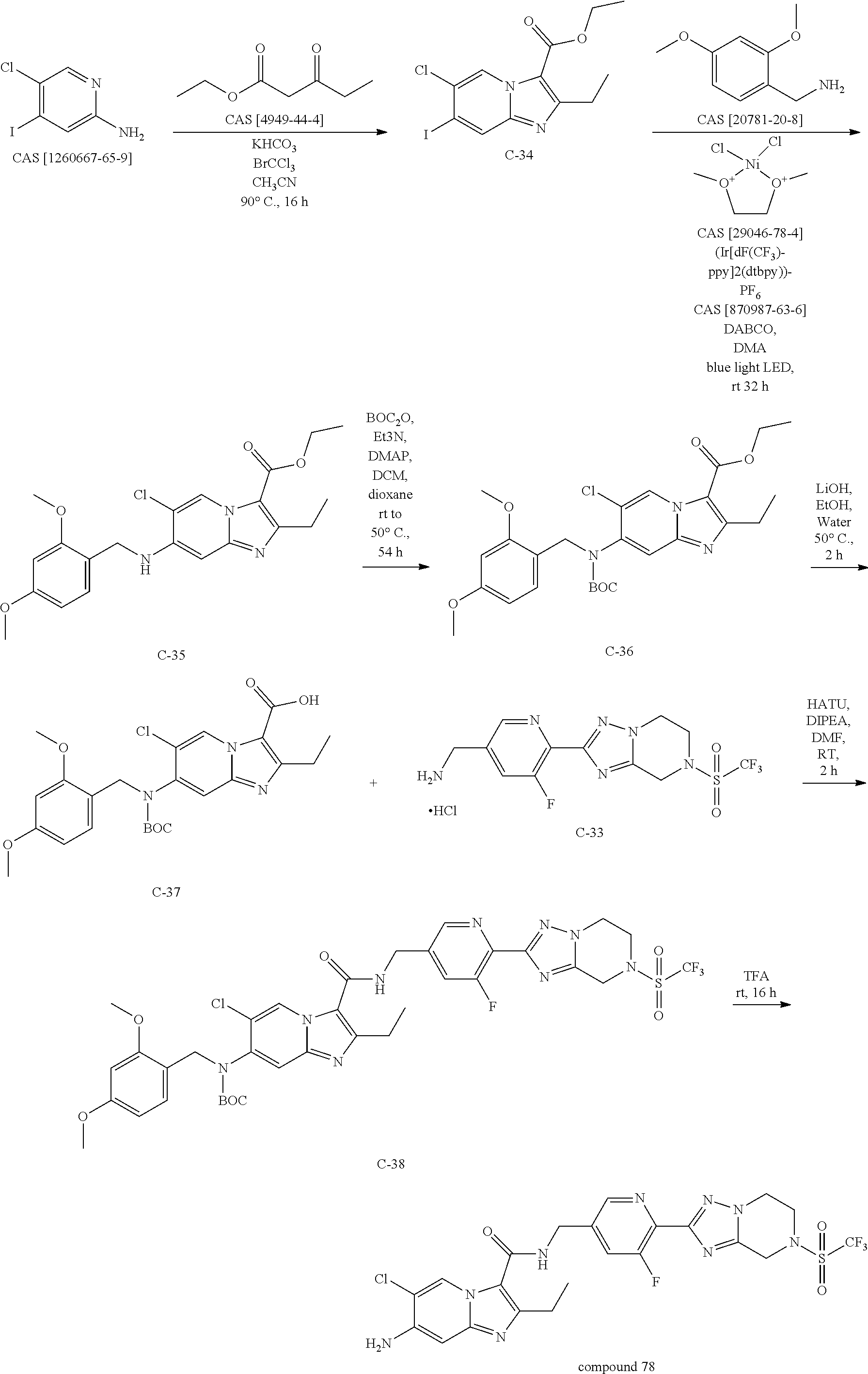

### Preparation of Intermediate C-34

Ethyl propionylacetate (CAS [4949-44-4], 0.100 mL, 0.59 mmol), was added to a stirred mixture of 5-Chloro-4-iodopyridin-2-amine (CAS [1260667-65-9], 3.6 g, 14.15 mmol), KHCO₃ (3.1 g, 31.13 mmol), Bromotrichloromethane (CAS [75-62-7], 5.5 g, 56.59 mmol), in Acetonitrile (10 mL) at rt. The mixture was stirred at 90° C. for 16 hours. Then, the mixture was diluted with EtOAc and washed with sat. NaHCO3 aq. solution. The organic layer was separated, dried over MgSO₄, filtered, and concentrated in vacuo. The crude was purified by flash column chromatography (silica; EtOAc/Heptane from 0/100 to 25/75). The desired fractions were collected, and the solvent evaporated in vacuo to yield intermediate C-34 1.13 g, (20%) as a yellow powder.

### Preparation of Intermediate C-35

In a screw top vial, a solution of Nickel(II) chloride ethylene glycol dimethyl ether complex (CAS [29046-78-4], 105 mg, 0.48 mmol), in DMA (1 mL) was added to a mixture of intermediate C-34, 2,4-Dimethoxybenzylamine (0.7 mL), (Ir[dF(CF3)ppy]2(dtbpy))PF6 (CAS [870987-63-6], 54 mg, 0.44 mmol), in DMA (8 mL) under nitrogen at rt. The mixture was degassed with nitrogen, the vial was closed, and the mixture stirred at rt and irradiated with blue light LED for 32 h. The mixture was diluted with saturated NaHCO3 aqueous solution and extracted with AcOEt. The organic layer was dried over MgSO4, filtered and concentrated in vacuo.

The crude product was purified by flash column chromatography (silica; AcOEt/heptane from 0/100 to 70/30). The desired fractions were collected, and the solvent evaporated in vacuo to yield intermediate C-35 0.250 g, (24%) as a yellow solid.

### Preparation of Intermediate C-36

In a round bottom flask, Di-tertbutyl decarbonate (0.5 g, 2.34 mmol) was added to a solution of intermediate C-35 (0.245 g, 0.59 mmol), Triethylamine (0.6 mL, 4.39 mmol), and DMAP (3.58 mg, 0.029 mmol), in DCM (2 mL) at rt. The mixture was stirred at rt for 16h. The reaction mixture was concentrated in vacuo and dioxane was added (2 mL). The reaction mixture was stirred at 100° C. for 16h.

The reaction mixture was diluted with water and brine solution and extracted with DCM. The organic layer was dried over anhydrous MgSO4, filtered, and concentrated in vacuo. The crude product was purified by flash column chromatography (silica, AcOEt in DCM from 0/100 to 40/60)). The desired fractions were collected and concentrated in vacuo to yield intermediate C-36, 0.270 g, (88%) as a beige solid.

### Preparation of Intermediate C-37

To a solution of intermediate C-36 (270 mg, 0.52 mmol) in water (2.5 mL) and EtOH (9 mL) was added LiOH (66 mg, 1.56 mmol). The reaction mixture was stirred for 2 h at 50° C. Then HCl 1 M aq. solution was added until pH 7, and the solvent was evaporated in vacuo to yield intermediate C-37, 0.280 g, (100%) as an orange solid. The reaction mixture was used in the next step without any further purification.

### Preparation of Intermediate C-38

Accordingly, intermediate C-38 was prepared in the same way as compound 1 starting from intermediate C-37 (277 mg, 0.52 mmol) and intermediate C-33 (472 mg, 1.04 mmol) yielding 113 mg (12%) as a yellow foam.

### Preparation of Compound 78

TFA (1.13 mL) was added to intermediate C-38 (100 mg, 0.12 mmol) at 0° C. The mixture was stirred at rt for 16 h. The mixture was neutralized with sat. aqueous NaHCO3 solution and extracted with DCM. The organic layer was separated, dried over anhydrous MgSO4, filtered, and concentrated in vacuo. The crude was purified by flash column chromatography (silica; DCM/MeOH (9:1) in DCM from 0/100 to 60/40). The desired fractions were collected, and the solvent was evaporated in vacuo. Diethylether and pentane were added and dried under vacuo to yield compound 78, 26 mg (37%) as a white solid.

1H NMR (400 MHz, DMSO) δ 9.05 (s, 1H), 8.55 (s, 1H), 8.08 (t, J=5.9 Hz, 1H), 7.81 (d, J=11.4 Hz, 1H), 6.64 (s, 1H), 6.13 (s, 2H), 4.99 (s, 2H), 4.60 (d, J=5.8 Hz, 2H), 4.42 (t, J=5.4 Hz, 2H), 4.18 (t, J=5.1 Hz, 2H), 3.00-2.84 (m, 2H), 1.24 (t, J=7.5 Hz, 3H).

### Synthesis of Compound 79 and 80

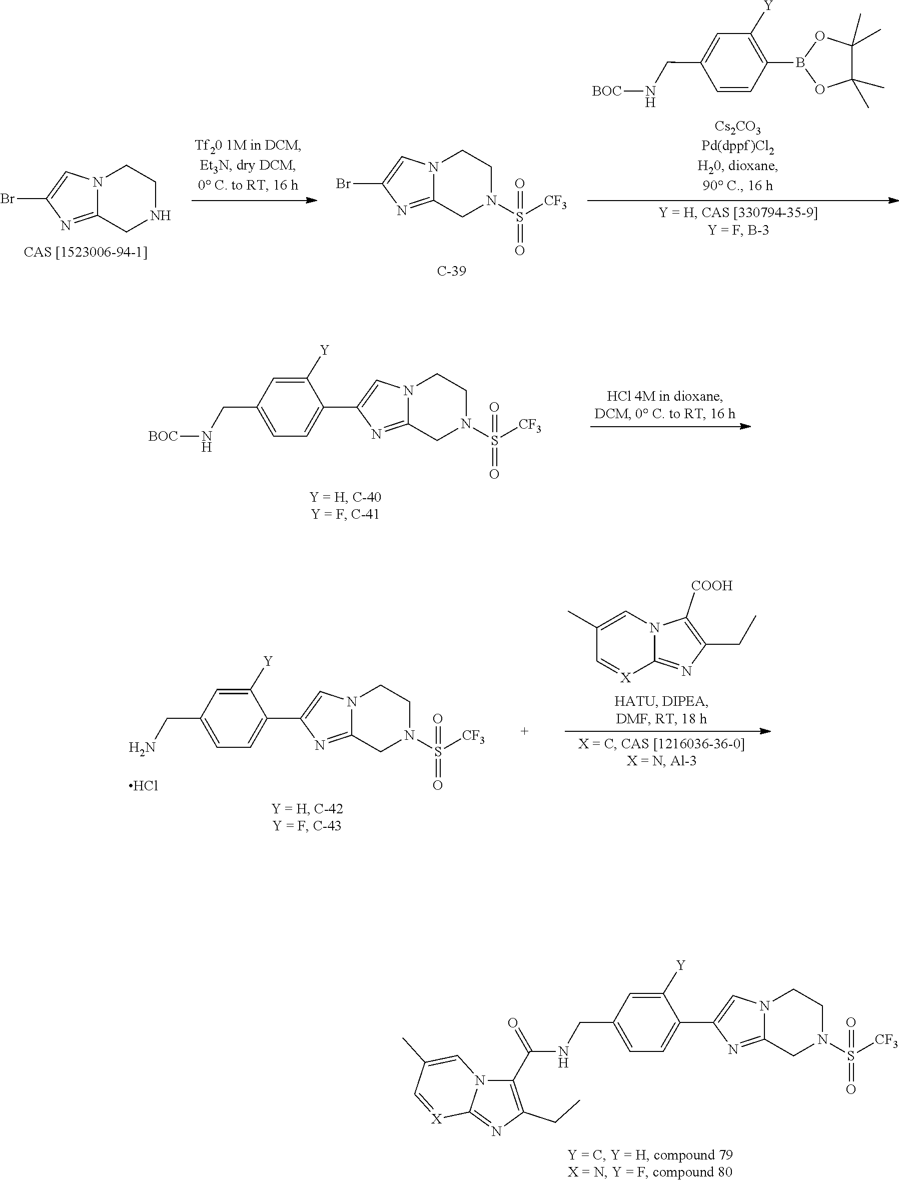

### Preparation of Intermediate C-39

Accordingly, intermediate C-39 was prepared in the same way as intermediate A-3 starting from intermediate 2-bromo-5,6,7,8-tetrahydroimidazo[1,2-a]pyrazine (CAS [1523006-94-1], 823 mg, 4.07 mmol), to afford intermediate C-39 as a yellow solid, 0.606 g (44%).

### Preparation of Intermediate C-40

Accordingly, intermediate C-40 was prepared in the same way as intermediate C-2 starting from intermediate C-39 (1.87 mmol) and tert-butyl N-[[4-(4,4,5,5-tetramethyl-1,3,2-dioxaborolan-2-yl)phenyl]methyl]carbamate (CAS [330794-35-9], 2.62 mmol) affording 0.607 g (63%) as yellow solid.

### Preparation of Intermediate C-41

Accordingly, intermediate C-41 was prepared in the same way as intermediate C-40 starting from intermediate C-39 (0.37 mmol) and intermediate B-3 (0.52 mmol) affording 133 mg (74%) as beige solid.

### Preparation of Intermediate C-42

Accordingly, intermediate C-42 was prepared in the same way as intermediate C-3 starting from intermediate C-40 (607 mg, 1.32 mmol) to afford intermediate C-42 as a white solid, 0.580 g (91%).

### Preparation of Intermediate C-43

Accordingly, intermediate C-43 was prepared in the same way as intermediate C-42 starting from intermediate C-41 (133 mg, 0.28 mmol) to afford intermediate C-43 as a white solid, 0.116 g (99%).

### Preparation of Compound 79

Accordingly, compound 79 was prepared in the same way as compound 1 starting from 2-ethyl-6-methyl-imidazo[1,2-a]pyridine-3-carboxylic acid (CAS [1216036-36-0], 0.42 mmol) and intermediate C-42 (0.3 mmol) yielding 0.050 g (29%) as a yellow powder.

1H NMR (400 MHz, DMSO) δ 8.79 (s, 1H), 8.37 (t, J=5.8 Hz, 1H), 7.71 (d, J=8.0 Hz, 2H), 7.67 (s, 1H), 7.51 (d, J=9.1 Hz, 1H), 7.35 (d, J=8.1 Hz, 2H), 7.24 (d, J=9.1 Hz, 1H), 4.79 (s, 2H), 4.52 (d, J=5.9 Hz, 2H), 4.17 (t, J=5.3 Hz, 2H), 4.04 (t, J=7.1 Hz, 2H), 2.97 (q, J=7.5 Hz, 2H), 2.31 (s, 3H), 1.25 (t, J=7.5 Hz, 3H)

### Preparation of Compound 80

Accordingly, compound 80 was prepared in the same way as compound 1 starting from intermediate AI-3 (0.44 mmol) and intermediate C-43 (0.28 mmol) yielding 0.043 g (27%) as a brown solid.

1H NMR (400 MHz, DMSO) δ 9.09 (s, 1H), 8.45 (d, J=2.4 Hz, 1H), 8.41 (t, J=6.0 Hz, 1H), 7.90 (t, J=8.1 Hz, 1H), 7.51 (d, J=4.1 Hz, 1H), 7.19 (s, 1H), 7.16 (d, J=3.9 Hz, 1H), 4.75 (s, 2H), 4.48 (d, J=5.9 Hz, 2H), 4.13 (t, J=5.4 Hz, 2H), 4.03-3.96 (m, 2H), 2.96 (q, J=7.5 Hz, 2H), 2.27 (s, 3H), 1.22 (t, J=7.5 Hz, 3H).

### Synthesis of Compound 81

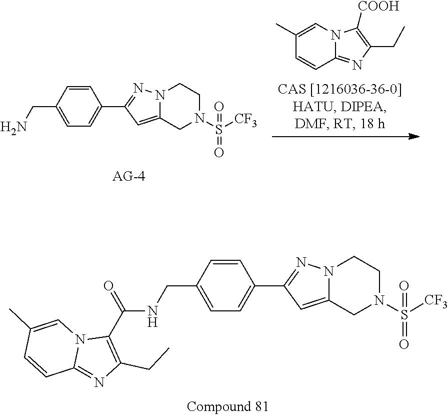 [SMILES: CCc1nc2ccc(C)cn2c1C(=O)O ; NCc1ccc(-c2cc3n(n2)CCN(S(=O)(=O)C(F)(F)F)C3)cc1 ; CCc1nc2ccc(C)cn2c1C(=O)NCc1ccc(-c2cc3n(n2)CCN(S(=O)(=O)C(F)(F)F)C3)cc1]

### Preparation of Compound 81

Accordingly, compound 81 was prepared in the same way as compound 1 starting from intermediate AG-4 (0.35 mmol) and 2-ethyl-6-methyl-imidazo[1,2-a]pyridine-3-carboxylic acid (CAS [1216036-36-0], 0.53 mmol) yielding 0.034 g (18%) as a white foam.

1H NMR (400 MHz, DMSO) δ 8.80 (s, 1H), 8.39 (t, J=5.9 Hz, 1H), 7.75 (d, J=8.1 Hz, 2H), 7.51 (d, J=9.1 Hz, 1H), 7.41 (d, J=8.1 Hz, 2H), 7.24 (dd, J=9.1, 1.3 Hz, 1H), 6.63 (s, 1H), 4.87 (s, 2H), 4.54 (d, J=5.9 Hz, 2H), 4.28 (t, J=5.5 Hz, 2H), 4.10 (t, J=4.9 Hz, 2H), 2.98 (q, J=7.5 Hz, 2H), 2.31 (s, 3H), 1.26 (t, J=7.5 Hz, 3H).

### Synthesis of Compound 82

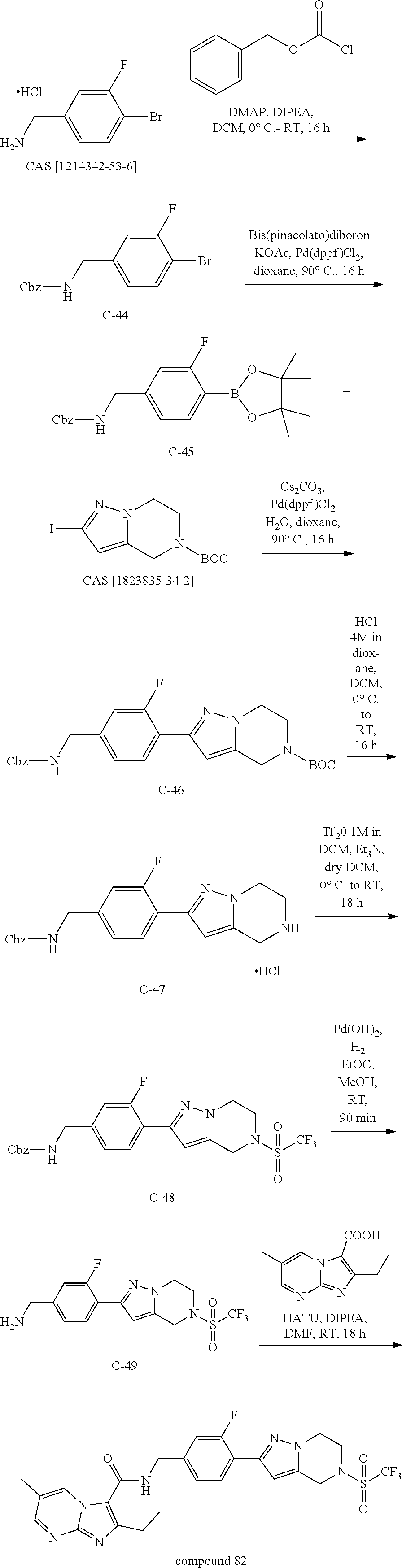

### Preparation of Intermediate C-44

DMAP (CAS [1122-58-3], 25 mg, 0.21 mmol) and DIPEA (CAS [7087-68-5], 1.45 mL, 8.32 mmol) were added to a stirred solution of (4-Bromo-3-fluorophenyl) methanamine hydrochloride (CAS [1214342-53-6], 500 mg, 2.08 mmol) in DCM (21 mL) in a round bottom flask at 0° C. Then Benzyl chloroformate (CAS [501-53-1], 0.45 mL, 3.12 mmol, 1.2 g/mL) was added dropwise at 0° C. The mixture was stirred at rt for 16 h. The mixture was diluted with DCM and aqueous saturated NaHCO3 solution was added. The organic layer was separated, dried over MgSO4, filtered and concentrated in vacuo. The crude product was purified flash column chromatography (silica, EtOAc in Heptane (0/100 to 20/80)). The desired fractions were collected and concentrated in vacuo to yield intermediate C-44, as a white solid, 625 mg (77%).

### Preparation of Intermediate C-45

Accordingly, intermediate C-45 was prepared in the same way as intermediate C-1 starting from intermediate C-44 (1.6 g, 4.73 mmol), affording intermediate C-45 as a yellow solid, 1.6 g (82%).

### Preparation of Intermediate C-46

Accordingly, intermediate C-46 was prepared in the same way as intermediate C-41 starting from intermediate C-45 (0.675 g, 1.75 mmol), and tert-butyl 2-iodo-6,7-dihydro-4H-pyrazolo[1,5-a]pyrazine-5-carboxylate (CAS [1823835-34-2], 510 mg, 1.46 mmol) affording intermediate C-46 as a white solid, 0.428 g (61%).

### Preparation of Intermediate C-47

Accordingly, intermediate C-47 was prepared in the same way as intermediate C-3 starting from intermediate C-46 (428 mg, 0.89 mmol) to afford intermediate C-47 as an orange solid, 0.370 g (99%).

### Preparation of Intermediate C-48

Accordingly, intermediate C-48 was prepared in the same way as intermediate A-3 starting from intermediate C-47 (370 mg, 0.89 mmol), to afford intermediate C-48 as a white solid, 0.243 g (53%).

### Preparation of Intermediate C-49

Accordingly, intermediate C-49 was prepared in the same way as intermediate AE-2 starting from C-48 (243 mg, 0.47 mmol), yielding 0.190 g (95%) as white solid.

### Preparation of Compound 82

Accordingly, compound 82 was prepared in the same way as compound 1 starting from intermediate AI-3 (0.73 mmol) and intermediate C-49 (190 mg, 0.46 mmol) yielding 0.189 g (72%) as a beige solid.

1H NMR (400 MHz, DMSO) δ 9.21-9.11 (m, 1H), 8.56-8.43 (m, 2H), 7.89 (t, J=8.2 Hz, 1H), 7.28 (d, J=4.4 Hz, 1H), 7.26 (s, 1H), 6.59 (d, J=3.9 Hz, 1H), 4.90 (s, 2H), 4.56 (d, J=5.9 Hz, 2H), 4.30 (t, J=5.5 Hz, 2H), 4.11 (t, J=5.4 Hz, 2H), 3.03 (q, J=7.5 Hz, 2H), 2.34 (s, 3H), 1.29 (t, J=7.5 Hz, 3H).

### Synthesis of Compound 84

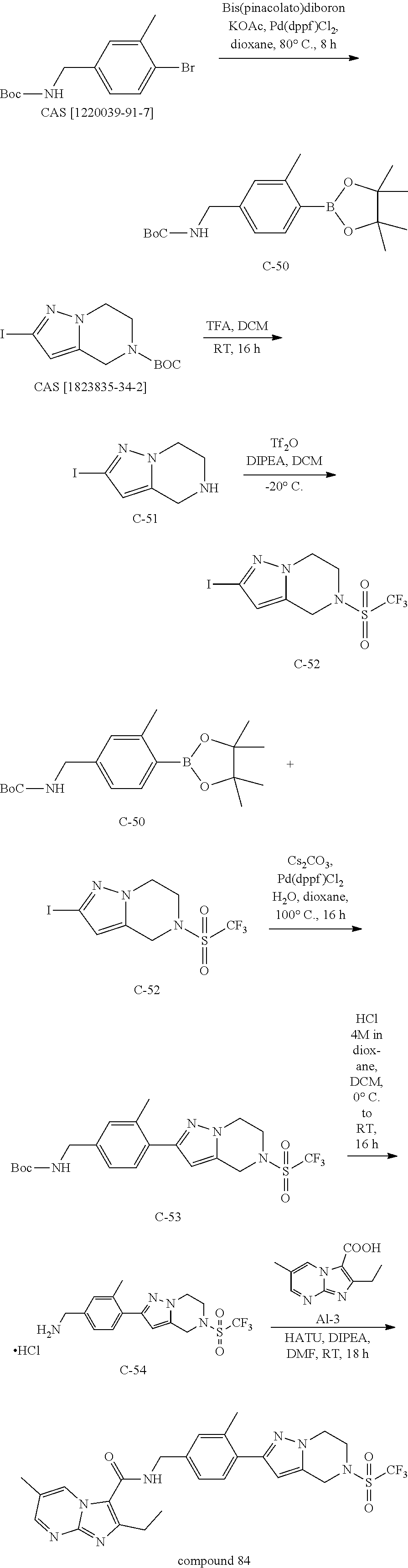

### Preparation of Intermediate C-50

Accordingly, intermediate C-50 was prepared in the same way as intermediate C-45 starting from 1,1-dimethylethyl N-[(4-bromo-3-methylphenyl)methyl]carbamate (CAS [1220039-91-7], 0.640 g, 2.13 mmol) affording intermediate C-50 as a pale yellow oil, 0.740 g (90%).

### Preparation of Intermediate C-51

TFA (0.26 mL, 3.44 mmol) was added to a solution of tert-butyl 2-iodo-6,7-dihydro-4H-pyrazolo[1,5-a]pyrazine-5-carboxylate (CAS [1823835-34-2], 100 mg, 0.29 mmol) in DCM (5 mL) in a round bottom flask at 0° C. The reaction mixture was stirred at rt for 16 h. NaHCO3 saturated aqueous solution was added and the mixture extracted several times with DCM (6×20 mL). The organic phase was dried over MgSO4, filtered, and the solvent removed in vacuo. The crude was purified by flash column chromatography (silica, DCM/MeOH (9:1) in DCM from 0/100 to 100/0). The desired fractions were joined, the solvents removed in vacuo to give intermediate C-51 as a colorless oil, 60 mg (79%).

### Preparation of Intermediate C-52

Accordingly, intermediate C-52 was prepared in the same way as intermediate A-3 starting from intermediate C-51 (60 mg, 0.24 mmol), to afford intermediate C-52 as a pale yellow solid, 80 mg (70%).

### Preparation of Intermediate C-53

Accordingly, intermediate C-53 was prepared in the same way as intermediate C-41 starting from intermediate C-50 (113 mg, 0.33 mmol) and intermediate C-52 (141 mg, 0.3 mmol) affording intermediate C-53 as a colorless oil, 131 mg (84%).

### Preparation of Intermediate C-54

Accordingly, intermediate C-54 was prepared in the same way as intermediate C-3 starting from intermediate C-53 (128 mg, 0.27 mmol) to afford intermediate C-54, 95 mg (77%).

### Preparation of Compound 84

Accordingly, compound 84 was prepared in the same way as compound 1 starting from intermediate AI-3 (66.4 mg, 0.29 mmol) and intermediate C-54 (92 mg, 0.22 mmol) yielding 73 mg (56%) as an off white solid.

### Synthesis of Compound 85

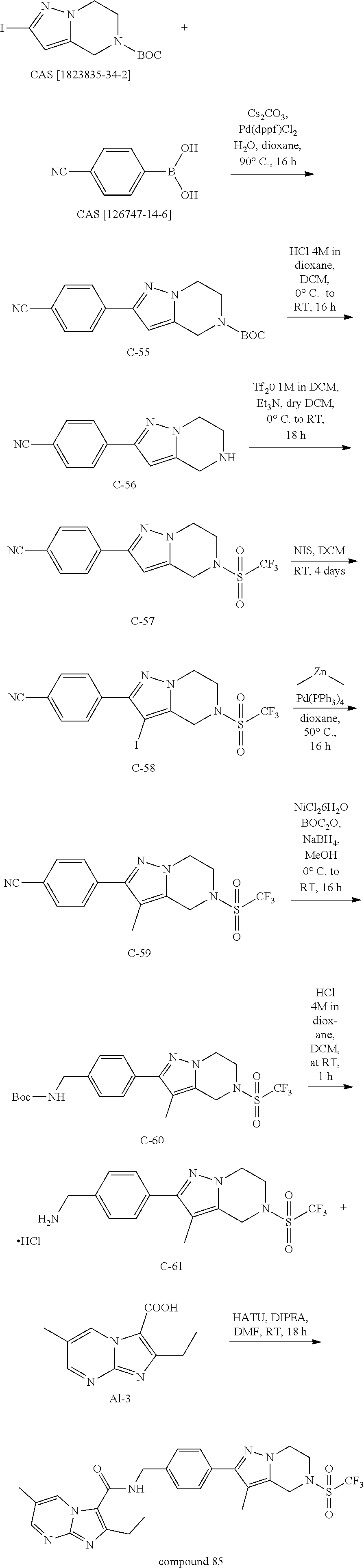

### Preparation of Intermediate C-55

Accordingly, intermediate C-55 was prepared in the same way as intermediate C-41 starting from tert-butyl 2-iodo-6,7-dihydro-4H-pyrazolo[1,5-a]pyrazine-5-carboxylate (CAS [1823835-34-2], 2 g, 5.73 mmol) and 4-cyanophenylboronic acid (CAS [126747-14-6], 1.01 g, 6.87 mmol) affording intermediate C-55 as a white solid, 1.38 g (74%).

### Preparation of Intermediate C-56

Accordingly, intermediate C-56 was prepared in the same way as intermediate C-3 starting from intermediate C-55 (1.38 g, 4.26 mmol) affording intermediate C-56 as a white solid, 1.26 g (quant.).

### Preparation of Intermediate C-57

Accordingly, intermediate C-57 was prepared in the same way as intermediate A-3 starting from intermediate C-56 (1.26 g, 4.26 mmol) affording intermediate C-57 as a white solid, 0.68 g (43%).

### Preparation of Intermediate C-58

N-Iodosuccinimide (325 mg, 1.44 mmol) in DCM (2 mL) was added dropwise to a stirred solution of intermediate C-57 (468 mg, 1.31 mmol) in DCM (13 mL) at rt. The reaction mixture was stirred at rt for 16h. Then, more N-Iodosuccinimide (296 mg, 1.31 mmol) was added at rt and the mixture was stirred at rt for 3h. Then, more N-Iodosuccinimide (148 mg, 0.66 mmol) was added at rt and the mixture was stirred at rt for 72h. Water was added and extracted with DCM. The organic layer was dried over MgSO4, filtered and concentrated in vacuo. The crude product was purified by flash column chromatography (silica; EtOAc in heptane 0/100 to 30/70). The desired fractions were collected, concentrated in vacuo to yield intermediate C-58 as a white solid, 484 mg (76%)

### Preparation of Intermediate C-59

Intermediate C-58 (484 mg, 1 mmol) was added to a flask containing Pd(PPh3)4 (116 mg, 0.1 mmol) in dry 1,4-dioxane (10 mL) under nitrogen atmosphere. Then dimethylzinc solution 2M in toluene (0.75 mL, 1.51 mmol) was added dropwise. The mixture was stirred at 50° C. for 16 h. Water was added and extracted with EtOAc (×3). The combined organic layers were dried over MgSO4, filtered and concentrated in vacuo. The crude product was purified by flash column chromatography (silica; AcOEt in n-heptane from 0/100 to 30/70). The desired fractions were collected and the solvents evaporated in vacuo to intermediate C-59 as a white solid, 352 mg (86%).

### Preparation of Intermediate C-60

Accordingly, intermediate C-60 was prepared in the same way as intermediate C-9 starting from intermediate C-59 (352 mg, 0.95 mmol) to afford intermediate C-60 as a sticky yellow solid, 475 mg (quant.).

### Preparation of Intermediate C-61

Accordingly, intermediate C-61 was prepared in the same way as intermediate C-3 starting from intermediate C-60 (475 mg, 0.95 mmol) to afford intermediate C-61 as a white solid, 447 mg (quant.).

### Preparation of Compound 85

Accordingly, compound 85 was prepared in the same way as compound 1 starting from intermediate AI-3 (157.3 mg, 0.54 mmol) and intermediate C-61 (200 mg, 0.45 mmol) yielding 90 mg (35%) as a beige solid.

### Synthesis of Compound 86

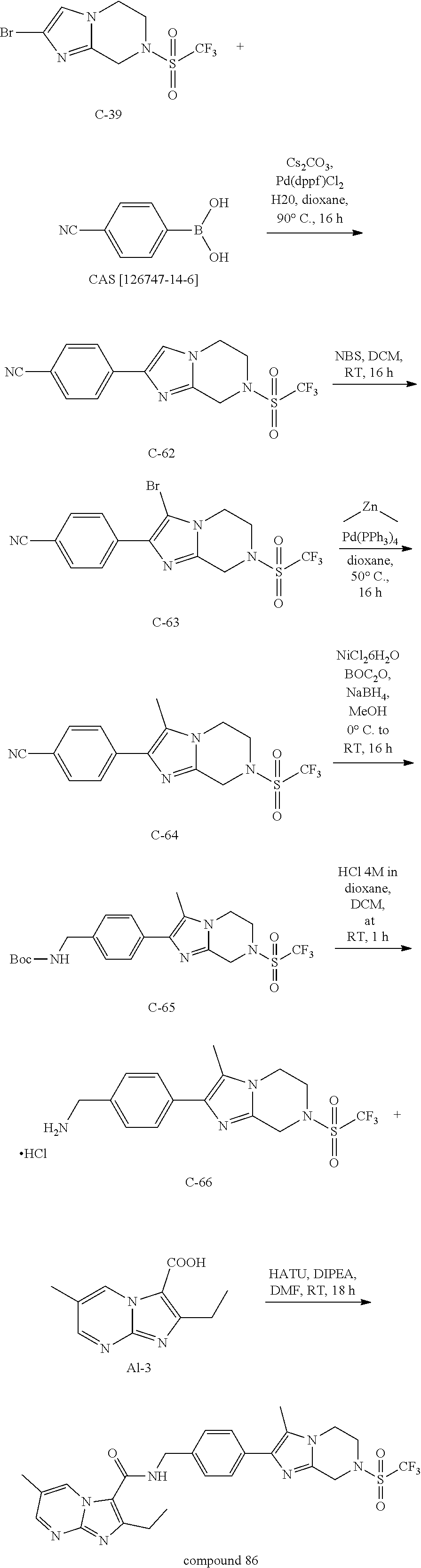

### Preparation of Intermediate C-62

Accordingly, intermediate C-62 was prepared in the same way as intermediate C-41 starting from intermediate C-39 (150 mg, 0.45 mmol) and 4-cyanophenylboronic acid (CAS [126747-14-6], 92 mg, 0.63 mmol) affording intermediate C-62 as a pale yellow solid, 107 mg (660%).

### Preparation of Intermediate C-63

N-Bromosuccinimide (54 mg, 0.3 mmol) was added to a solution of intermediate C-62 (107 mg, 0.3 mmol) in DCM (4 mL) in a round bottom flask at rt. The mixture was stirred at rt for 16 h. Water was added and the mixture was extracted with DCM. The organic layer was dried over MgSO4, filtered and concentrated in vacuo. The crude product was purified by flash column chromatography (silica; EtOAc in heptane 0/100 to 30/70). The desired fractions were collected and concentrated in vacuo to yield intermediate C-63 as a pale yellow solid, 114 mg (83%).

### Preparation of Intermediate C-64

Accordingly, intermediate C-64 was prepared in the same way as intermediate C-59 starting from intermediate C-63 (114 mg, 0.26 mmol) affording intermediate C-64 as a pale brown solid, 78 mg (80%).

### Preparation of Intermediate C-65

Accordingly, intermediate C-65 was prepared in the same way as intermediate C-9 starting from intermediate C-64 (78 mg, 0.21 mmol) to afford intermediate C-65 as a white solid, 89 mg (85%).

### Preparation of Intermediate C-66

Accordingly, intermediate C-66 was prepared in the same way as intermediate C-3 starting from intermediate C-65 (89 mg, 0.19 mmol) to afford intermediate C-66 as a pale yellow solid, 74 mg (84%).

### Preparation of Compound 86

Accordingly, compound 86 was prepared in the same way as compound 1 starting from intermediate AI-3 (73 mg, 0.25 mmol) and intermediate C-66 (74 mg, 0.17 mmol) yielding 30 mg (32%) as an off white solid.

### Synthesis of Compound 87

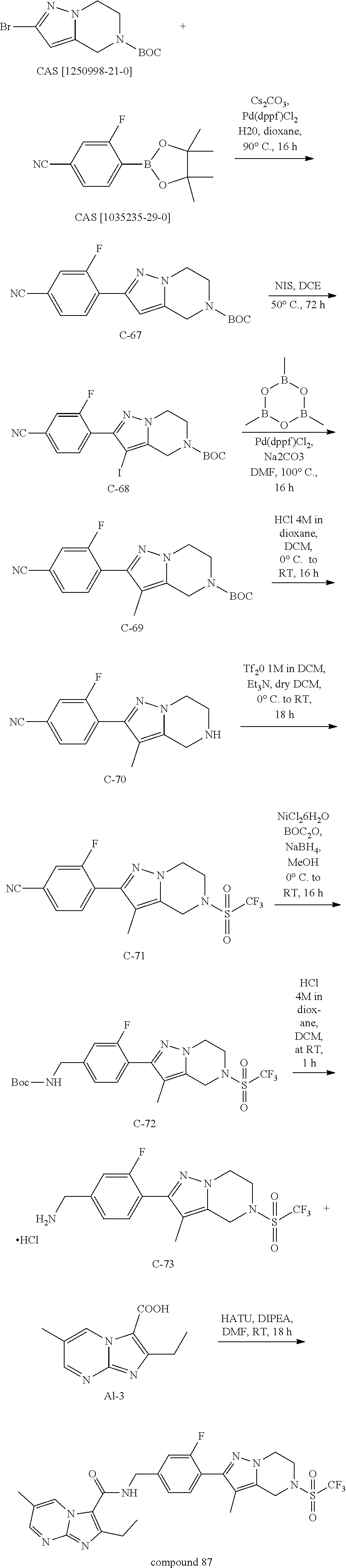

### Preparation of Intermediate C-67

Accordingly, intermediate C-67 was prepared in the same way as intermediate C-41 starting from tert-butyl 2-bromo-6,7-dihydro-4H-pyrazolo[1,5-a]pyrazine-5-carboxylate (CAS [1250998-21-0], 1.06 g, 3.49 mmol) and 4-cyano-2-fluorophenylboronic acid pinacol ester (CAS [1035235-29-0], 950 mg, 3.84 mmol) affording intermediate C-67 as a beige solid, 766 mg (58%).

### Preparation of Intermediate C-68

N-Iodosuccinimide [516-12-1] (755 mg, 3.36 mmol) was added to a stirred solution of intermediate C-67 (766 mg, 2.24 mmol) in DCM (22 mL) at rt. The mixture reaction was stirred at rt for 16 h. 1,2-Dichloroethane (22 mL) and N-Iodosuccinimide [516-12-1] (503 mg, 2.24 mmol) were added at rt and the reaction was stirred at 50° C. for 72 h. Aqueous saturated Na2S2O3 solution was added and extracted with DCM. The organic layer was dried over MgSO4, filtered and concentrated in vacuo to give a yellow oil. The crude product was purified by flash column chromatography (silica; EtOAc in heptane 0/100 to 30/70). The desired fractions were collected and concentrated in vacuo to afford intermediate C-68 as a white foam, 729 mg (63%).

### Preparation of Intermediate C-69

Pd(dppf)Cl2 [95464-05-4] (88 mg, 0.11 mmol) was added to a stirred mixture of intermediate C-68 (330 mg, 0.71 mmol), trimethylboroxine [823-96-1] (286 uL, 2.05 mmol) and sodium carbonate (308 mg, 2.91 mmol) in anhydrous DMF in a screw top vial and it was bubbled with nitrogen. Then the mixture was stirred at 100° C. for 16 h. Water was added and extracted with EtOAc (×3). The combined organic layers were dried over MgSO4, filtered and concentrated in vacuo to give a dark solid. The crude product was purified by flash column chromatography (silica; EtOAc in heptane 0/100 to 45/55). The desired fractions were collected and concentrated in vacuo to afford intermediate C-69 as a brown solid, 181 mg (68%).

### Preparation of Intermediate C-70

Accordingly, intermediate C-70 was prepared in the same way as intermediate C-3 starting from intermediate C-69 (324 mg, 0.91 mmol) to afford intermediate C-70 as a white solid, 280 mg (99%).

### Preparation of Intermediate C-71

Accordingly, intermediate C-71 was prepared in the same way as intermediate A-3 starting from intermediate C-70 (280 mg, 0.96 mmol) affording intermediate C-71 as a white solid, 177 mg (45%).

### Preparation of Intermediate C-72

Accordingly, intermediate C-72 was prepared in the same way as intermediate C-9 starting from intermediate C-71 (177 mg, 0.46 mmol) to afford intermediate C-72 as a brown solid, 144 mg (63%).

### Preparation of Intermediate C-73

Accordingly, intermediate C-73 was prepared in the same way as intermediate C-3 starting from intermediate C-72 (144 mg, 0.29 mmol) to afford intermediate C-73 as a white solid, 143 mg (99%).

### Preparation of Compound 87

Accordingly, compound 87 was prepared in the same way as compound 1 starting from intermediate AI-3 (139 mg, 0.47 mmol) and intermediate C-73 (143 mg, 0.31 mmol) yielding 95 mg (53%) as a beige solid.

### Synthesis of Compound 88

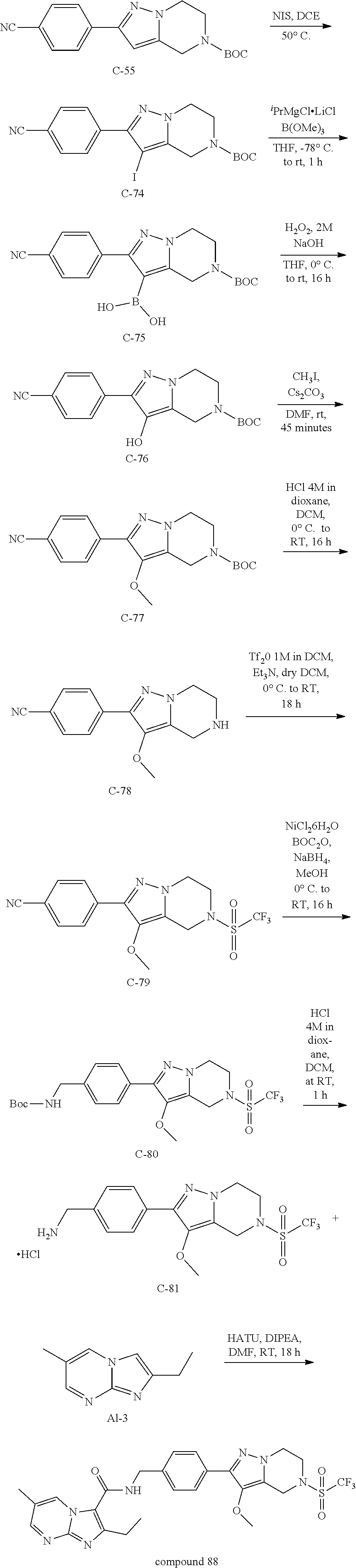

### Preparation of Intermediate C-74

Accordingly, intermediate C-74 was prepared in the same way as intermediate C-68 starting from intermediate C-55 (450 mg, 1.39 mmol) affording intermediate C-74 as a white solid, 344 mg (55%).

### Preparation of Intermediate C-75

Isopropylmagnesium chloride lithium chloride complex solution 1.3M (0.67 mL, 0.87 mmol) was added dropwise to a stirred solution of intermediate C-74 (325 mg, 0.72 mmol) in anhydrous THF (7 mL) at −78° C. under N2 atmosphere. The mixture was stirred at −78° C. for 5 minutes and then trimethyl borate [121-43-7] (0.23 mL, 2.06 mmol) was added dropwise. The mixture was stirred at −78° C. for 30 min. and at rt for 1h. The mixture was diluted with water and extracted with EtOAc. The organic layer was separated, dried over MgSO4, filtered and concentrated in vacuo to afford intermediate C-75 as a white foam, 0.28 g (52%).

### Preparation of Intermediate C-76

NaOH 2M aqueous solution (0.72 mL, 1.44 mmol) was added to a stirred solution of intermediate C-75 (266 mg, 0.72 mmol) and hydrogen peroxide 30% wt [7722-84-1] (0.15 mL, 1.45 mmol) in THF (7 mL) at 0° C. The mixture was stirred at rt for 16 h. The mixture was diluted with water and extracted with EtOAc. The organic layer was separated, dried over MgSO4, filtered and concentrated in vacuo to give a yellow solid. The crude product was purified by flash column chromatography (silica; EtOAc in heptane from 0/100 to 40/60). The desired fractions were collected and concentrated in vacuo to afford intermediate C-76 as a yellow solid, 55 mg (20%).

### Preparation of Intermediate C-77

Iodomethane [74-88-4] (0.015 mL, 0.24 mmol) was added to a stirred suspension of intermediate C-76 (55 mg, 0.16 mmol) and Cs2CO3 (105 mg, 0.32 mmol) in DMF (2 mL). The mixture was stirred at rt for 45 min. Water was added and extracted with EtOAc. The organic layer was dried over MgSO4, filtered and concentrated in vacuo to give a yellow oil. The crude product was purified by flash column chromatography (silica; EtOAc in heptane 0/100 to 40/60). The desired fractions were collected, concentrated in vacuo to yield intermediate C-77 as a yellow solid, 34 mg (56%).

### Preparation of Intermediate C-78

Accordingly, intermediate C-78 was prepared in the same way as intermediate C-3 starting from intermediate C-77 (78 mg, 0.22 mmol) to afford intermediate C-78 as a white solid, 62 mg (92%).

### Preparation of Intermediate C-79

Accordingly, intermediate C-79 was prepared in the same way as intermediate A-3 starting from intermediate C-78 (62 mg, 0.21 mmol) affording intermediate C-79 as a white solid, 63 mg (69%).

### Preparation of Intermediate C-80

Accordingly, intermediate C-80 was prepared in the same way as intermediate C-9 starting from intermediate C-79 (63 mg, 0.16 mmol) to afford intermediate C-80 as a beige solid, 73 mg (84%).

### Preparation of Intermediate C-81

Accordingly, intermediate C-81 was prepared in the same way as intermediate C-3 starting from intermediate C-80 (73 mg, 0.15 mmol) to afford intermediate C-81 as a white solid, 69 mg (99%).

### Preparation of Compound 88

Accordingly, compound 88 was prepared in the same way as compound 1 starting from intermediate AI-3 (74 mg, 0.25 mmol) and intermediate C-81 (69 mg, 0.15 mmol) yielding 28 mg (32%) as a white solid.

### 3. Characterizing Data Table

Various other compounds that are not specifically described above were also prepared in accordance with the methods described herein (as depicted below) and are also characterised in the table below:

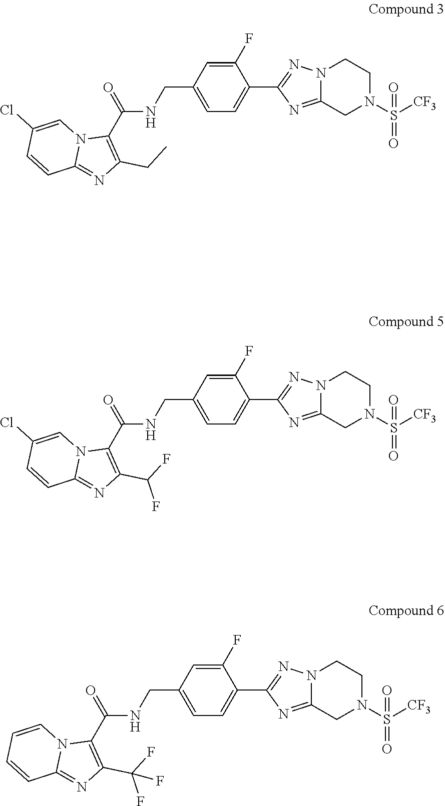 [SMILES: CCc1nc2ccc(Cl)cn2c1C(=O)NCc1ccc(-c2nc3n(n2)CCN(S(=O)(=O)C(F)(F)F)C3)c(F)c1 ; O=C(NCc1ccc(-c2nc3n(n2)CCN(S(=O)(=O)C(F)(F)F)C3)c(F)c1)c1c(C(F)F)nc2ccc(Cl)cn12 ; O=C(NCc1ccc(-c2nc3n(n2)CCN(S(=O)(=O)C(F)(F)F)C3)c(F)c1)c1c(C(F)(F)F)nc2ccccn12]

### Compounds 9-20

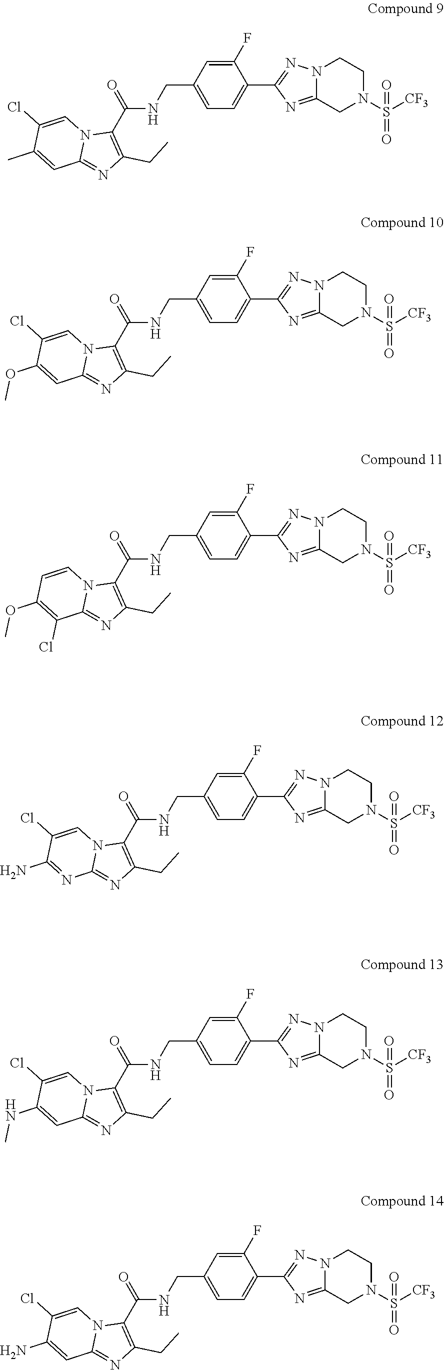 [SMILES: CCc1nc2cc(C)c(Cl)cn2c1C(=O)NCc1ccc(-c2nc3n(n2)CCN(S(=O)(=O)C(F)(F)F)C3)c(F)c1 ; CCc1nc2cc(OC)c(Cl)cn2c1C(=O)NCc1ccc(-c2nc3n(n2)CCN(S(=O)(=O)C(F)(F)F)C3)c(F)c1 ; CCc1nc2c(Cl)c(OC)ccn2c1C(=O)NCc1ccc(-c2nc3n(n2)CCN(S(=O)(=O)C(F)(F)F)C3)c(F)c1 ; CCc1nc2nc(N)c(Cl)cn2c1C(=O)NCc1ccc(-c2nc3n(n2)CCN(S(=O)(=O)C(F)(F)F)C3)c(F)c1 ; CCc1nc2cc(NC)c(Cl)cn2c1C(=O)NCc1ccc(-c2nc3n(n2)CCN(S(=O)(=O)C(F)(F)F)C3)c(F)c1 ; CCc1nc2cc(N)c(Cl)cn2c1C(=O)NCc1ccc(-c2nc3n(n2)CCN(S(=O)(=O)C(F)(F)F)C3)c(F)c1]

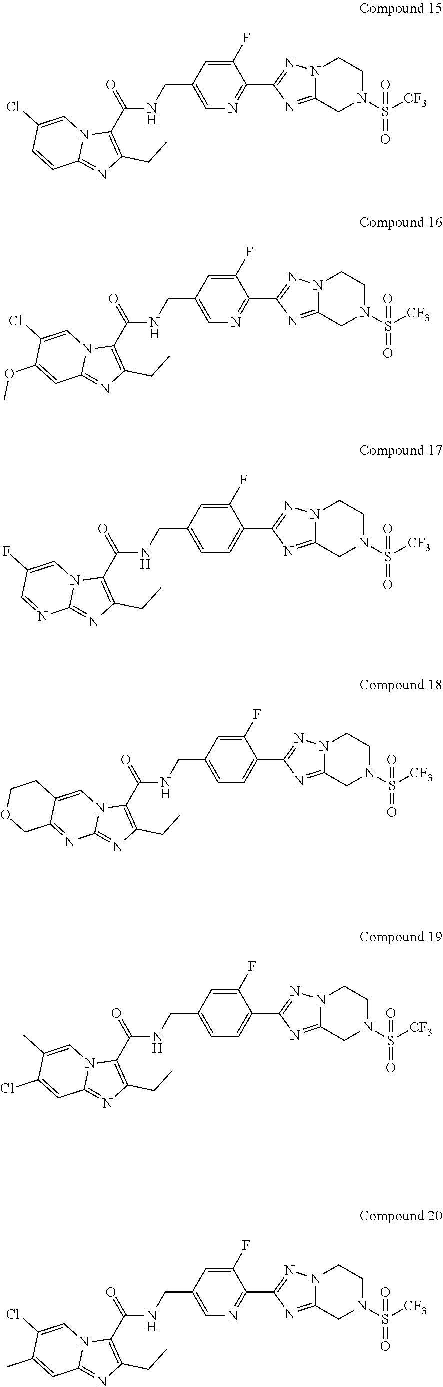 [SMILES: CCc1nc2ccc(Cl)cn2c1C(=O)NCc1cnc(-c2nc3n(n2)CCN(S(=O)(=O)C(F)(F)F)C3)c(F)c1 ; CCc1nc2cc(OC)c(Cl)cn2c1C(=O)NCc1cnc(-c2nc3n(n2)CCN(S(=O)(=O)C(F)(F)F)C3)c(F)c1 ; CCc1nc2ncc(F)cn2c1C(=O)NCc1ccc(-c2nc3n(n2)CCN(S(=O)(=O)C(F)(F)F)C3)c(F)c1 ; CCc1nc2nc3c(cn2c1C(=O)NCc1ccc(-c2nc4n(n2)CCN(S(=O)(=O)C(F)(F)F)C4)c(F)c1)CCOC3 ; CCc1nc2cc(Cl)c(C)cn2c1C(=O)NCc1ccc(-c2nc3n(n2)CCN(S(=O)(=O)C(F)(F)F)C3)c(F)c1 ; CCc1nc2cc(C)c(Cl)cn2c1C(=O)NCc1cnc(-c2nc3n(n2)CCN(S(=O)(=O)C(F)(F)F)C3)c(F)c1]

### Compound 25-27, 32, 35, 37

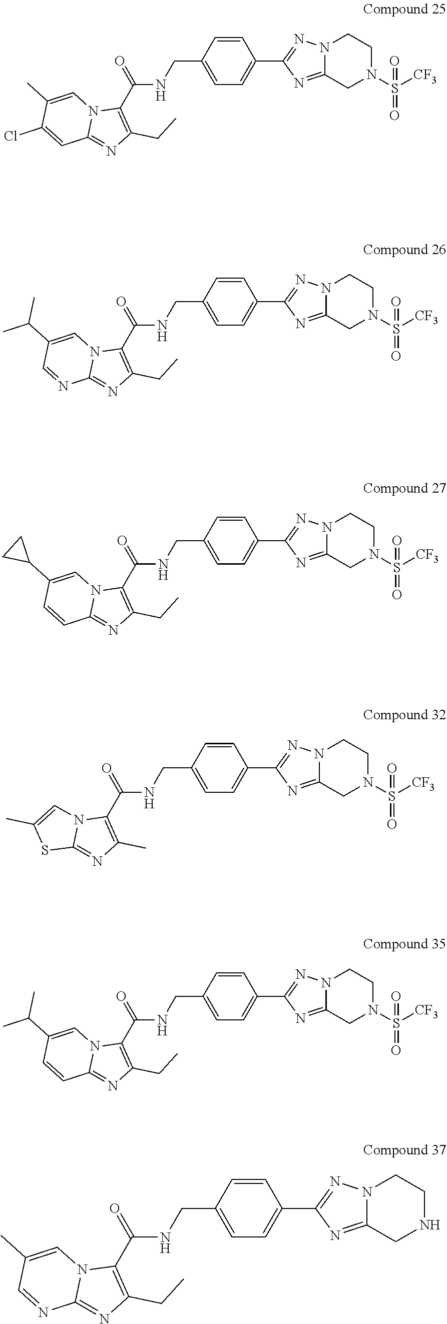 [SMILES: CCc1nc2cc(Cl)c(C)cn2c1C(=O)NCc1ccc(-c2nc3n(n2)CCN(S(=O)(=O)C(F)(F)F)C3)cc1 ; CCc1nc2ncc(C(C)C)cn2c1C(=O)NCc1ccc(-c2nc3n(n2)CCN(S(=O)(=O)C(F)(F)F)C3)cc1 ; CCc1nc2ccc(C3CC3)cn2c1C(=O)NCc1ccc(-c2nc3n(n2)CCN(S(=O)(=O)C(F)(F)F)C3)cc1 ; Cc1cn2c(C(=O)NCc3ccc(-c4nc5n(n4)CCN(S(=O)(=O)C(F)(F)F)C5)cc3)c(C)nc2s1 ; CCc1nc2ccc(C(C)C)cn2c1C(=O)NCc1ccc(-c2nc3n(n2)CCN(S(=O)(=O)C(F)(F)F)C3)cc1 ; CCc1nc2ncc(C)cn2c1C(=O)NCc1ccc(-c2nc3n(n2)CCNC3)cc1]

### Compounds 38-41, 43, 46

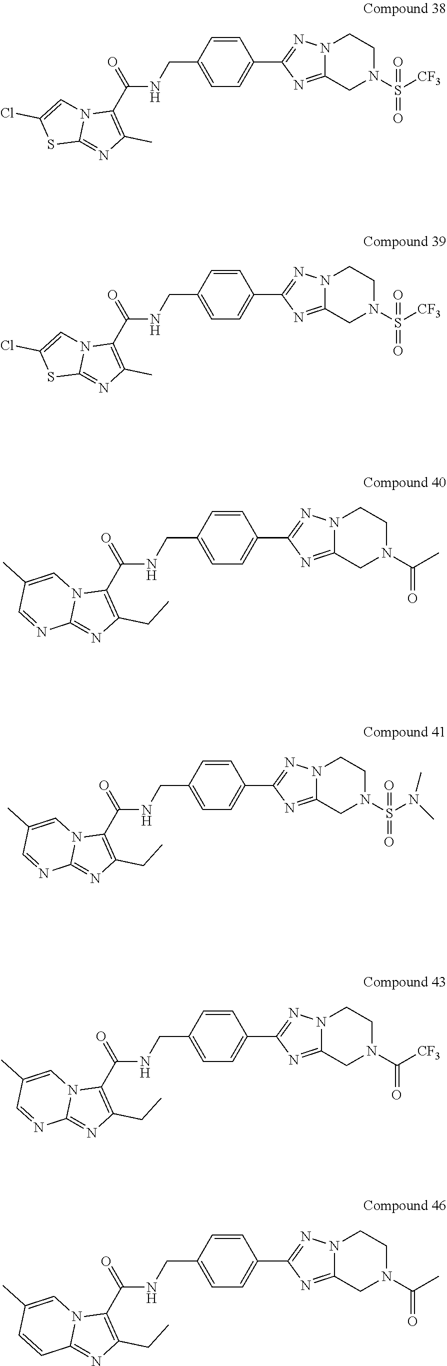

### Compounds 49,50,52-54, 57

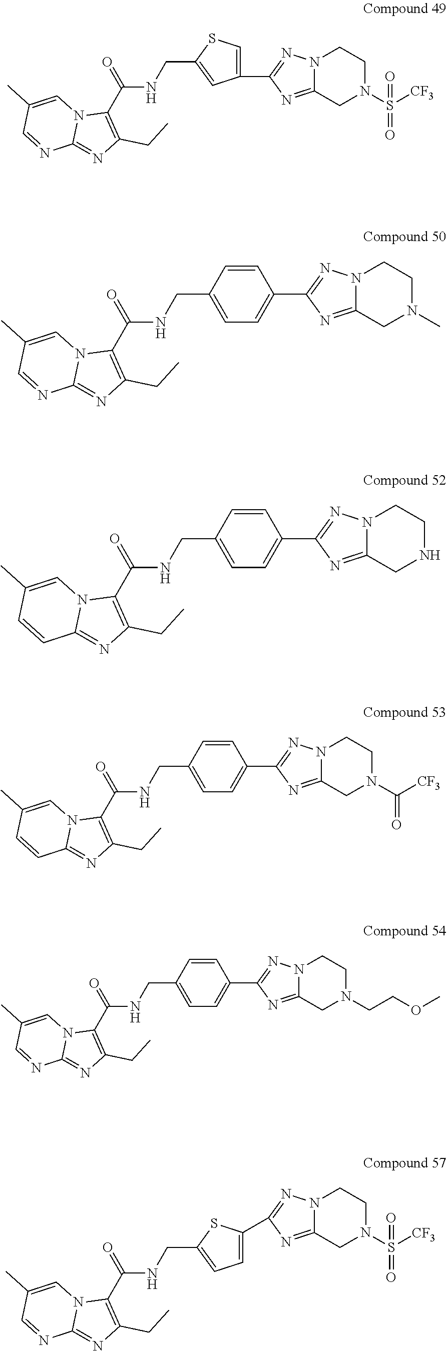

### Compounds 58-62

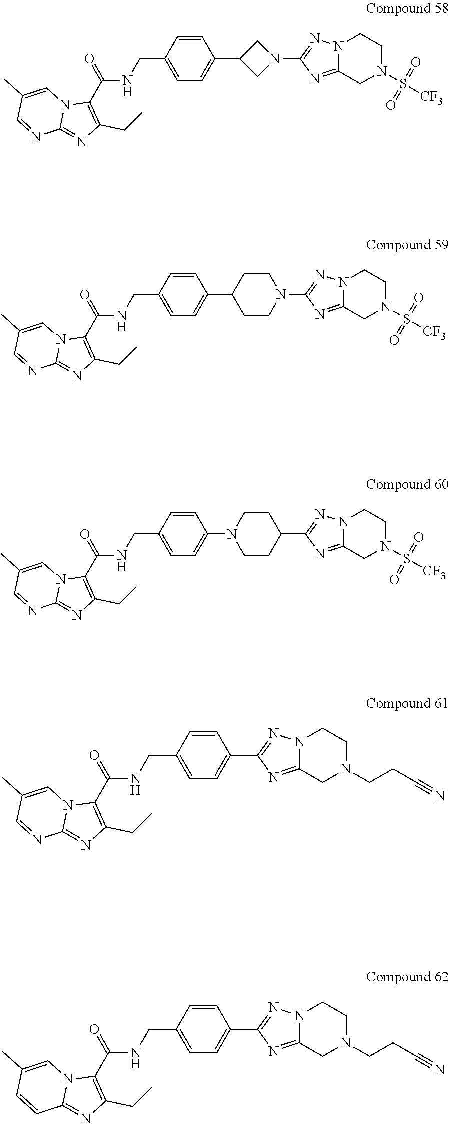 [SMILES: CCc1nc2ncc(C)cn2c1C(=O)NCc1ccc(C2CN(c3nc4n(n3)CCN(S(=O)(=O)C(F)(F)F)C4)C2)cc1 ; CCc1nc2ncc(C)cn2c1C(=O)NCc1ccc(C2CCN(c3nc4n(n3)CCN(S(=O)(=O)C(F)(F)F)C4)CC2)cc1 ; CCc1nc2ncc(C)cn2c1C(=O)NCc1ccc(N2CCC(c3nc4n(n3)CCN(S(=O)(=O)C(F)(F)F)C4)CC2)cc1 ; CCc1nc2ncc(C)cn2c1C(=O)NCc1ccc(-c2nc3n(n2)CCN(CCC#N)C3)cc1 ; CCc1nc2ccc(C)cn2c1C(=O)NCc1ccc(-c2nc3n(n2)CCN(CCC#N)C3)cc1]

### Compounds 63 and 64

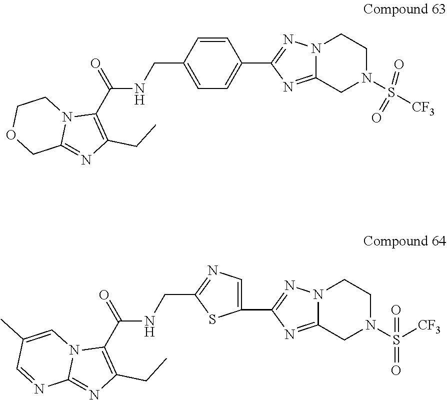 [SMILES: CCc1nc2n(c1C(=O)NCc1ccc(-c3nc4n(n3)CCN(S(=O)(=O)C(F)(F)F)C4)cc1)CCOC2 ; CCc1nc2ncc(C)cn2c1C(=O)NCc1ncc(-c2nc3n(n2)CCN(S(=O)(=O)C(F)(F)F)C3)s1]

The following compound was also prepared in accordance with the procedures described herein:

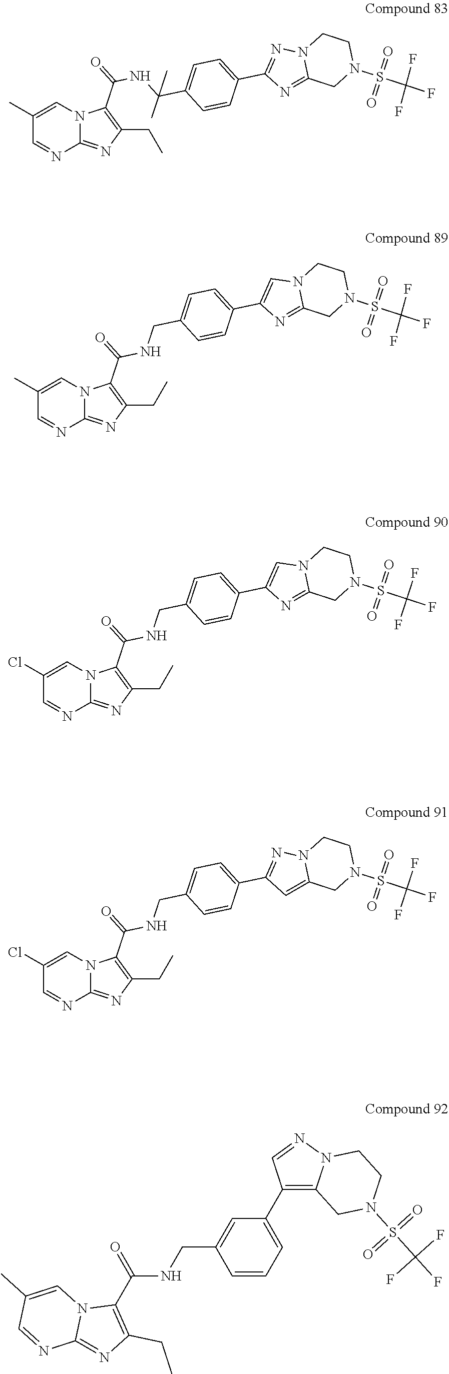 [SMILES: CCc1nc2ncc(C)cn2c1C(=O)NC(C)(C)c1ccc(-c2nc3n(n2)CCN(S(=O)(=O)C(F)(F)F)C3)cc1 ; CCc1nc2ncc(C)cn2c1C(=O)NCc1ccc(-c2cn3c(n2)CN(S(=O)(=O)C(F)(F)F)CC3)cc1 ; CCc1nc2ncc(Cl)cn2c1C(=O)NCc1ccc(-c2cn3c(n2)CN(S(=O)(=O)C(F)(F)F)CC3)cc1 ; CCc1nc2ncc(Cl)cn2c1C(=O)NCc1ccc(-c2cc3n(n2)CCN(S(=O)(=O)C(F)(F)F)C3)cc1 ; CCc1nc2ncc(C)cn2c1C(=O)NCc1cccc(-c2cnn3c2CN(S(=O)(=O)C(F)(F)F)CC3)c1]

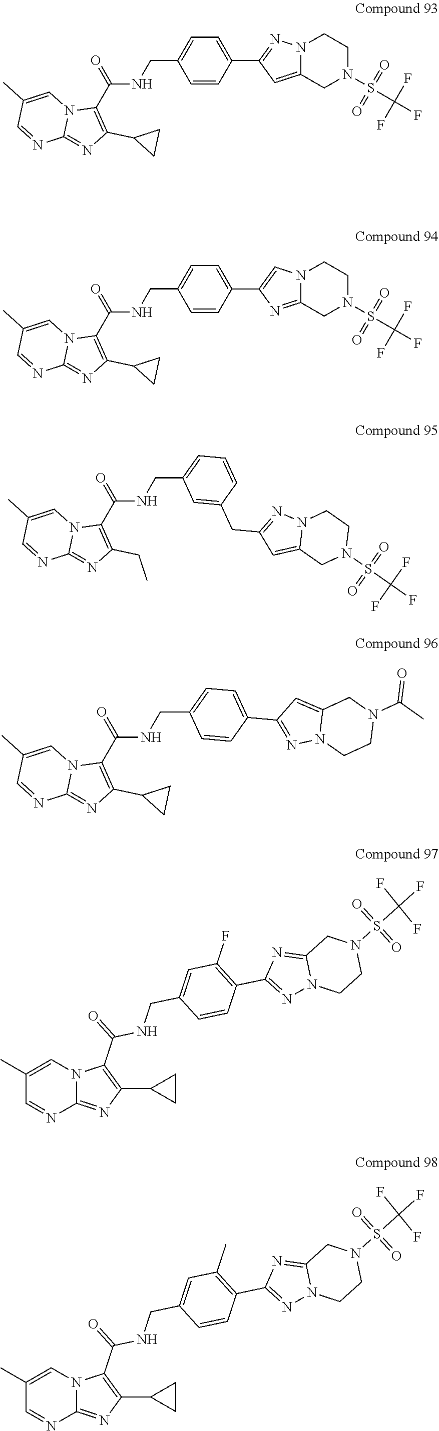

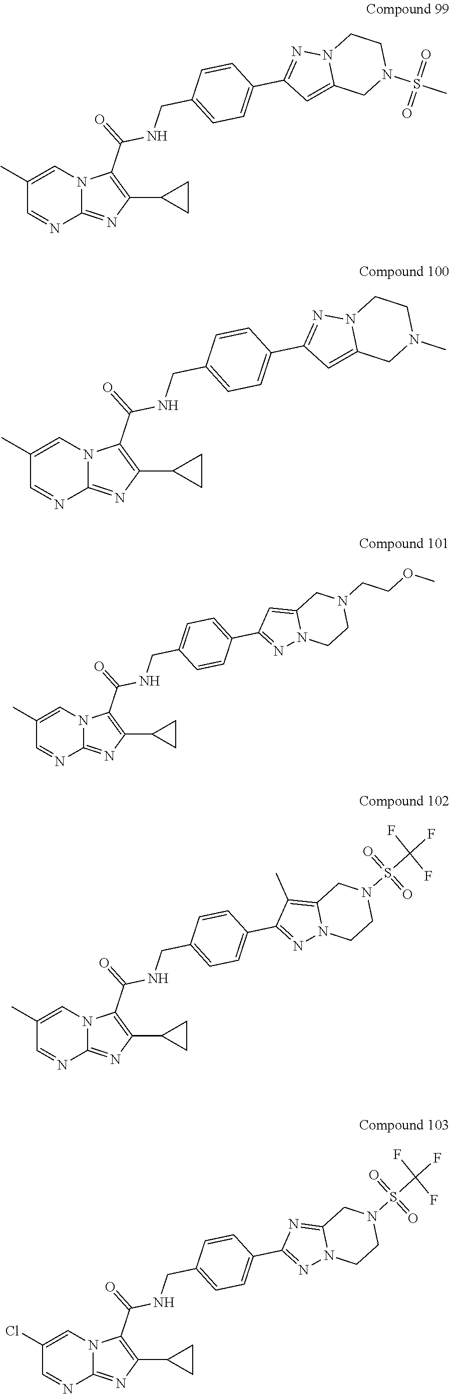

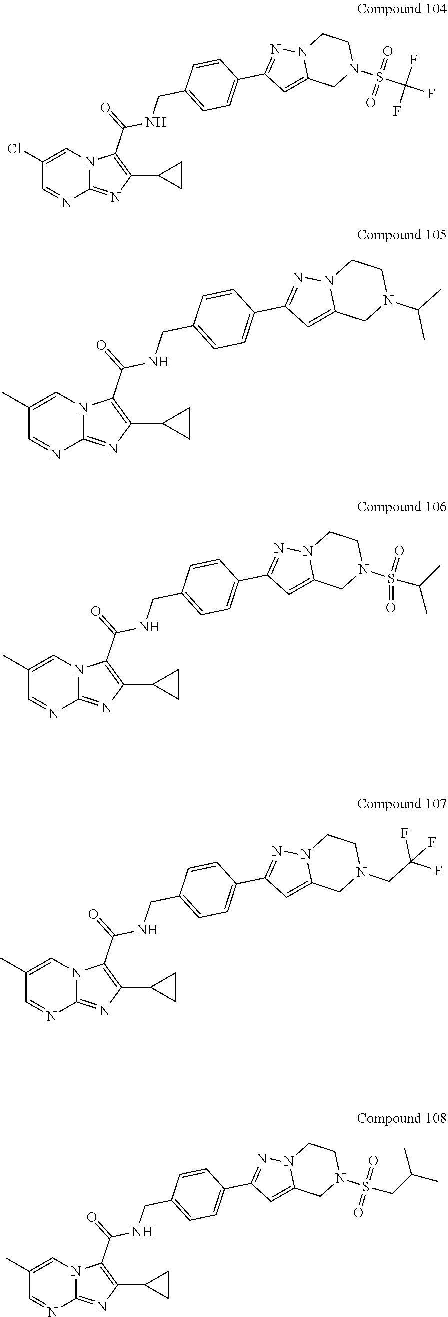

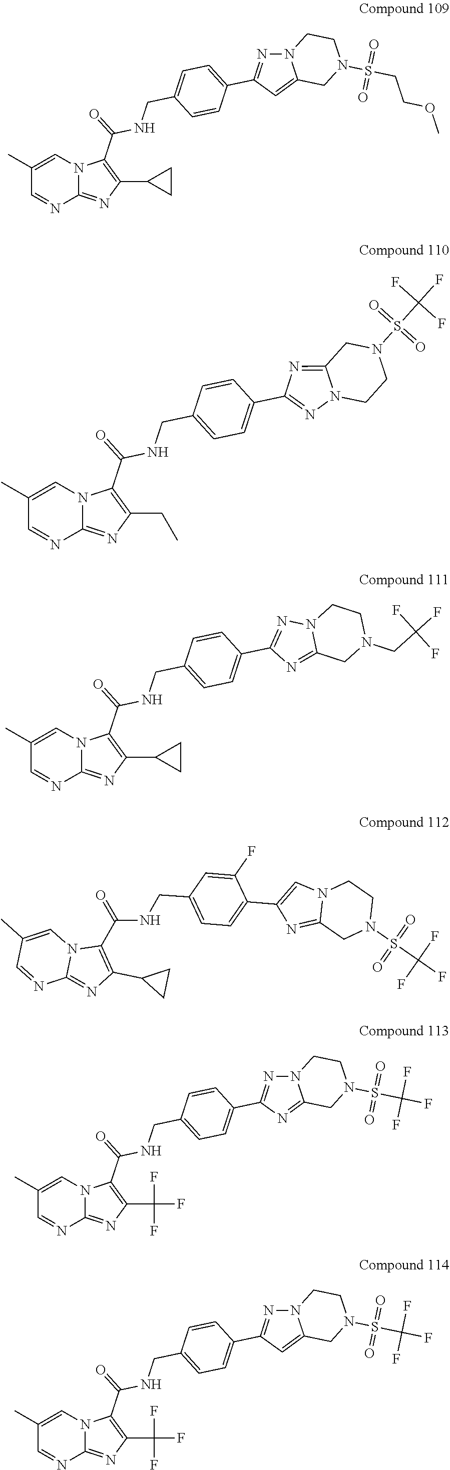

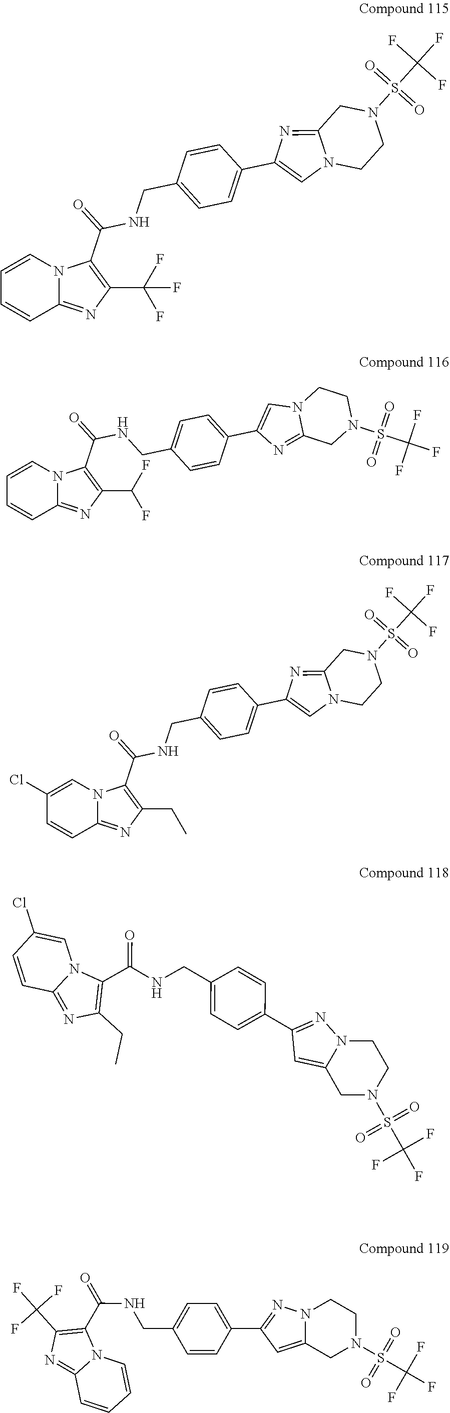 [SMILES: O=C(NCc1ccc(-c2cn3c(n2)CN(S(=O)(=O)C(F)(F)F)CC3)cc1)c1c(C(F)(F)F)nc2ccccn12 ; O=C(NCc1ccc(-c2cn3c(n2)CN(S(=O)(=O)C(F)(F)F)CC3)cc1)c1c(C(F)F)nc2ccccn12 ; CCc1nc2ccc(Cl)cn2c1C(=O)NCc1ccc(-c2cn3c(n2)CN(S(=O)(=O)C(F)(F)F)CC3)cc1 ; CCc1nc2ccc(Cl)cn2c1C(=O)NCc1ccc(-c2cc3n(n2)CCN(S(=O)(=O)C(F)(F)F)C3)cc1 ; O=C(NCc1ccc(-c2cc3n(n2)CCN(S(=O)(=O)C(F)(F)F)C3)cc1)c1c(C(F)(F)F)nc2ccccn12]

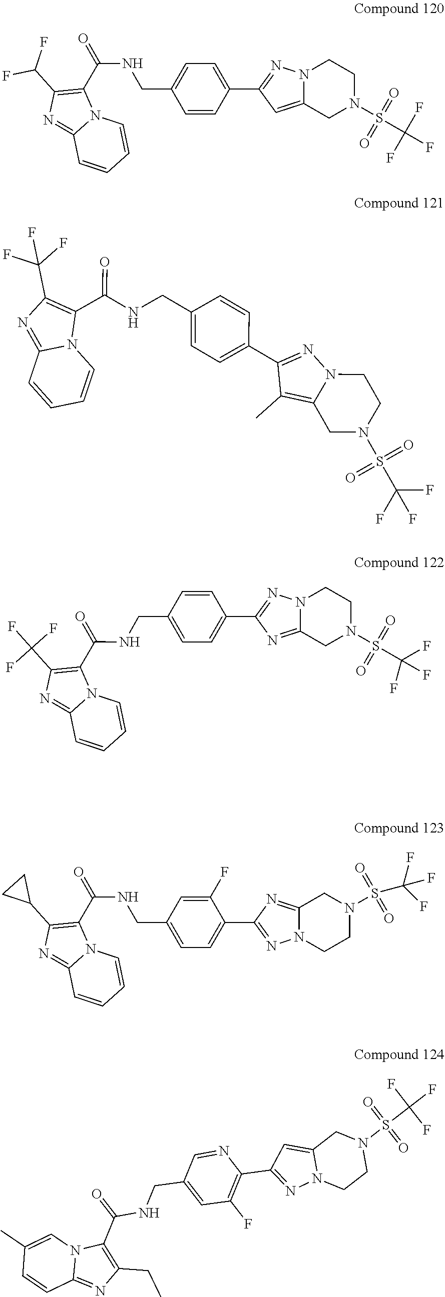 [SMILES: O=C(NCc1ccc(-c2cc3n(n2)CCN(S(=O)(=O)C(F)(F)F)C3)cc1)c1c(C(F)F)nc2ccccn12 ; Cc1c(-c2ccc(CNC(=O)c3c(C(F)(F)F)nc4ccccn34)cc2)nn2c1CN(S(=O)(=O)C(F)(F)F)CC2 ; O=C(NCc1ccc(-c2nc3n(n2)CCN(S(=O)(=O)C(F)(F)F)C3)cc1)c1c(C(F)(F)F)nc2ccccn12 ; O=C(NCc1ccc(-c2nc3n(n2)CCN(S(=O)(=O)C(F)(F)F)C3)c(F)c1)c1c(C2CC2)nc2ccccn12 ; CCc1nc2ccc(C)cn2c1C(=O)NCc1cnc(-c2cc3n(n2)CCN(S(=O)(=O)C(F)(F)F)C3)c(F)c1]

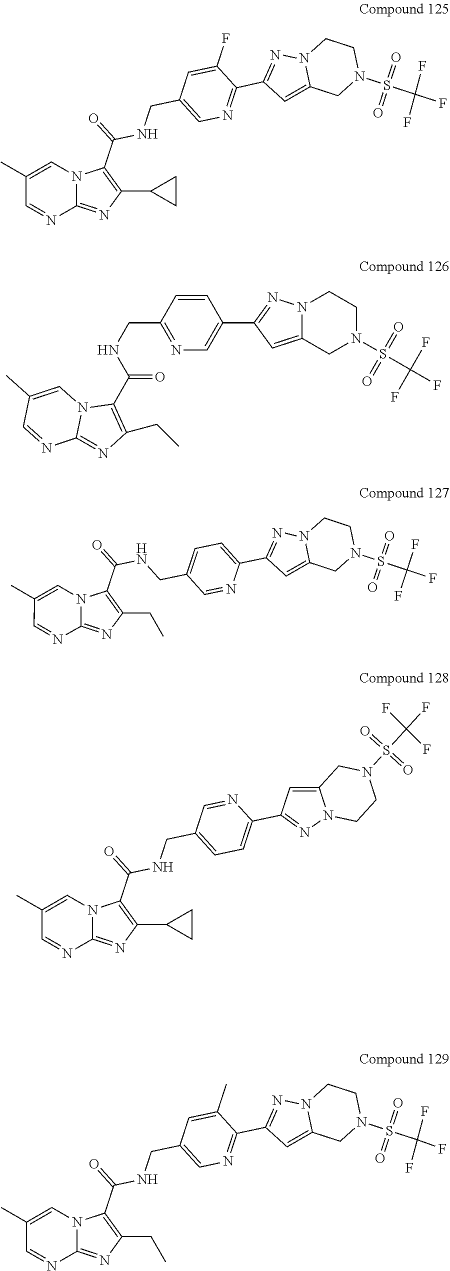 [SMILES: Cc1cnc2nc(C3CC3)c(C(=O)NCc3cnc(-c4cc5n(n4)CCN(S(=O)(=O)C(F)(F)F)C5)c(F)c3)n2c1 ; CCc1nc2ncc(C)cn2c1C(=O)NCc1ccc(-c2cc3n(n2)CCN(S(=O)(=O)C(F)(F)F)C3)cn1 ; CCc1nc2ncc(C)cn2c1C(=O)NCc1ccc(-c2cc3n(n2)CCN(S(=O)(=O)C(F)(F)F)C3)nc1 ; Cc1cnc2nc(C3CC3)c(C(=O)NCc3ccc(-c4cc5n(n4)CCN(S(=O)(=O)C(F)(F)F)C5)nc3)n2c1 ; CCc1nc2ncc(C)cn2c1C(=O)NCc1cnc(-c2cc3n(n2)CCN(S(=O)(=O)C(F)(F)F)C3)c(C)c1]

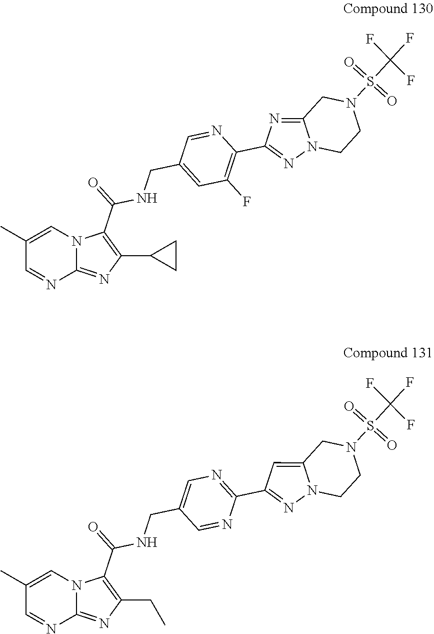 [SMILES: Cc1cnc2nc(C3CC3)c(C(=O)NCc3cnc(-c4nc5n(n4)CCN(S(=O)(=O)C(F)(F)F)C5)c(F)c3)n2c1 ; CCc1nc2ncc(C)cn2c1C(=O)NCc1cnc(-c2cc3n(n2)CCN(S(=O)(=O)C(F)(F)F)C3)nc1]

| | LC-MS | | | | | | |
|---|---|---|---|---|---|---|---|
| cpd | | % | | BPM | BPM | | |
| nbr | RT | UV | MW | 1 | 2 | Method | mp |

| | | | | | | |
|---|---|---|---|---|---|---|
| 1 | 9.4 | 99.69 | 567.1 | 568.1 | A | 202.3° C. (DSC |
| | | | | | | Mettler |
| | | | | | | Toledo |
| | | | | | | (5° C./min) |
| 2 | 9.7 | 99.66 | 568.1 | 569.1 | A | 216.0° C. (DSC |
| | | | | | | Mettler |
| | | | | | | Toledo |
| | | | | | | (5° C./min) |
| 3 | 9.4 | 99.66 | 585.1 | 586.1 | A | 221.6° C. (DSC |
| | | | | | | 1 Mettler |
| | | | | | | Toledo |
| | | | | | | (5° C./min) |
| 4 | 9.6 | 99.37 | 586.1 | 587.1 | A | 229.8° C. (DSC |
| | | | | | | 1 Mettler |
| | | | | | | Toledo |
| | | | | | | (5° C./min) |
| 5 | 3.51 | 97 | 607 | 608 | B | 201.5° C. |
| | | | | | | (Mettler |
| | | | | | | Toledo |
| | | | | | | MP50) |
| 6 | 3.32 | 99 | 591.1 | 592.1 | B | 243.9° C. |
| | | | | | | (Mettler |
| | | | | | | Toledo |
| | | | | | | MP50) |
| 7 | 2.802 | 97 | 566.1 | 567.1 | B | 243.4° C. |
| | | | | | | (Mettler |
| | | | | | | Toledo |
| | | | | | | MP50) |
| 8 | 3.309 | 99 | 600.1 | 601.1 | B | 234.9° C. |
| | | | | | | (Mettler |
| | | | | | | Toledo |
| | | | | | | MP50) |
| 9 | 3.043 | 99 | 599.1 | 600.1 | B | 248.3° C. |
| | | | | | | (Mettler |
| | | | | | | Toledo |
| | | | | | | MP50) |
| 10 | 2.82 | 99 | 615.1 | 616 | B | 270.0° C. |
| | | | | | | (Mettler |
| | | | | | | Toledo |
| | | | | | | MP50) |
| 11 | 2.801 | 99 | 615.1 | 616.1 | B | 233.4° C. |
| | | | | | | (Mettler |
| | | | | | | Toledo |
| | | | | | | MP50) |
| 12 | 3.2 | 97 | 601.1 | 602.3 | B | 273.3° C. |
| | | | | | | (Mettler |
| | | | | | | Toledo |
| | | | | | | MP50) |
| 13 | 2.64 | 99 | 614.1 | 615 | B | 230.0° C. |
| | | | | | | (Mettler |
| | | | | | | Toledo |
| | | | | | | MP50) |
| 14 | 2.652 | 99 | 600.1 | 601 | B | 263.4° C. |
| | | | | | | (Mettler |
| | | | | | | Toledo |
| | | | | | | MP50) |
| 15 | 2.813 | 97 | 586.1 | 586.9 | B | |
| 16 | 2.572 | 99 | 616.1 | 617 | B | 235.0° C. |
| | | | | | | (Metter |
| | | | | | | Toledo |
| | | | | | | MP50) |
| 17 | 3.09 | 99 | 570.1 | 571 | B | 263.4° C. |
| | | | | | | (Mettler |
| | | | | | | Toledo |
| | | | | | | MP50) |
| 18 | 2.78 | 98 | 608.1 | 609.1 | B | 243.4° C. |
| | | | | | | (Mettler |
| | | | | | | Toledo |
| | | | | | | MP50) |
| 19 | 3.126 | 99 | 599.1 | 600 | B | 238.4° C. |
| | | | | | | (Mettler |
| | | | | | | Toledo |
| | | | | | | MP50) |
| 20 | 2.717 | 95 | 600.1 | 601.1 | B | |
| 21 | 3.688 | 98 | 585 | 586.1 | B | 208.3° C. |
| | | | | | | (Metter |
| | | | | | | Toledo |
| | | | | | | (MP50)) |
| 22 | 2.902 | 99 | 550.1 | 552.1 | B | 212.15° C. |
| | | | | | | (Metter |
| | | | | | | Toledo |
| | | | | | | (MP50)) |
| 23 | 3.3 | 99 | 561.2 | 562.2 | B | 216.6° C. |
| | | | | | | (Metter |
| | | | | | | Toledo |
| | | | | | | MP50) |
| 24 | 2.548 | 99 | 547.1 | 548.2 | B | 184.9° C. |
| | | | | | | (Metter |
| | | | | | | Toledo |
| | | | | | | (MP50)) |
| 25 | 3.114 | 99 | 581.1 | 582.1 | B | 236.7° C. |
| | | | | | | (Mettler |
| | | | | | | Toledo |
| | | | | | | MP50) |
| 26 | 3.225 | 96 | 576.2 | 577.2 | B | 198.2° C. |
| | | | | | | (Mettler |
| | | | | | | Toledo |
| | | | | | | MP50) |
| 27 | 2.76 | 95 | 573.2 | 574.2 | B | 174.8° C. |
| | | | | | | (Mettler |
| | | | | | | Toledo |
| | | | | | | MP50) |
| 28 | 3.124 | 99 | 574.2 | 575.2 | B | 194.9° C. |
| | | | | | | (Mettler |
| | | | | | | Toledo |
| | | | | | | MP50) |
| 29 | 2.716 | 99 | 534.1 | 535.1 | B | 241.6° C. |
| | | | | | | (Mettler |
| | | | | | | Toledo |
| | | | | | | MP50) |
| 30 | 2.71 | 97 | 559.2 | 560.2 | B | 196.6° C. |
| | | | | | | (Mettler |
| | | | | | | Toledo |
| | | | | | | MP50) |
| 31 | 2.397 | 99 | 537.2 | 538.2 | B | 164.8° C. |
| | | | | | | (Metter |
| | | | | | | Toledo |
| | | | | | | (MP50)) |
| 32 | 3.064 | 97 | 539.1 | 540.1 | B | 230.0° C. |
| | | | | | | (Mettler |
| | | | | | | Toledo |
| | | | | | | MP50) |
| 33 | 2.722 | 99 | 562.2 | 563.2 | B | 240.0° C. |
| | | | | | | (Mettler |
| | | | | | | Toledo |
| | | | | | | MP50) |
| 34 | 2.468 | 99 | 533.1 | 534.1 | B | 238.4° C. |
| | | | | | | (Metter |
| | | | | | | Toledo |
| | | | | | | (MP50)) |
| 35 | 2.853 | 99 | 575.2 | 576.2 | B | 181.5° C. |
| | | | | | | (Metter |
| | | | | | | Toledo |
| | | | | | | MP50) |
| 36 | 3.261 | 99 | 553.1 | 554.3 | B | 214.9° C. |
| | | | | | | (Metter |
| | | | | | | Toledo |
| | | | | | | (MP50)) |
| 37 | 1.446 | 99 | 416.2 | 417.2 | B | 256.7° C. |
| | | | | | | (Mettler |
| | | | | | | Toledo |
| | | | | | | MP50) |
| 38 | 3.478 | 99 | 559 | 560.1 | B | 253° C. |
| | | | | | | (Mettler |
| | | | | | | Toledo |
| | | | | | | MP50) |
| 39 | 3.69 | 99 | 573 | 574.1 | B | 182° C. |
| | | | | | | (Mettler |
| | | | | | | Toledo |
| | | | | | | MP50) |
| 40 | 1.903 | 99 | 458.2 | 459.2 | B | 240.0° C. |
| | | | | | | (Mettler |
| | | | | | | Toledo |
| | | | | | | MP50) |
| 41 | 2.335 | 95 | 523.2 | 524.3 | B | 216.6° C. |
| | | | | | | (Mettler |
| | | | | | | Toledo |
| | | | | | | MP50) |
| 42 | 3.081 | 99 | 560.1 | 561.2 | B | 232° C. |
| | | | | | | (Mettler |
| | | | | | | Toledo |
| | | | | | | MP50) |
| 43 | 2.471 | 99 | 512.2 | 513.2 | B | 245.1° C. |
| | | | | | | (Mettler |
| | | | | | | Toledo |
| | | | | | | MP50) |
| 44 | 3.104 | 99 | 547.2 | 548.2 | B | 235.0° C. |
| | | | | | | (Mettler |
| | | | | | | Toledo |
| | | | | | | MP50) |
| 45 | 2.788 | 99 | 574.2 | 575.2 | B | 246.7° C. |
| | | | | | | (Mettler |
| | | | | | | Toledo |
| | | | | | | MP50) |
| 46 | 1.81 | 99 | 457.2 | 458.2 | B | 209.9° C. |
| | | | | | | (Mettler |
| | | | | | | Toledo |
| | | | | | | MP50) |
| 47 | 2.523 | 99 | 549.2 | | B | 181.5 Â ° C. ° C. |
| | | | | | | (Mettler |
| | | | | | | Toledo |
| | | | | | | MP50) |
| 48 | 2.324 | 99 | 548.2 | 549.3 | B | 205.1° C. |
| | | | | | | (Mettler |
| | | | | | | Toledo |
| | | | | | | MP50) |
| 49 | 2.324 | 99 | 548.2 | 549.3 | B | 205.1° C. |
| | | | | | | (Mettler |
| | | | | | | Toledo |
| | | | | | | MP50) |
| 50 | 1.552 | 99 | 430.2 | 431.2 | B | 241.6° C. |
| | | | | | | (Mettler |
| | | | | | | Toledo |
| | | | | | | MP50) |
| 51 | 2.167 | 99 | 522.2 | 523.2 | B | 219.9° C. |
| | | | | | | (Mettler |
| | | | | | | Toledo |
| | | | | | | MP50) |
| 52 | 1.37 | 98 | 415.2 | 416.2 | B | 291.9° C. |
| | | | | | | (Mettler |
| | | | | | | Toledo |
| | | | | | | MP50) |
| 53 | 2.263 | 98 | 511.2 | 512.2 | B | 228.5° C. |
| | | | | | | (Metter |
| | | | | | | Toledo |
| | | | | | | MP50) |
| 54 | 1.832 | 99 | 474.3 | 475.3 | B | 116.4° C. |
| | | | | | | (Mettler |
| | | | | | | Toledo |
| | | | | | | MP50) |
| 55 | 1.767 | 99 | 473.2 | 474.3 | B | 198.2° C. |
| | | | | | | (Mettler |
| | | | | | | Toledo |
| | | | | | | MP50) |
| 56 | 1.502 | 99 | 429.2 | 430.2 | B | 218.3° C. |
| | | | | | | (Mettler |
| | | | | | | Toledo |
| | | | | | | MP50) |
| 57 | 2.813 | 99 | 554.1 | 555.1 | B | 223.2° C. |
| | | | | | | (Mettler |
| | | | | | | Toledo |
| | | | | | | MP50) |
| 58 | 2.815 | 99 | 603.2 | 604.3 | B | 158° C. |
| | | | | | | (Mettler |
| | | | | | | Toledo |
| | | | | | | MP50) |
| 59 | 3.193 | 98 | 631.2 | 632.4 | B | 159.8° C. |
| | | | | | | (Mettler |
| | | | | | | Toledo |
| | | | | | | MP50) |
| 60 | 2.308 | 97 | 631.2 | 632.2 | B | 189.9° C. |
| | | | | | | (Mettler |
| | | | | | | Toledo |
| | | | | | | MP50) |
| 61 | 2.072 | 99 | 469.2 | 471.4 | B | 251.7° C. |
| | | | | | | (Mettler |
| | | | | | | Toledo |
| | | | | | | MP50) |
| 62 | 1.953 | 99 | 468.2 | 469.2 | B | 218.2° C. |
| | | | | | | (Mettler |
| | | | | | | Toledo |
| | | | | | | MP50) |
| 63 | 2.369 | 99 | 539.1 | 540.3 | B | 221.6° C. |
| | | | | | | (Mettler |
| | | | | | | Toledo |
| | | | | | | MP50) |
| 64 | 2.64 | 97 | 555.1 | 556.1 | B | 226.7° C. |
| | | | | | | (Mettler |
| | | | | | | Toledo |
| | | | | | | MP50) |
| 65 | 2.149 | 95 | 552.6 | 553.2 | B | 193.2 |
| | | | | | | (Mettler |
| | | | | | | Toledo |
| | | | | | | MP50) |
| 66 | 2.825 | 97 | 562.6 | 563.2 | B | 168.0 |
| | | | | | | (Mettler |
| | | | | | | Toledo |
| | | | | | | MP50) |
| 67 | 2.853 | 98 | 548.5 | 549.1 | B | 116.2 |
| | | | | | | (Mettler |
| | | | | | | Toledo |
| | | | | | | MP50) |
| 68 | 2.919 | 99 | 574.6 | 575.1 | B | 164.7 |
| | | | | | | (Mettler |
| | | | | | | Toledo |
| | | | | | | MP50) |
| 69 | 2.696 | 97 | 617.6 | 618.2 | B | 196.6 |
| | | | | | | (Mettler |
| | | | | | | Toledo |
| | | | | | | MP50) |
| 70 | 2.885 | 99 | 632.7 | 633.2 | B | 211.5 |
| | | | | | | (Mettler |
| | | | | | | Toledo |
| | | | | | | MP50) |
| 71 | 2.731 | 98 | 562.6 | 563.2 | B | 96.2 |
| | | | | | | (Mettler |
| | | | | | | Toledo |
| | | | | | | MP50) |
| 72 | 2.385 | 99 | 539.5 | 540.1 | B | 208.2 |
| | | | | | | (Mettler |
| | | | | | | Toledo |
| | | | | | | MP50) |
| 73 | 2.626 | 99 | 555.6 | 556.1 | B | N/A |
| 74 | 2.577 | 99 | 555.5 | 556.0 | B | 153.1 |
| | | | | | | (Mettler |
| | | | | | | Toledo |
| | | | | | | MP50) |
| 75 | 2.551 | 99 | 555.6 | 556.1 | B | 229.9 |
| | | | | | | (Mettler |
| | | | | | | Toledo |
| | | | | | | MP50) |
| 76 | 2.528 | 99 | 556.5 | 557.1 | B | 248.4 |
| | | | | | | (Mettler |
| | | | | | | Toledo |
| | | | | | | MP50) |
| 77 | 2.869 | 97 | 587.9 | 588.0 | B | N/A |
| 78 | 2.237 | 99 | 601.9 | 602.1 | B | 237.2 |
| | | | | | | (Mettler |
| | | | | | | Toledo |
| | | | | | | MP50) |
| 79 | 2.252 | 95 | 546.6 | 547.2 | B | 176.4 |
| | | | | | | (Mettler |
| | | | | | | Toledo |
| | | | | | | MP50) |
| 80 | 2.879 | 98 | 565.5 | 565.8 | B | 220.0 |
| | | | | | | (Mettler |
| | | | | | | Toledo |
| | | | | | | MP50) |
| 81 | 2.739 | 99 | 546.6 | 547.1 | B | 218.2 |
| | | | | | | (Mettler |
| | | | | | | Toledo |
| | | | | | | MP50) |
| 82 | 3.246 | 99 | 565.5 | 566.1 | B | 211.6 |
| | | | | | | (Mettler |
| | | | | | | Toledo |
| | | | | | | MP50) |
| 83 | 3.01 | 99 | 576.594 | 577.2 | B | 149.7 |
| | | | | | | (Mettler |
| | | | | | | Toledo |
| | | | | | | MP50) |
| 84 | 3.214 | 99 | 561.579 | 562.2 | B | 208.2 |
| | | | | | | (Mettler |
| | | | | | | Toledo |
| | | | | | | MP50) |
| 85 | 3.182 | 99 | 561.579 | 562.2 | B | 198.3 |
| | | | | | | (Mettler |
| | | | | | | Toledo |
| | | | | | | MP50) |
| 86 | 2.247 | 99 | 561.579 | 562.1 | B | 211.6 |
| | | | | | | (Mettler |
| | | | | | | Toledo |
| | | | | | | MP50) |
| 87 | 3.01 | 99 | 579.57 | 580.1 | B | 188.63 |
| | | | | | | (Mettler |
| | | | | | | Toledo DSC3 |
| | | | | | | SFR System) |
| 88 | 3.114 | 99 | 577.579 | 578.1 | B | 193.2 |
| | | | | | | (Mettler |
| | | | | | | Toledo |
| | | | | | | MP50) |
| 89 | 2.511 | 99 | 547.553 | 547.8 | B | 236.6 |
| | | | | | | (Mettler |
| | | | | | | Toledo |
| | | | | | | MP50) |
| 90 | 2.892 | 98 | 567.971 | 568.1 | B | 226.6 |
| | | | | | | (Mettler |
| | | | | | | Toledo |
| | | | | | | MP50) |
| 91 | 3.532 | 99 | 567.971 | 568.1 | B | 213.3 |
| | | | | | | (Mettler |
| | | | | | | Toledo |
| | | | | | | MP50) |
| 92 | 2.988 | 99 | 547.553 | 548.2 | B | 117.9 |
| | | | | | | (Mettler |
| | | | | | | Toledo |
| | | | | | | MP50) |
| 93 | 3.30 | 96 | 559.563 | 560.2 | B | 241.7 |
| | | | | | | (Mettler |
| | | | | | | Toledo |
| | | | | | | MP50) |
| 94 | 2.67 | 99 | 559.563 | 560.2 | B | 234.9 |
| | | | | | | (Mettler |
| | | | | | | Toledo |
| | | | | | | MP50) |
| 95 | 3.114 | 98 | 561.579 | 562.2 | B | 102.8 |
| | | | | | | (Mettler |
| | | | | | | Toledo |
| | | | | | | MP50) |
| 96 | 2.331 | 96 | 469.538 | 470.2 | B | 183.2 |
| | | | | | | (Mettler |
| | | | | | | Toledo |
| | | | | | | MP50) |
| 97 | 3.062 | 99 | 578.542 | 579.1 | B | 238.3 |
| | | | | | | (Mettler |
| | | | | | | Toledo |
| | | | | | | MP50) |
| 98 | 3.215 | 96 | 574.578 | 575.2 | B | 199.9 |
| | | | | | | (Mettler |
| | | | | | | Toledo |
| | | | | | | MP50) |
| 99 | 2.531 | 99 | 505.592 | 506.2 | B | 266.7 |
| | | | | | | (Mettler |
| | | | | | | Toledo |
| | | | | | | MP50) |
| 100 | 1.73 | 97 | 441.528 | 442.2 | B | 189.9 |
| | | | | | | (Mettler |
| | | | | | | Toledo |
| | | | | | | MP50) |
| 101 | 1.870 | 99 | 485.581 | 486.2 | B | 186.5 |
| | | | | | | (Mettler |
| | | | | | | Toledo |
| | | | | | | MP50) |
| 102 | 3.40 | 99 | 573.59 | 574.2 | B | 193.2 |
| | | | | | | (Mettler |
| | | | | | | Toledo |
| | | | | | | MP50) |
| 103 | 3.402 | 99 | 580.97 | 581.0 | B | 240 |
| | | | | | | (Mettler |
| | | | | | | Toledo |
| | | | | | | MP50) |
| 104 | 3.607 | 96 | 579.982 | 580.1 | B | 229.9 |
| | | | | | | (Mettler |
| | | | | | | Toledo |
| | | | | | | MP50) |
| 105 | 1.833 | 96 | 469.581 | 470.2 | B | 183.2 |
| | | | | | | (Mettler |
| | | | | | | Toledo |
| | | | | | | MP50) |
| 106 | 2.824 | 98 | 533.645 | 534.2 | B | 195.2 |
| | | | | | | (Mettler |
| | | | | | | Toledo |
| | | | | | | MP50) |
| 107 | 3.110 | 99 | 509.526 | 510.2 | B | 250.1 |
| | | | | | | (Mettler |
| | | | | | | Toledo |
| | | | | | | MP50) |
| 108 | 3.112 | 98 | 547.672 | 548.2 | B | 231.6 |
| | | | | | | (Mettler |
| | | | | | | Toledo |
| | | | | | | MP50) |
| 109 | 2.669 | 99 | 549.645 | 550.2 | B | 258.5 |
| | | | | | | (Mettler |
| | | | | | | Toledo |
| | | | | | | MP50) |
| 110 | 2.806 | 99 | 548.541 | 549.1 | B | 251.7 |
| | | | | | | (Mettler |
| | | | | | | Toledo |
| | | | | | | MP50) |
| 111 | 2.788 | 99 | 510.514 | 511.2 | B | 253.7 |
| | | | | | | (Mettler |
| | | | | | | Toledo |
| | | | | | | MP50) |
| 112 | 3.06 | 96 | 577.554 | 578.1 | B | 251.7 |
| | | | | | | (Mettler |
| | | | | | | Toledo |
| | | | | | | MP50) |
| 113 | 3.19 | 99 | 588.486 | 589.1 | B | 218.3 |
| | | | | | | (Mettler |
| | | | | | | Toledo |
| | | | | | | MP50) |
| 114 | 3.41 | 99 | 587.497 | 588.1 | B | 189.8 |
| | | | | | | (Mettler |
| | | | | | | Toledo |
| | | | | | | MP50) |
| 115 | 2.964 | 99 | 572.483 | 573.1 | B | 191.5 |
| | | | | | | (Mettler |
| | | | | | | Toledo |
| | | | | | | MP50) |
| 116 | 2.833 | 99 | 554.492 | 555.1 | B | 181.3 |
| | | | | | | (Mettler |
| | | | | | | Toledo |
| | | | | | | MP50) |
| 117 | 2.862 | 99 | 566.983 | 567.2 | B | 196.5 |
| | | | | | | (Mettler |
| | | | | | | Toledo |
| | | | | | | MP50) |
| 118 | 3.488 | 99 | 566.983 | 567.1 | B | 220 |
| | | | | | | (Mettler |
| | | | | | | Toledo |
| | | | | | | MP50) |
| 119 | 3.578 | 99 | 572.483 | 573.1 | B | 203.2 |
| | | | | | | (Mettler |
| | | | | | | Toledo |
| | | | | | | MP50) |
| 120 | 3.471 | 99 | 554.492 | 555.1 | B | 184.9 |
| | | | | | | (Mettler |
| | | | | | | Toledo |
| | | | | | | MP50) |
| 121 | 3.658 | 96 | 586.509 | 587.1 | B | >300 |
| | | | | | | (Mettler |
| | | | | | | Toledo |
| | | | | | | MP50) |
| 122 | 3.300 | 99 | 573.471 | 574.1 | B | 223.2 |
| | | | | | | (Mettler |
| | | | | | | Toledo |
| | | | | | | MP50) |
| 123 | 2.58 | 99 | 563.527 | 564.1 | B | 223.2 |
| | | | | | | (Mettler |
| | | | | | | Toledo |
| | | | | | | MP50) |
| 124 | 2.818 | 99 | 566.531 | 567.2 | B | 176.4 |
| | | | | | | (Mettler |
| | | | | | | Toledo |
| | | | | | | MP50) |
| 125 | 3.015 | 98 | 578.542 | 479.1 | B | 194.9 |
| | | | | | | (Mettler |
| | | | | | | Toledo |
| | | | | | | MP50) |
| 126 | 2.755 | 95 | 548.541 | 549.2 | B | 250.1 |
| | | | | | | (Mettler |
| | | | | | | Toledo |
| | | | | | | MP50) |
| 127 | 2.590 | 99 | 548.541 | 549.2 | B | 208.2 |
| | | | | | | (Mettler |
| | | | | | | Toledo |
| | | | | | | MP50) |
| 128 | 2.772 | 97 | 560.551 | 561.2 | B | 199.9 |
| | | | | | | (Mettler |
| | | | | | | Toledo |
| | | | | | | MP50) |
| 129 | 2.459 | 99 | 562.567 | 563.1 | B | 173.1 |
| | | | | | | (Mettler |
| | | | | | | Toledo |
| | | | | | | MP50) |
| 130 | 2.641 | 95 | 579.53 | 580.1 | B | 228.3 |
| | | | | | | (Mettler |
| | | | | | | Toledo |
| | | | | | | MP50) |
| 131 | 2.540 | 99 | 549.529 | 550.1 | B | 273.4 |
| | | | | | | (Mettler |
| | | | | | | Toledo |
| | | | | | | MP50) |

1. Biological Assays/Pharmacological Examples

MIC Determination for Testing Compounds Against M. tuberculosis.

### Test 1

Test compounds and reference compounds were dissolved in DMSO and 1 μl of solution was spotted per well in 96 well plates at 200× the final concentration. Column 1 and column 12 were left compound-free, and from column 2 to 11 compound concentration was diluted 3-fold. Frozen stocks of Mycobacterium tuberculosis strain (EH4.0 in this case; other strains may be used e.g. H37Rv) expressing green-fluorescent protein (GFP) were previously prepared and titrated. To prepare the inoculum, 1 vial of frozen bacterial stock was thawed to room temperature and diluted to 5×10 exp5 colony forming units per ml in 7H9 broth. 200 μl of inoculum, which corresponds to 1×10 exp5 colony forming units, were transferred per well to the whole plate, except column 12. 200 μl 7H9 broth were transferred to wells of column 12. Plates were incubated at 37° C. in plastic bags to prevent evaporation. After 7 days, fluorescence was measured on a Gemini EM Microplate Reader with 485 excitation and 538 nm emission wavelengths and IC₅₀ (or AC₅₀) and/or pIC₅₀ values (or the like, e.g. IC₅₀, IC₉₀, pIC₉₀, etc) were (or may be) calculated.

### Test 2

Appropriate solutions of experimental/test and reference compounds were made in 96 well plates with 7H9 medium. Samples of Mycobacterium tuberculosis strain H37Rv were taken from cultures in logarithmic growth phase. These were first diluted to obtain an optical density of 0.3 at 600 nm wavelength and then diluted 1/100, resulting in an inoculum of approximately 5×10 exp5 colony forming units per ml. 100 μl of inoculum, which corresponds to 5×10 exp4 colony forming units, were transferred per well to the whole plate, except column 12. Plates were incubated at 37° C. in plastic bags to prevent evaporation. After 7 days, resazurin was added to all wells. Two days later, fluorescence was measured on a Gemini EM Microplate Reader with 543 excitation and 590 nm emission wavelengths and MIC₅₀ and/or pIC₅₀ values (or the like, e.g. IC₅₀, IC₉₀, pIC₉₀, etc) were (or may be) calculated.

### Test 3: Time Kill Assays

Bactericidal or bacteriostatic activity of the compounds can be determined in a time kill kinetic assay using the broth dilution method. In this assay, the starting inoculum of M. tuberculosis (strain H37Rv and H37Ra) is 10⁶ CFU/ml in Middlebrook (1×) 7H9 broth. The test compounds are tested alone or in combination with another compound (e.g. a compound with a different mode of action, such as with a cytochrome bd inhibitor) at a concentration ranging from 10-30 μM to 0.9-0.3 μM respectively. Tubes receiving no antibacterial agent constitute the culture growth control. The tubes containing the microorganism and the test compounds are incubated at 37° C. After 0, 1, 4, 7, 14 and 21 days of incubation samples are removed for determination of viable counts by serial dilution (100 to 10⁶) in Middlebrook 7H9 medium and plating (100 l) on Middlebrook 7H11 agar. The plates are incubated at 37° C. for 21 days and the number of colonies are determined. Killing curves can be constructed by plotting the log₁₀ CFU per ml versus time. A bactericidal effect of a test compound (either alone or in combination) is commonly defined as 2-log₁₀ decrease (decrease in CFU per ml) compared to Day 0. The potential carryover effect of the drugs is limited by using 0.4% charcoal in the agar plates, and by serial dilutions and counting the colonies at highest dilution possible used for plating.

### Results

Compounds of the invention/examples, for example when tested in Test 1 (and/or Test 2) described above, may typically have a pIC₅₀ from 3 to 10 (e.g. from 4.0 to 9.0, such as from 5.0 to 8.0)

2. Biological Results
Compounds of the examples were tested in Test 1 (and/or in Test 2) described above (in section “Pharmacological Examples”) and the following results were obtained:

Biological data table

| pIC50 | cpd nbr | AC₅₀ | cpd nbr |
|---|---|---|---|
| 8.11 | 1 | | 83 |
| 8.00 | 2 | 0.0076 | 84 |
| 7.21 | 3 | 0.018 | 85 |
| 7.90 | 4 | 0.030 | 86 |
| 6.64 | 5 | 0.036 | 87 |
| 7.08 | 6 | 0.00866 | 88 |
| 8.07 | 7 | | 89 |
| 7.58 | 8 | | 90 |
| 6.44 | 9 | | 91 |
| <6.301 | 10 | >0.50 | 92 |
| 6.35 | 11 | | 93 |
| 7.03 | 12 | 0.063 | 94 |
| <6.301 | 13 | | 95 |
| 6.98 | 14 | | 96 |
| 6.30 | 15 | | 97 |
| 6.30 | 16 | 0.017 | 98 |
| 6.71 | 17 | | 99 |
| 6.82 | 18 | | 100 |
| 6.48 | 19 | | 101 |
| 6.30 | 20 | | 102 |
| 7.39 | 21 | | 103 |
| 7.35 | 22 | 0.11 | 104 |
| 7.49 | 23 | | 105 |
| 8.05 | 24 | | 106 |
| 6.74 | 25 | | 107 |
| 6.61 | 26 | | 108 |
| 6.86 | 27 | | 109 |
| 7.27 | 28 | | 110 |
| 7.66 | 29 | | 111 |
| 7.38 | 30 | 0.0095 | 112 |
| 7.62 | 31 | 0.032 | 113 |
| 7.22 | 32 | 0.0098 | 114 |
| 7.89 | 33 | | 115 |
| 7.90 | 34 | | 116 |
| <6.301 | 35 | | 117 |
| 7.79 | 36 | | 118 |
| 6.34 | 37 | | 119 |
| 6.55 | 38 | | 120 |
| 6.91 | 39 | | 121 |
| 6.30 | 40 | | 122 |
| 7.34 | 41 | 0.018 | 123 |
| 8.30 | 42 | | 124 |
| 6.55 | 43 | | 125 |
| 8.66 | 44 | | 126 |
| 7.82 | 45 | 0.049 | 127 |
| 6.55 | 46 | | 128 |
| 7.70 | 47 | | 129 |
| 7.59 | 48 | | 130 |
| 6.30 | 49 | | 131 |
| 6.77 | 50 | | |
| 7.38 | 51 | | |
| 6.68 | 52 | | |
| 6.92 | 53 | | |
| 6.88 | 54 | | |
| 7.30 | 55 | | |
| 7.13 | 56 | | |
| 7.58 | 57 | | |
| 6.91 | 58 | | |
| 6.96 | 59 | | |
| 7.03 | 60 | | |
| 6.41 | 61 | | |
| 6.76 | 62 | | |
| 6.72 | 63 | | |
| 6.88 | 64 | | |
| 6.48 | 65 | | |
| 6.57 | 66 | | |
| 6.79 | 67 | | |
| <6.3* | 68 | | |
| 7.82 | 69 | | |
| 7.04 | 70 | | |
| <6.3* | 71 | | |
| <6.3 | 72 | | |
| <6.3 | 73 | | |
| 6.51 | 74 | | |
| <6.3 | 75 | | |
| <6.3 | 76 | | |
| 6.34 | 77 | | |
| <6.3 | 78 | | |
| 8.17 | 79 | | |
| 8.06 | 80 | | |
| 7.69 | 81 | | |
| 8.55 | 82 | | |

3. Further Data on Representative Compounds of the Invention/Examples

The compounds of the invention/examples may have advantages associated with in vitro potency, kill kinetics (i.e. bactericidal effect) in vitro, PK properties, food effect, safety/toxicity (including liver toxicity, coagulation, 5-LO oxygenase), metabolic stability, Ames II negativity, MNT negativity, aqueous based solubility (and ability to formulate) and/or cardiovascular effect e.g. on animals (e.g. anesthetized guinea pig). Data that is generated/calculated may be obtained using standard methods/assays, for instance that are available in the literature or which may be performed by a supplier (e.g. Microsomal Stability Assay—Cyprotex, Mitochondrial toxicity (Glu/Gal) assay—Cyprotex, as well as literature CYP cocktail inhibition assays). GSH can be measured (reactive metabolites, glucuronidation) to observe if a dihydrodiol is observed by LCMS (fragmentation ions), which would correspond to a dihydroxylation on the core heterocycle.

This following data were generated:

### Compound 2

LM Clint μL/min/mg h/m/r/d=9.3<7.7/<7.7/<7.7
MDCK AB+inh: 32.5
MDCK BA/AB: 12.6
PPB h/m % free: 1.17/0.54
Eq sol pH 2/7 (μM): 0.99 am/<0.13 am
Fassif/Fessif (μM): <5/24.4
CYPS IC₅₀ μM: all>20
sync hERG/Na/Ca (IC₅₀ μM)>30/>10/>10
CTCM: clean up to 10 μM
HCS: 32.7 μM NC
AMES II: 1
Glu/Gal: >100/>100

### Compound 7

LM Clint μL/min/mg h/m=22.6/<7,7
MDCK AB+inh: 21.4
MDCK BA/AB: 61.7
Eq sol pH 2/7 (μM): 125 c/1.62 c
sync hERG/Na/Ca (IC₅₀ μM)>30/>10/>10
CTCM: clean up to 10 μM
CYPS IC₅₀ μM: 2C9 15.5; others>20
HCS: >21 μM
Glu/Gal: >200/>200

### Compound 79

LM CLint uL/min/mg h/m=39.8/13.2
Hep t1/2 min h/m=−/43.3
MDCK AB+inh=42.7
MDCK BA/AB=--
Sol pH 2/4 uM: 574 am/0.062 am
Fassif/Fessif uM: 5.6/29.3
CYPS IC₅₀ uM: 2C19 14.4; 2C9 17.7, others>20
sync hERG/Na/Ca (IC₅₀ uM) 30.2/>10/>10
AMES II: 1
Glu/Gal: >200/>200

### Compound 82

LM Clint uL/min/mg h/m=231/28
Hep t1/2 min h/m=-/16.5
MDCK AB+inh=16.2
MDCK AB/BA=--
Sol pH 2/4 uM: 12.3 c/<0.02 c
Fassif/Fessif uM: 24.5/8.8
CYPS IC₅₀ uM: 2C8 10.6, others>19.5
sync hERG/Na/Ca (IC₅₀ uM) 20.4/>10/>10
AMES: 1
Glu/Gal: >25/>25
   The Following Further Data/Results were Generated

Compound 2 and Compound 7
were found to have low mitotoxicity (<2 in the Glu/Gal assay)—hence no mitotoxicity alerts

### Further Mitotoxicity Data

| cpd number | IC50, gal | IC50, glu | Δ IC50, glu/IC50, gal | Score |
|---|---|---|---|---|
| 1 | >199.986 | >199.986 | | |
| 2 | >100 | >100 | | |
| 3 | 73.06341 | 66.23694 | 0.91 | [x]1 |
| 4 | >100 | >100 | | |
| 5 | 116.6541 | 111.6092 | 0.96 | [x]1 |
| 6 | 134.4003 | 88.45046 | 0.66 | [x]1 |
| 7 | >199.986 | >199.986 | | |
| 8 | 41.71573 | 51.22711 | 1.23 | [x]1 |
| 9 | 19.90216 | 22.89812 | 1.15 | [x]1 |
| 10 | >199.986 | >199.986 | | |
| 11 | 68.21815 | >199.986 | >2.93 | [x]2 |
| 13 | 23.53965 | 29.51888 | 1.25 | [x]1 |
| 15 | >199.986 | >199.986 | | |
| 16 | >199.986 | >199.986 | | |
| 17 | >199.986 | >199.986 | | |
| 18 | 183.6116 | >199.986 | >1.09 | [x]1 |
| 19 | >24.9977 | >24.9977 | | |
| 20 | 153.6031 | >199.986 | >1.3 | [x]1 |
| 21 | 59.41554 | 97.85885 | 1.65 | [x]1 |
| 22 | 62.60368 | 94.73273 | 1.51 | [x]1 |
| 23 | >199.986 | >199.986 | | |
| 24 | >199.986 | >199.986 | | |
| 25 | 22.11058 | >50.0034 | >2.26 | [x]2 |
| 26 | >199.986 | >199.986 | | |
| 27 | 44.5451 | 104.3998 | 2.34 | [x]2 |
| 28 | >199.986 | >199.986 | | |
| 29 | >199.986 | >199.986 | | |
| 30 | 71.07221 | 95.7194 | 1.35 | [x]1 |
| 31 | >199.986 | >199.986 | | |
| 32 | 15.72896 | >199.986 | >12.72 | [x]3 |
| 33 | >199.986 | 195.569 | <0.98 | [x]1 |
| 34 | 44.19774 | >50.0034 | >1.13 | [x]0 |
| 35 | 40.06821 | >50.0034 | >1.25 | [x]0 |
| 36 | 79.46939 | >100 | >1.26 | [x]0 |
| 37 | >199.986 | >199.986 | | |
| 38 | 8.679605 | >100 | >11.52 | [x]3 |
| 39 | 30.98131 | >199.986 | >6.46 | [x]3 |
| 40 | >199.986 | >199.986 | | |
| 41 | >199.986 | >199.986 | | |
| 42 | >24.9977 | >24.9977 | | |
| 43 | >199.986 | >199.986 | | |
| 44 | >199.986 | >199.986 | | |
| 45 | >100 | >100 | | |
| 46 | >199.986 | >199.986 | | |
| 47 | >199.986 | >199.986 | | |
| 48 | >199.986 | >199.986 | | |
| 49 | 70.82722 | >199.986 | >2.82 | [x]2 |
| 50 | >199.986 | >199.986 | | |
| 51 | >199.986 | >199.986 | | |
| 52 | >199.986 | >199.986 | | |
| 53 | >199.986 | >199.986 | | |
| 54 | >199.986 | >199.986 | | |
| 55 | 173.7001 | >199.986 | >1.15 | [x]1 |
| 56 | >199.986 | >199.986 | | |
| 57 | >199.986 | >199.986 | | |
| 58 | 58.18353 | >199.986 | >3.44 | [x]2 |
| 59 | 17.5752 | >199.986 | >11.38 | [x]3 |
| 60 | 56.22117 | >199.986 | >3.56 | [x]2 |
| 61 | >199.986 | >199.986 | | |
| 62 | >199.986 | >199.986 | | |
| 63 | | | | |
| 64 | >199.986 | >199.986 | | |
| 65 | >199.986 | >199.986 | | |
| 66 | >199.986 | >199.986 | | |
| 67 | 148.7647 | >199.986 | >1.34 | [x]1 |
| 68 | >100 | >100 | | |
| 69 | 15.34617 | 174.4616 | 11.37 | [x]3 |
| 70 | >50.0034 | >50.0034 | | |
| 71 | >199.986 | >199.986 | | |
| 72 | >199.986 | >199.986 | | |
| 73 | >199.986 | >199.986 | | |
| 74 | | | | |
| 75 | >199.986 | >199.986 | | |
| 76 | >199.986 | >199.986 | | |
| 77 | >199.986 | >199.986 | | |
| 78 | >199.986 | >199.986 | | |
| 79 | >50.0034 | >50.0034 | | |
| 80 | >199.986 | >199.986 | | |
| 81 | >199.986 | >199.986 | | |
| 82 | >24.9977 | >24.9977 | | |
| 83 | 126.5027 | 142.1674 | 1.12 | [x]1 |
| 84 | 62.54604 | >199.986 | >3.2 | [x]2 |
| 85 | 30.06767 | >199.986 | >6.65 | [x]3 |
| 86 | >100 | >100 | | |
| 87 | 25.42143 | 85.82226 | 3.38 | [x]2 |
| 88 | | | | |
| 89 | >199.986 | >199.986 | | |
| 90 | >199.986 | >199.986 | | |
| 91 | 15.5704 | >199.986 | >12.85 | [x]3 |
| 92 | 132.8924 | 194.8498 | 1.47 | [x]1 |
| 93 | >199.986 | >199.986 | | |
| 94 | >199.986 | >199.986 | | |
| 95 | 117.4897 | >199.986 | >1.7 | [x]1 |
| 96 | >199.986 | >199.986 | | |
| 97 | >24.9977 | >24.9977 | | |
| 98 | 97.81374 | 177.4598 | 1.81 | [x]1 |
| 99 | >199.986 | >199.986 | | |
| 100 | >199.986 | >199.986 | | |
| 101 | >199.986 | >199.986 | | |
| 102 | 29.90198 | >199.986 | >6.69 | [x]3 |
| 103 | 38.60111 | >50.0034 | >1.3 | [x]0 |
| 104 | >50.0034 | >50.0034 | | |
| 106 | >100 | >100 | | |
| 107 | 44.22827 | >50.0034 | >1.13 | [x]0 |
| 108 | 38.61002 | >50.0034 | >1.29 | [x]0 |
| 109 | >50.0034 | >50.0034 | | |
| 110 | >100 | >100 | | |
| 112 | >100 | >100 | | |
| 113 | >100 | >100 | | |
| 114 | >12.4997 | >12.4997 | | |
| 115 | 64.84853 | >199.986 | >3.08 | [x]2 |
| 116 | 20.08169 | >199.986 | >9.96 | [x]3 |
| 117 | >50.0034 | >50.0034 | | |
| 118 | 127.0867 | >199.986 | >1.57 | [x]1 |
| 119 | 130.0769 | >199.986 | >1.54 | [x]1 |
| 120 | 24.95744 | >199.986 | >8.01 | [x]3 |
| 121 | 46.35537 | >199.986 | >4.31 | [x]2 |
| 122 | >100 | >100 | | |
| 123 | >100 | >100 | | |
| 124 | >199.986 | >199.986 | | |
| 125 | >199.986 | >199.986 | | |
| 126 | >199.986 | >199.986 | | |
| 127 | >199.986 | >199.986 | | |
| 128 | >199.986 | >199.986 | | |
| 129 | >199.986 | >199.986 | | |
| 130 | >199.986 | >199.986 | | |
| 131 | >100 | >100 | | |

In the table above, “[x]1” is “negative”, which means that in the test, it was found to have low mitotoxicity (and hence no mitotoxicity alerts), “[x]3” is “positive”, which means that there were some mitotoxicity alerts and “[x]0” is “inconclusive”, which means that no accurate conclusion could be drawn, e.g. due to issues with the compound being tested in the assay, e.g. solubility or precipitation issues (e.g. compound may not be soluble enough or may precipitate).

In view of the data above, compounds of the invention/examples may be found to be advantageous as no mitotoxicity alerts were observed (e.g. in the Glu/Gal assay).

### Further Data

### PPB % Free (Human at 1 μm)

Compound 1: 0.047
Compound 2: 1.17
Compound 42: 2.90

### CLint Microsomes (μL/Min/Mg) in Dog (d), Human (h), Mouse (m) and Rat (r) (all at 1 μm)

Compound 1: 30.3 (h), 49.2 (m)
Compound 2: <7.7 (d), 9.3 (h), <7.7 (m), <7.7 (r)
Compound 7: 22.6 (h), <7.7 (m)
Compound 15: 16.2 (h), 50.6 (m)
Compound 18: 20.1 (h), 36.9 (m)
Compound 24:169 (h), 76.3 (m)
Compound 42: 47.1 (h), 20.4 (m)
Compound 44: 176 (h), 20 (m)
Compound 47: 58.5 (h), 13.9 (m)
Compound 66: 39.6 (h), 92.3 (m)
Compound 79: 298 (h), 74.3 (m)
Compound 80: 39.8 (h), 13.2 (m)
Compound 81: 189 (d), >347 (h), 74.8 (m), 80.7 (r)
Compound 82: 231 (h), 28 (m)
Compound 84: 205 (h), 21.2 (m)
Compound 94: 98.1 (h), 18.1 (m)
Compound 98: 51.8 (h), 26.9 (m)
Compound 106: 111 (h), 83.4 (m)
Compound 127: 31.5 (h), 9.29 (m)
Compound 129: 50.4 (h), 29.4 (m)

Compounds disclosed herein may have the advantage that:
No in vitro cardiotoxicity is observed (for example either due to the CVS results or due to the Glu/Gal assay results);
No reactive metabolite formation is observed (e.g. GSH), for instance as no unwanted reactive metabolites are formed and/or the formation of reactive metabolites was blocked; and/or
There is a relatively higher unbound fraction, for instance as compared to other compounds, for instance prior art compounds.

Certain compounds may also have the additional advantage that they do not form degradants (e.g. that are undesired or may elicit unwanted side-effects).

Compounds, may have the advantage that a faster oral absorption and improved bioavailability are displayed.

### Chemical Stability Testing

Compounds disclosed herein may have the advantage that they are chemically more stable than other compounds (e.g. than other known compounds), for instance as tested in the chemical stability assay described below.

### Preliminary Protocol

Add 3 μl of a 10 mM DMSO stock solution to 1 ml of the following solvents in a 1.5 ml HPLC vial.
DMSO (reference solution)
H₂O/Acetonitril 1/1 (assay solution)
0.1N HCl/Acetonitril 1/1 (assay solution)
Mix well, store them on the bench for 72h
Analyse the samples with LCMS
Compare the chromatograms of the two assay solutions with the reference solution and report the additional peaks as degradation peaks

For instance, the following chemical stability results (in % by LCMS) were observed:
Compound 2: conditions—0.065 mg/mL in SGF with 20% ACN−results−purity=99.56% (at 0 hr), 99.38% (at 0.25 hr), 99.21% (at 0.5 hr), 98.89% (at 1 hr), 98.28% (at 2 hr), 97.1% (at 4 hr) (t1/2=112.81)
Compound 6: conditions—0.052 mg/mL in SGF with 33.3% ACN−results−purity=99.88 (and remained so, up to 4 hrs)
Compound 2: DMSO (72 hr, rt)=100%; ACN/0.1N HCl (pH 1.6; 72 hr, rt)=90.52%
Compound 10: DMSO (72 hr, rt)=97.03%; ACN/0.1N HCl (pH 1.6; 72 hr, rt)=100%
Compound 7: DMSO (72 hr, rt)=100%; ACN/0.1N HCl (pH 1.6; 72 hr, rt)=100%
Compound 14: DMSO (72 hr, rt)=100%; ACN/0.1N HCl (pH 1.6; 72 hr, rt)=100%
Compound 15: DMSO (72 hr, rt)=97.03%; ACN/0.1N HCl (pH 1.6; 72 hr, rt)=97.49%
Compound 12: DMSO (72 hr, rt)=96.14%; ACN/0.1N HCl (pH 1.6; 72 hr, rt)=97.06%
Compound 6: ACN/H₂O (48 hr, rt)=100%; ACN/0.1N HCl (pH 1.6; 48 hr, rt)=100%
Compound 47: ACN/H₂O (48 hr, rt)=99%; ACN/0.1N HCl (pH 1.6; 48 hr, rt)=100%
Compound 42: ACN/H₂O (48 hr, rt)=100%; ACN/0.1N HCl (pH 1.6; 48 hr, rt)=100%
Compound 66: DMSO (0 hr, rt)=91%; ACN/H₂O (48 hr, rt)=98%; ACN/0.1N HCl (pH 1.6; 48 hr, rt)=98%
Compound 24: DMSO (0 hr and 48 hr, rt)=100%; ACN/0.1N HCl (pH 1.6; 0 hr and 48 hr, rt)=100%; ACN/0.1N NaOH (pH 9-10; 0 hr and 48 hr, rt)=89.46% and 43.8%
Compound 80: DMSO (0 hr and 48 hr, rt)=100%; ACN/0.1N HCl (pH 1.6; 0 hr and 48 hr, rt)=100%; ACN/0.1N NaOH (pH 9-10; 0 hr and 48 hr, rt)=76.8% and 16.8%
Compound 79: DMSO (0 hr, rt)=100%; ACN/H₂O (48 hr, rt)=100%; ACN/0.1N HCl (pH 1.6; 48 hr, rt)=100%
Compound 44: ACN/H₂O (48 hr, rt)=100%; ACN/0.1N HCl (pH 1.6; 48 hr, rt)=100%
Compound 82: ACN/H₂O (48 hr, rt)=100%; ACN/0.1N HCl (pH 1.6; 48 hr, rt)=100%
Compound 81: DMSO (0 hr, rt)=95%; ACN/H₂O (48 hr, rt)=100%; ACN/0.1N HCl (pH 1.6; 48 hr, rt)=100%

This showed that, under the tested conditions, the compounds were stable, and mostly not susceptible to unwanted degradation in acidic media (or alkaline media, as the case may be).

1. A compound of formula (I); 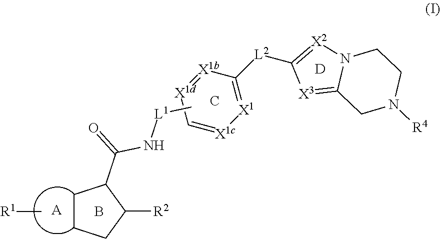
wherein:
A is a 5- or 6-membered ring, which is aromatic or non-aromatic, and optionally containing 1 or 2 heteroatoms that are nitrogen, sulfur or oxygen;
B is a 5-membered aromatic ring containing 1 or 2 nitrogen heteroatoms;
X¹ is ═N— or ═C(R^{10a})—;
X^{1b} is ═N— or ═C(R³)—.
X^{1c} is ═C(R^{10a}) or ═N—;
X^{1d} is ═C(R^{10a}) or ═N—, and wherein a maximum of two of X¹, X^{1b}, X^{1c} and C^{1d} are ═N—;
one of X² and X³ in the D ring is ═N— and the other is ═N— or ═C(R^{10b})—;
L¹ is a linker group;
L² is an optional linker group;
R¹ is one or more optional substituents independently that are halo, —R^{5a}, —O—R^{5b}, —C(═O)—R^{5c}, —C(═O)—N(R⁶)(R⁷), —CN or —N(R^{6a})R^{6b}; or any two R¹ groups are taken together when attached to adjacent atoms of the A ring to form a 5- or 6-membered ring optionally containing one or two heteroatoms, and which ring is optionally substituted by one or two C₁₋₃ alkyl substituents;
R² is —C₁₋₄ alkyl optionally substituted by one or more substituents that are halo or —OC₁₋₃ alkyl;
R³ is a substituent that is H, F, —C₁₋₃ alkyl or —O—C₁₋₃ alkyl;
R⁴ is H, —R^{8a}, —C(═O)—R^{8b}, —SO₂—R⁹ or Het¹;
R^{5a} and R^{5b} independently are hydrogen or —C₁₋₄ alkyl optionally substituted by one or more substituents that are halo, —O—CH₃ or phenyl;
R⁵ is —C₁₋₃ alkyl;
R⁶ and R⁷ are independently H or —C₁₋₃ alkyl;
R^{6a} and R^{6b} independently are H, C₁₋₆ alkyl or R^{6a} and R^{6b} are linked together to form a 3- to 6-membered ring;
R^{8a} is —C₁₋₄ alkyl, optionally substituted by one or more substituents that are halo, —OC₁₋₃ alkyl, —CN or Het²;
R^{8b} is hydrogen or —C₁₋₃ alkyl optionally substituted by one or more fluoro atoms;
R⁹ is Het³, —N(R^{6c})R^{6d} or —C₁₋₄ alkyl optionally substituted by one or more substituents that are halo or —O—CH₃;
R^{6c} and R^{6d} independently represent are H, C₁₋₆ alkyl or R^{6c} and R^{6d} are linked together to form a 3- to 6-membered ring;
R^{10a} and R^{10b} independently represent are H, halo, C₁₋₄ alkyl (optionally substituted by one or more substituent(s) that are fluoro, —CN, —R^{11a}, —OR^{11b}, —N(R^{11c})R^{11d}, and/or —C(O)N(R^{11e})R^{11f} or —O—C₁₋₄ alkyl itself optionally substituted by one or more substituent(s) that are fluoro, —R^{11g}, —OR^{11h} or —N(R¹¹ⁱ)R^{11j};
R^{11a}, R^{11b}, R^{11c}, R^{11d}, R^{11e}, R^{11f}, R^{11g}, R^{11h}, R¹¹ⁱ and R^{11j} independently are hydrogen or C₁₋₃ alkyl optionally substituted by one or more fluoro atoms;
R^{12a} and R^{12b} independently represent are hydrogen or C₁₋₃ alkyl; or R^{12a} and R^{12b} are linked together to form a 3- to 6-membered ring;
R^{12c} and R^{12d} independently are hydrogen or C₁₋₃ alkyl; or R^{12c} and R^{12d} are linked together to form a 3- to 6-membered ring;
Het¹, Het² and Het³ independently are a 5- or 6-membered aromatic ring containing one or two heteroatoms, optionally substituted by one or more substituents that are halo or C₁₋₃ alkyl optionally substituted by one or more fluoro atoms,
or a pharmaceutically-acceptable salt thereof.

2. A compound of formula (I): 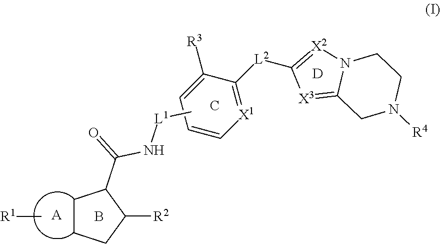
wherein:
A is a 5- or 6-membered ring, which is aromatic or non-aromatic, and optionally containing 1 or 2 heteroatoms that are nitrogen, sulfur or oxygen;
B is a 5-membered aromatic ring containing 1 or 2 nitrogen heteroatoms;
X¹ is ═N— or ═C(R^{10a})—;
one of X² and X³ is ═N— and the other is ═N— or ═C(R^{10b})—;
L¹ is a linker group;
L² is an optional linker group;
R¹ is one or more optional substituents independently that are halo, —R^{5a}, —O—R^{5b}, —C(═O)—R^{5c}, —C(═O)—N(R⁶)(R⁷), —CN or —N(R^{6a})R^{6b}; or any two R¹ groups are taken together when attached to adjacent atoms of the A ring to form a 5- or 6-membered ring optionally containing one or two heteroatoms, and which ring is optionally substituted by one or two C₁₋₃ alkyl substituents;
R² is —C₁₋₄ alkyl optionally substituted by one or more substituents that are halo or —OC₁₋₃ alkyl;
R³ is a substituent that is H, F, —C₁₋₃ alkyl —O—C₁₋₃ alkyl;
R⁴ is H, —R^{8a}, —C(═O)—R^{8b}, —SO₂—R⁹ or Het¹;
R^{5a} and R^{5b} independently are hydrogen or —C₁₋₄ alkyl optionally substituted by one or more substituents that are halo, —O—CH₃ or phenyl;
R^{5c} is —C₁₋₃ alkyl;
R⁶ and R⁷ are independently H or —C₁₋₃ alkyl;
R^{6a} and R^{6b} independently are H, C₁₋₆ alkyl or R^{6a} and R^{6b} are linked together to form a 3- to 6-membered ring;
R^{8a} is —C₁₋₄ alkyl, optionally substituted by one or more substituents that are halo, —OC₁₋₃ alkyl, —CN or Het²;
R^{8b} is hydrogen or —C₁₋₃ alkyl optionally substituted by one or more fluoro atoms;
R⁹ is Het³, —N(R^{6c})R^{6d} or —C₁₋₄ alkyl optionally substituted by one or more substituents that are halo or —O—CH₃;
R^{6c} and R^{6d} independently are H, C₁₋₆ alkyl or R^{6c} and R^{6d} are linked together to form a 3- to 6-membered ring;
R^{10a} and R^{10b} independently are H, halo, C₁₋₄ alkyl itself optionally substituted by one or more substituent(s) that are fluoro, —CN, —R^{11a}, —OR^{11b}, —N(R^{11c})R^{11d} or —C(O)N(R^{11e})R^{11f} or —O—C₁₋₄ alkyl itself optionally substituted by one or more substituent(s) that are fluoro, —R^{11g}, —OR^{11h} or —N(R¹¹ⁱ)R^{11j};
R^{11a}, R^{11b}, R^{11c}, R^{11a}, R^{11e}, R^{11f}, R^{11g}, R^{11h}, R¹¹ⁱ and R^{11j} independently are hydrogen or C₁₋₃ alkyl optionally substituted by one or more fluoro atoms;
R^{12a} and R^{12b} independently are hydrogen or C₁₋₃ alkyl; or R^{12a} and R^{12b} are linked together to form a 3- to 6-membered ring;
R^{12c} and R^{12d} independently are hydrogen or C₁₋₃ alkyl; or R^{12c} and R^{12d} are linked together to form a 3- to 6-membered ring;
Het¹, Het² and Het³ independently are a 5- or 6-membered aromatic ring containing one or two heteroatoms, optionally substituted by one or more substituents that are halo or C₁₋₃ alkyl (optionally substituted by one or more fluoro atoms,
or a pharmaceutically-acceptable salt thereof,

3. The compound according to claim 1,
wherein ring A is as follows:

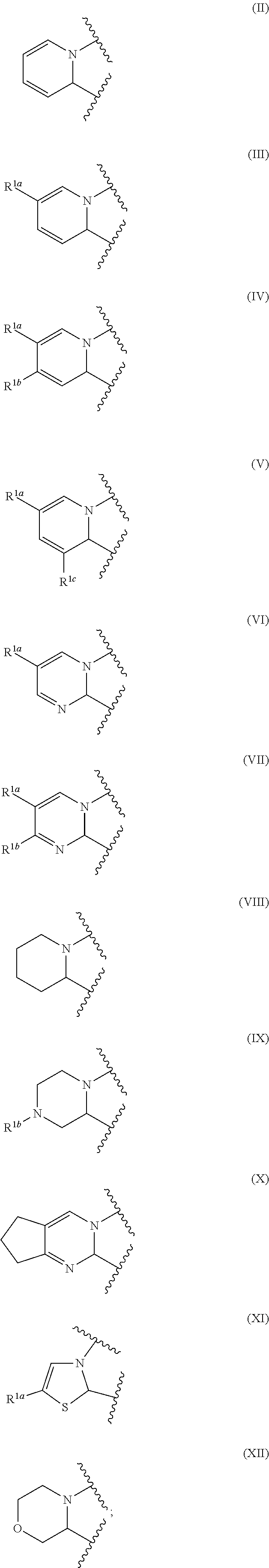

4. The compound according to claim 1, wherein ring A and ring B may be represented are as follows:

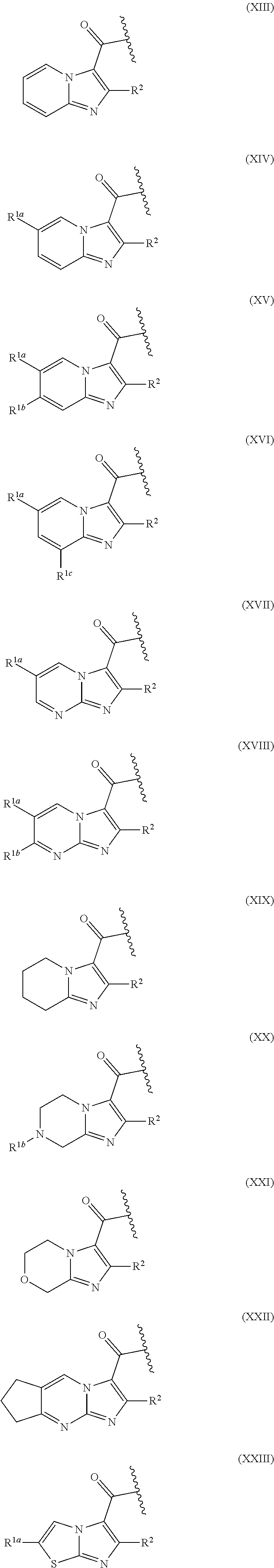

5. The compound according to claim 1, wherein ring C is as follows:

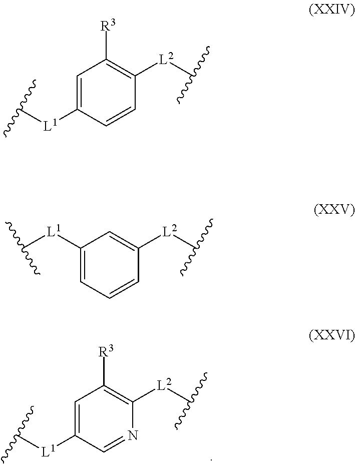

6. The compound according to claim 1, wherein ring D is as follows:

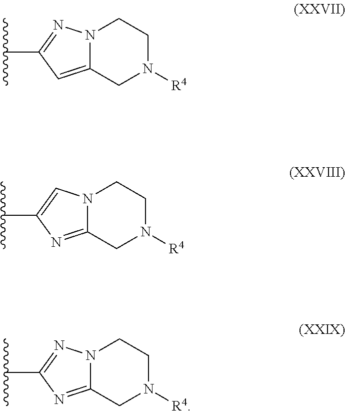

7. The compound according to claim 1, wherein L¹ is a linker group that is —CH₂—, —CH₂—CH₂—, or —C(R^{12a})(R^{12b})—, wherein R^{12a} and R^{12b} each independently are —CH₃ or are linked together to form a 3-membered ring.

8. The compound according to claim 1, wherein L² is a linker group that is a direct bond, —CH₂—, or a 4- or 5- or 6-membered non-aromatic ring optionally containing one or two nitrogen atom(s).

9. (canceled)

10. A pharmaceutical composition comprising a pharmaceutically acceptable carrier and, as active ingredient, a therapeutically effective amount of the compound of claim 1.

11. (canceled)

12. (canceled)

13. A method treating a mycobacterial infection in a patient, comprising administering a therapeutically effective amount of the compound of claim 1 to the patient.

14. A combination of (a) the compound of claim 1, and (b) one or more other anti-mycobacterial agent.

15. A product containing (a) the compound of claim 1, and (b) one or more other anti-mycobacterial agent, as a combined preparation for simultaneous, separate or sequential use in treating a bacterial infection.

16. A process for preparing the compound of formula (I) of claim 1, comprising (i) reacting a compound of formula (XXX): 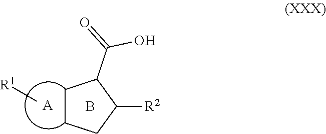
with a compound of formula (XXXI): 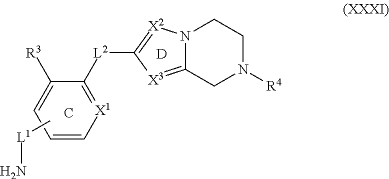
(ii) coupling of a compound of formula (XXXII) 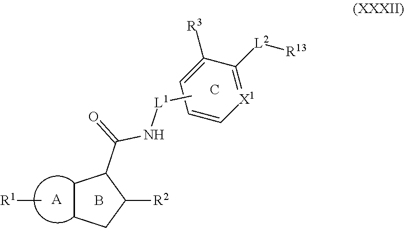
wherein R¹³ is suitable leaving group, with a compound of formula (XXXIII): 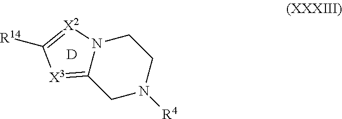
wherein R¹⁴ is a suitable leaving group.

17. The compound according to claim 1, wherein:
(i) the C ring is phenyl, pyridyl, pyrimidinyl;
(ii) L¹ is —C(R^{12a})(R^{12b})— or C₂₋₄ alkylene optionally substituted by one or more substituents that are halo or —OC₁₋₃ alkyl;
(iii) L¹ is situated para or meta relative to L²;
(iv) L¹ is attached to X^{1d} or the carbon atom in between X^{1d} and X^{1c};
(v) L² is a direct bond, —O—, —OCH₂—, —C(R^{12c})(R^{12d})— or C₂₋₄ alkylene optionally substituted by one or more substituents that are halo or —OC₁₋₃ alkyl;
(vi) L² is a 4-, 5- or 6-membered aromatic or non-aromatic cyclic linker group, optionally containing one or two heteroatoms and optionally substituted by one or more substituents that are halo or C₁₋₃ alkyl optionally substituted by one or more fluoro atoms;
(vii) L² is a 4-, 5- or 6-membered aromatic or non-aromatic cyclic linker group, optionally containing one or two heteroatoms that are nitrogen, oxygen or sulfur and optionally substituted by one or more substituents that are halo or C₁₋₃ alkyl optionally substituted by one or more fluoro atoms;
(viii) R^{5a} and R^{5b} independently are a linear, branched or cyclic alkyl;
(ix) R¹ is one, two or three substituents;
(x) R¹ is Cl or F;
(xi) R² is C₃₋₄ cycloalkyl;
(xii) R^{5a} and R^{5b} independently are linear, branched or cyclic alkyl;
(xiii) R^{5a} and R^{5b} independently are optionally substituted by one or more F;
(xiv) R⁹ is —C₁₋₄ alkyl substituted by one or more F;
(xv) R^{10a} and R^{10b} independently are C₁₋₄ alkyl substituted by one substituent;
(xvi) R^{10a} and R^{10b} independently are —O—C₁₋₄ alkyl substituted by one substituent; and/or
(xvii) Het¹, Het² and Het³ independently are a 5- or 6-membered aromatic ring containing one or two heteroatoms that are nitrogen, oxygen or sulfur.

18. The compound according to claim 2, wherein:
(i) L¹ is —C(R^{12a})(R^{12b})— or C₂₋₄ alkylene optionally substituted by one or more substituents that are halo or —OC₁₋₃ alkyl;
(ii) L² is a direct bond, —O—, —OCH₂—, —C(R^{12c})(R^{12d})— or C₂₋₄ alkylene optionally substituted by one or more substituents that are halo or —OC₁₋₃ alkyl;
(iii) L² is a 4-, 5- or 6-membered aromatic or non-aromatic cyclic linker group, optionally containing one or two heteroatoms and optionally substituted by one or more substituents that are halo or C₁₋₃ alkyl that is itself optionally substituted by one or more fluoro atoms;
(iv) L² is a 4-, 5- or 6-membered aromatic or non-aromatic cyclic linker group, containing one or two heteroatoms that are nitrogen, oxygen or sulfur;
(v) R¹ is one, two or three substituents;
(vi) R¹ is Cl or F;
(vii) R^{5a} and R^{5b} independently are —C₁₋₄ alkyl substituted by one or more F;
(viii) R⁹ is —C₁₋₄ alkyl substituted by one or more F;
(ix) R^{10a} and R^{10b} independently are C₁₋₄ alkyl substituted by one substituent;
(x) R^{10a} and R^{10b} independently are —O—C₁₋₄ alkyl substituted by one substituent;
and/or
(xi) Het¹, Het² and Het³ independently are a 5- or 6-membered aromatic ring containing one or two heteroatoms that are nitrogen, oxygen or sulfur.

19. The method according to claim 13, wherein the anti-mycobacterial agent is an anti-tuberculosis agent.

20. The combination according to claim 14, wherein the anti-mycobacterial agent is an anti-tuberculosis agent.

21. The product according to claim 15, wherein the anti-mycobacterial agent is an anti-tuberculosis agent.
